(19) United States
(12) Patent Application Publication
(10) Pub. No.: US 2019/0358175 A1
(43) Pub. Date: Nov. 28, 2019

(54) COMPOSITIONS AND METHODS OF TREATING CHRONIC PAIN BY ADMINISTERING PROPOFOL DERIVATIVES

(71) Applicants: THE TRUSTEES OF COLUMBIA UNIVERSITY IN THE CITY OF NEW YORK, New York, NY (US); CORNELL UNIVERSITY, New York, NY (US)

(72) Inventors: Gareth R. Tibbs, New York, NY (US); Pamela Flood, Closter, NJ (US); Peter Goldstein, Scarsdale, NY (US)

(21) Appl. No.: 16/457,015

(22) Filed: Jun. 28, 2019

Related U.S. Application Data
(62) Division of application No. 13/389,507, filed on Sep. 21, 2012.
(60) Provisional application No. 61/273,912, filed on Aug. 11, 2009.

(51) Int. Cl. A61K 31/05

(52) U.S. Cl. CPC A61K 31/05

(57) ABSTRACT

Compositions and methods for managing or treating chronic pain are provided. More particularly, methods are provided for managing or treating chronic pain by administering to a patient in need thereof an effective amount of propofol or a propofol derivative having limited anesthetic properties. Methods of modulating HCN channel gating are also provided. Pharmaceutically acceptable compositions for, e.g., modulating HCN channel gating are further provided.

### CROSS-REFERENCE TO RELATED APPLICATIONS

The present application is a divisional of U.S. application Ser. No. 13/389,507, filed Sep. 21, 2012, which is a National Stage of International Application No. PCT/US2010/045064, filed Aug. 10, 2010, which claims priority to U.S. Provisional Patent Application No. 61/273,912, filed Aug. 11, 2009. The entire contents of the above applications are hereby incorporated by reference as if recited in full herein.

### FIELD OF THE INVENTION

The present invention relates to compositions and methods for managing or treating chronic pain and associated symptoms. More particularly, the present invention relates to a composition for and a method of managing or treating chronic pain including administering to a patient in need thereof an effective amount of propofol or a propofol derivative. Methods of treating or ameliorating chronic pain or modulating HCN channel gating are also provided. Pharmaceutically acceptable compositions for modulating HCN channel gating are also provided.

### BACKGROUND OF THE INVENTION

Pain may be divided into two general categories. Acute pain is characterized by rapid onset, high intensity, and generally short duration. Acute pain, for example, may be associated with trauma or surgery. Chronic pain, on the other hand, is persistent in that it lasts longer than the normal course of pain for a particular injury and may have a mild to high intensity. Chronic pain is associated with many conditions, including inflammation, e.g., back pain or arthritis; neuralgia, e.g., post-surgery or post-injury; complex regional pain syndromes, e.g., causalgia, reflex sympathetic dystrophy; cancer pain; phantom pain, e.g., post-amputation pain; neuropathy, e.g., diabetic or ischemic; or spinal cord injury.

Chronic pain also carries symptoms that may be even further debilitating. Chronic pain sufferers may exhibit enhanced sensitivity to painful stimulus (hyperalgesia); painful sensation to normally non-painful stimulus (allodynia); burning sensation; and unusual nociceptive descriptors (stabbing, sharp, throbbing, etc.). In addition, chronic pain may also have additional physiological consequences, for example, a trigger point producing pain (myofascial pain or radicular pain) or sympathetic dystrophy (warm/cold extremities, joint stiffness, or bone demineralization).

Not surprisingly, the two types of pain are treated or managed differently. Acute pain responds well to medication. Moreover, the fact that acute pain is often associated with injury or surgery means that acute pain is often treated in the clinical setting. Accordingly, acute pain may be treated with opioids, such as codeine or morphine or with anesthesia, for example, with surgery. These treatments, however, have significant side effects. Opioids may suppress respiration and cardiac functions, alter consciousness, and may interfere with both gastrointestinal and urinary function. General anesthesia results in loss of consciousness, may cause suppression of respiration and cardiac function, and requires close monitoring during application.

In the treatment of acute pain, such side effects are accepted because the relief of the patient's pain is paramount and the required equipment and personnel are present to closely track the patient's status. With chronic pain, these side effects may not be acceptable.

Treatment of chronic pain focuses on treating the patient so that (s)he may resume a normal daily routine, as much as, possible. The close monitoring found with the management of acute pain is likely to be incompatible with a normal daily routine and anesthesia would prevent anything resembling a normal daily routine. The altered consciousness produced by some of these treatments, e.g., confusion and sleepiness, may make many common tasks dangerous or impossible. For example, operating machinery or driving an automobile would be ill advised after ingestion of an opioid. Moreover, many of the treatments for acute pain may be addictive and are, therefore, not appropriate for long term use.

The various classes of analgesics have different benefits and side effects as shown in Table 1.

TABLE 1
Analgesic classes - benefits and side effects.

| Analgesic | Benefits | Side Effects |
|---|---|---|
| Non-Opioids | Can control mild to | Can cause slow blood |
| acetaminophen, | moderate pain; some | clotting and upset |
| aspirin, ibuprofen, | versions can be bought | stomach, bleeding in |
| non-steroidal anti- | without prescription | the stomach, and |
| inflammatory drugs | | kidney problems |
| Opioids | Can control moderate | Can cause constipa- |
| morphine, | to sever pain without | tion, sleepiness, |
| hydromorphone, | bleeding | nausea and vomiting, |
| oxycodone, codeine, | | itchiness and urinary |
| fentanyl, methadone | | problems; may also slow |
| | | breathing when first |
| | | taken |
| Antidepressants | Can help control tingling | Can cause dry mouth, |
| amitriptyline, | or burning pain from | sleepiness, constipa- |
| imipramine | nerve injury; may | tion, and dizziness on |
| | improve sleep | standing up |
| Anticonvulsants | Can help control tingling | Can effect liver and |
| carbamazepine, | or burning pain from | blood cell function |
| phenytoin | nerve injury | |
| Steroids | Can help relieve bone | May cause confusion, |
| prednisone, | pain and pain caused | fluid buildup, and |
| dexamethasone | by spinal cord and | bleeding and irrita- |
| | brain tumors | tion in stomach |

Analgesics are selected and combined to manage chronic pain while minimizing side effects. For example, the World Heath Organization has designed an “Analgesic Ladder” for treatment of the pain associated with cancer, as shown in FIG. 6. Examples of adjuvants in this ladder include the antidepressants, anticonvulsants, and steroids shown in Table 1.

The current understanding of the production of a painful sensation is a modulatable pathway model (FIG. 7). That is, pain is not the simple reporting of a stimulus to the brain that is perceived as pain. Rather, production of pain follows a complex pathway that allows for attenuation or modulation of the signal before it is delivered to the brain. As shown in FIG. 7, a painful stimulus stimulates a nociceptor to produce a pain signal along a nerve bundle to nerve cells in the dorsal horn of the spinal cord. In addition, inhibitory fibers from the brain may produce signals that inhibit the propagation of the pain signal along the nerve bundle and/or suppress the production of a signal from the dorsal horn to the brain. Likewise, a sensory receptor is stimulated to produce a signal passed from the periphery to a local inhibitory interneuron. The local inhibitory interneuron may then produce signals that inhibit the propagation of the pain signal along the nerve bundle fiber and/or suppress the production of a signal from the dorsal horn to the brain. In this manner, the pain signal is modulated before it is passed along the ventral nerve to the brain.

In normal pain pathways (FIG. 8), a high intensity stimulus produces a signal perceived as pain through high threshold A-delta nociceptor (“first pain”) and C nociceptor (“second pain”) nerve fibers. A low intensity stimulus, on the other hand, produces a signal through a low threshold A beta fiber to produce an innocuous sensation.

Chronic pain is produced by one of two aberrant pathways as shown in FIG. 9. Chronic pain may be produced from a low intensity stimulus either through peripheral sensitization or through central sensitization. In peripheral sensitization, the high threshold A-delta and C nociceptor nerve fibers, which normally produce signals only in response to a high intensity stimulus, are sensitized. The sensitized high threshold A-delta and C nociceptor nerve fibers produce signals in response to a low intensity stimulus that is passed to the dorsal horn neuron and thereby to the brain as if it was a response to a high intensity stimulus. In central sensitization, the dorsal horn neuron is hyperexcitable and a normal signal from low threshold A beta nerve fibers in response to a low intensity stimulus is reported to the brain as a signal from a high intensity stimulus by the dorsal horn neuron.

Key events in the development and maintenance of neuropathic pain are the onset of peripheral and central sensitization—enhanced excitability or sensitivity of injured or adjacent peripheral nerves and increased spinal or supraspinal processing of painful information, respectively (Costigan et al., 2009; Markman et al., 2006; Dray, 2008; Scholz et al., 2002; Campbell et al., 2005; Beggs et al., 2006; Davis et al., 2005; Heinricher et al, 2009; Ossipov et al., 2000; Vanegas et al., 2004; Scholz et al., 2007; Romanelli et al., 2004; Willis et al., 2007). An upregulation of the hyperpolarization-activated current (I_{H}) pacemaker current appears to contribute to peripheral sensitization by promoting the emergence of aberrant hyperexcitability of peripheral C, Aδ (and Aβ (Maher et al., 2009)) nociceptors and normally non-nociceptive A-type (e.g. Aβ mechanoreceptor) neurons (Swartz, 2008; Markman et al., 2006; Scholz et al., 2002; Dworkin et al., 2003; Maher et al., 2009; Djouhri et al., 2004; Liu et al., 2002; Djourhir et al., 2006; Liu et al., 2000A; Liu et al., 2000B; Roza et al., 2003; Xie et al., 2005; Song et al., 1999; Chaplan et al., 2003; Jiang et al., 2008; Kajander et al., 1992; Wu et al., 2001; Sun et al., 2005; Kitagawa et al., 2006).

Regardless of central or peripheral sensitization, the cell bodies of the peripheral sensory nerves or the dorsal root ganglion (DRG) play an important role in the pathogenesis of chronic pain. Molecular and cellular changes in the neurons of the DRG that enhance excitability are likely to result in pain, while decreasing DRG excitability may inhibit pain.

Hyperpolarization-activated cyclic nucleotide-modulated (HCN) channels have been implicated in the occurrence of chronic pain. When activated, HCN channels produce an I_{H} current in the cell. Yao et al. report that chronic compression of DRG in the L₄-L₅ region of the spine produces hyperalgesia in rats. Chronic compression of the DRG also increases the density of the I_{H} current in the DRG because of an increase in I_{H} conductance and an increase in the rate of activation of I_{H} current. Accordingly, it appears that chronic compression of the DRG upregulates HCN channels and produces hyperalgesia by enhanced excitability of the DRG (Yao et. al., 2003).

HCN channels are also implicated in neuropathic pain after nerve injury. Nerve injury increases pacemaker currents in DRG, spontaneous firing of the damaged nerve, and results in hypersensitivity to light touch. Blockage of the HCN channels, especially the HCN1 channel, reverses this hypersensitivity and decreases the ectopic firing frequency. (Chaplan et al., 2003).

In animal models of neuropathic pain, hyperexcitability and ectopic activity in primary afferents contribute to peripheral sensitization (Costigan et al., 2009; Scholz et al., 2002; Campbell et al., 2006; Beggs et al., 2006; Dray et al., 2008; Dib-Hajj et al., 2009). Convergent lines of evidence suggest that such changes are driven, at least in part, by changes in HCN channel expression and function. First, in situ hybridization (Kouranova et al., 2009), immunohistochemical (Kouranova et al., 2009; Tu et al., 2004; Jiang et al., 2008), and electrophysiological data (Kouranova et al., 2009; Mayer et al., 1983; Pearce et al., 1994; Scorggs et al., 1994; Momin et al., 2008) show sensory neurons express HCN channels and that injury results in altered HCN subunit trafficking (Jiang et al., 2008; Chaplan et al., 2003; Wells et al., 2007) and an enhancement of the I_{H} current amplitude (Chaplan et al., 2003; Yao et al., 2003; Kitagawa et al., 2006; Tsuboi et al., 2004). Second, sensory cell hyperexcitability is inhibited by superfusion with ZD7288 (Jiang et al., 2008; Chaplan et al., 2003; Yao et al., 2003; Lee et al., 2005; Sun et al, 2005), a selective but pan-isoform inhibitor of HCN channels (Shin et al., 2001; Cheng et al., 2007; Stieber et al., 2005), while systemic but not central ZD7288 alleviates mechanical allodynia (Chaplan et al., 2003; Lee et al., 2005; Luo et al., 2007). Last, deletion of the HCN1 gene partially blocks development of cold allodynia (Orio et al., 2009).

Blockage of HCN channels reduces allodynia produced, e.g., by damage to the sciatic nerve or incision of the hind paw in rats. Administration of a selective inhibitor of the I_{H} current (e.g., by blockage of the peripheral HCN channels) reduces mechanical allodynia as measured by the force required to produce withdrawal of the limb after injury. (Dalle et al., 2005).

Four HCN subunits, each of which has six transmembrane helices (S1-S6) and cytoplasmic amino and carboxy termini, form an I_{H} channel (see FIGS. 10A and 10B). The pore of the channels is formed from a four-fold assembly of the S5-S6 domain (black elements in FIG. 10B) while the voltage-sensing apparatus (formed from the first four transmembrane helices of each subunit) is loosely attached to the pore module (white elements in FIG. 10B). Gating of HCN channels involves coupling of the independent motions of the four voltage sensing “paddles” (Bell et al., 2003; Vemana et al., 2002) to a concerted opening of a helical bundle formed from the apposition of the cytoplasmic ends of the S6 helices (Shin et al., 2001; Rothberg et al., 2002; Yellen, 2002; Yellen, 1998; MacKinnon, 2003).

Modulation of HCN channel gating by cAMP is mediated via nucleotide binding to a gating ring that lies distal to the pore lining S6 helix (Santoro et al., 1999; Robinson et al., 2003; Craven et al., 2005), while the effects of intracellular protons have been ascribed to a histidine residue lying at the intracellular end of the S4 helix. cAMP binding and proton binding can be selectively eliminated by mutation of an arginine in the cyclic nucleotide-binding domain (CNBD) and of the histidine at the end of S4, respectively. While the mechanisms of coupling of acidic lipids such as 4,5-PIP2 and arachidonic acid have not been well defined, a primary seat of action of these lipophilic modifiers lies within the conserved core domain of the channels. This observation is consistent with the presence of lipids intercalating within and between the voltage sensors and pore domains of the Kv channel crystal structure (FIGS. 11A and 11B) and the sensitivity of HCN gating to acyl chain chemistry (unpublished observations).

The greatest variation between the four HCN isoforms lies in the distal half of the N terminus and the sequence lying distal to the CNBD in the C-terminus. While these elements appear to be important for coupling to auxiliary proteins, the basic properties of channels formed from the different subunits are determined by small differences in the conserved core (proximal to the Nv and Cv boundaries in FIG. 10A). Upon heterologous expression, all four HCN isoforms have been shown to form functional homomeric channels. Moreover, with the exception of the HCN2/HCN3 combination, the subunits promiscuously coassemble (Much et al., 200; Altomare et al., 2003). Coassembled channels incorporating the HCN1 subunit retain propofol sensitivity (Chen et al., 2005). These observations have important implications with respect to the method of targeting native I_{H} channels.

Based on in situ hybridization, qPCR, immunohistochemistry and electrophysiology, the following HCN subunit expression patterns are found. In large and medium (Aβ, Aδ) cutaneous exteroceptive primary sensory neurons, HCN1 and 2 dominate while HCN3 levels are low. In smaller cells (Aδ, C) the pattern appears reversed (Chaplan et al., 2003; Jiang et al., 2008; Wells et al., 2007; Mom in et al., 2008; Moosmang et al., 2001; Tu et al., 2004; Kouranova et al., 2008; Obreja et al., 2008; Kang et al., 2004), albeit HCN1 may identify noxious cold C cells (Orio et al., 2009; Momin et al., 2008). HCN4 expression is minimal in these cells (Chaplan et al., 2003; Moosmang et al., 2001; Tu et al., 2004; Kouranova et al., 2008). HCN2 and 4 predominate in interoceptive aspects of somesthesis (Doan et al., 2004). In human and mouse heart, HCN2 and 4 predominate with little or no HCN1 or 3 (Wickenden et al., 2009; Moosmang et al., 2001; Mistrik et al., 2005). Moreover, HCN1-4 are all variably expressed in the CNS (Santoro et al., 1997; Santoro et al., 1998; Ludwig et al., 1998; Moosmang et al., 2001; Mistrik et al., 2005; Santoro et al., 2000; Monteggia et al., 2000; Mossmang et al., 1999; Ishii et al., 1999; Ludwig et al., 1999; Seifert et al., 19999; Ludwig et al., 2003; Stieber et al., 2003, Abbas et al., 2006; Ying et al., 2007). Furthermore, I_{H} recorded at a cell's soma tends to behave as anticipated from the HCN subunit profile. However, in extended cells the somatic current can be small or absent due to channel localization at electrically distant sites e.g., pre and post synaptic membranes (Biel et al., 2009; Magee, 1999; Berger et al., 2001; Poolos et al., 2002; Lorincz et al., 2002; Ulrich, 2002; Berger et al., 2003; Surges et al., 2004; Migliore et al., 2004; Abbas et al., 2006; Kole et al., 2006; Tsay et al., 2007; Johnston et al., 2008; Southan et al., 19998; Beaumont et al., 2000; Southan et al., 2000; Bender et al., 2001; Cuttle et al., 2002; Migliore et al., 2002; Muller et al., 2003; Klar et al., 2003; Lujan et al., 2005, Bender et al., 2007; Lipscombe et al., 2009).

There is a dearth of isoform selective antagonists for the I_{H} channels. A number of HCN antagonists have been developed (DiFrancesco, 2006; Barbuti et al., 2007; Bucchi et al., 2007; Bucchi et al., 2002; Bucchi et al., 2006). However, these pore blockers (e.g. ZD7288) have no isoform selectivity (Shin et al., 2001; Cheng et al., 2007; Stieber et al., 2005; Bucchi et al., 2006) and are (in keeping with their original purpose) strongly bradycardic. As such they are compromised with respect to development as analgesics for neuropathic pain.

In view of the foregoing, it would be advantageous to provide a method for managing or treating chronic pain and other disorders that does not suffer from the side effects of traditional analgesics, such as for example, those described above.

### SUMMARY OF THE INVENTION

Ideally, a neuropathic pain analgesic that targets I_{H} should preferentially inhibit channels comprised of, or containing, the HCN1 isoform (thereby sparing the homologous cardiac current) and be restricted to the periphery (thereby sparing I_{H} in central neurons, including those that rely on the HCN1 isoform). To date only the intravenous anesthetic propofol has shown evidence of HCN isoform-selective antagonism, displaying marked preference for HCN1 over HCN2, 3 or 4 (Cacheaux et al., 2005; Chen et al., 2005), but see Ying et al. (2006). However, the general hypnotic properties of propofol resulting from its ability to allosterically enhance GABA_{A}-R activity (Vanlersberghe et al., 2008; Franks et al., 2008; Franks, 2006; Hemmings et al., 2005; Rudolph et al., 2004; Trapani et al., 2000; Jurd et al., 2003), clearly limit its utility as an analgesic. Importantly, GABA_{A}-Rs show a pronounced sensitivity to the nature of the alkyl substitutions present on the phenol ring consistent with the compounds binding to a sterically-defined pocket. Thus, while both 2,6- and 2,4-di-butylphenols are potent allosteric activators of the chloride channels and are potent anesthetics when the alkyl groups are in the secondary configuration (2,6-di-sec-butylphenol (2,6-DSBP) and 2,4-di-sec-butylphenol (2,4-DSBP)), the di-tert versions of these compounds (2,6-di-tert-butylphenol (2,6-DTBP) and 2,4-di-tert-butylphenol (2,4-DTBP)) are neither anesthetics nor efficacious allosteric modifiers of GABA_{A}-Rs (James et al, 1980; Krasowski et al., 2001). These findings suggest modest modifications could yield novel, non-anesthetic, therapeutic agents that inherit the safety profile of the widely used parental molecule, propofol.

In view of the foregoing, one embodiment of the present invention is a method of managing or treating chronic pain comprising administering to a patient in need thereof an effective amount of propofol or a propofol derivative having limited general anesthetic properties.

Another embodiment of the invention is a method of modulating HCN channel gating. This method comprises providing to an HCN channel an effective amount of propofol or a propofol derivative having limited general anesthetic properties.

A further embodiment of the invention is a method of inhibiting an HCN1 channel without enhancing a gamma-aminobutyric acid-A (GABA-A) receptor. This method comprises providing to an HCN channel an effective amount of a propofol derivative having limited general anesthetic properties.

### BRIEF DESCRIPTION OF THE DRAWINGS

FIG. 1 shows the structures of various propofol derivatives according to the present invention.

FIGS. 2A-2D show that propofol preferentially inhibits HCN1 with little efficacy against HCN2, 3 or 4 channels. FIG. 2A shows two-electrode voltage clamp (TEVC) current records (LEFT) and an expanded view of the tail currents (RIGHT) before (TOP) and after (BOTTOM) 20-minute incubation in 20 μM propofol for full length HCN1. In each case, the red trace highlights the currents recorded upon activation of the channels at −75 mV. FIG. 2B shows the steady-state activation curves for the cells shown in FIG. 2A. Fits of the Boltzmann function are superimposed and show that propofol results in a marked hyperpolarization of activation gating of HCN1. FIG. 2C shows dose-response data for propofol modulation of V_{1/2} in HCN1. Fit of the Hill function to the HCN1 data is superimposed. FIG. 2D shows the shift in the midpoint of activation of HCN1, 2, 3 and 4 following incubation in DMSO vehicle or 20 μM propofol. Asterisk (*) shows statistical significance versus zero drug which is, in turn, vehicle with respect to no addition control. Data are means±SEM of recordings from 4 to 9 separate cells. The hyperpolarization of the V_{1/2} was significantly different from the DMSO control population at concentrations of 3 μM and above (P<0.001). The concentration of propofol required for half maximal effect was about 13 μM.

FIGS. 3A-3D show that 2,4-di-tert-butylphenol and 2,6-di-tert-butylphenol preferentially inhibit HCN1 with little efficacy against HCN2, 3 or 4 channels and do so with a higher potency than does parental propofol. TEVC current records (LEFT) and an expanded view of the tail currents (RIGHT) before (TOP) and after (BOTTOM) 20 minute incubation in either DMSO (Top in gray) or 10 μM 2,6-di-tert-butylphenol for full length HCN1. In each case, the red trace highlights the currents recorded upon activation of the channels at −65 mV. Recordings were obtained from two separate cells, but the cells were from the same donor frog and were acquired on the same day. FIG. 3B shows steady-state activation curves for the cells shown in FIG. 3A. Fits of the Boltzmann function (superimposed lines) yielded values of the V_{1/2} and slope of −60.6 and 8.7 mV (mean −64.8 mV±1.9 and 7.5 mV±0.3; n=14) versus −90.8 and 9.7 mV (mean −88.3 mV±1.4 and 9.6 mV±0.1; n=6) in the presence of DMSO (open circles) and 10 μM 2,6-DTBP (filled circles), respectively. Therefore, 2,6-DTBP results in a marked hyperpolarization of activation gating of HCN1. FIG. 3C shows dose-response data for 2,6-di-tert-butylphenol (2,6 di-tert), 2,6-di-sec-butylphenol (2,6 di-sec), 2,4-di-tert-butylphenol (2,4 di-tert), 2,4-di-sec-butylphenol (2, 4 di-sec) modulation of V_{1/2} in HCN1. The solid line represents a fit of the Hill function to the 2,6-di-tert-butylphenol with a determined EC₅₀ of about 2.3 μM. The dashed lines are the 2,6-DTBP fit line offset by 2-, 15-, and 23-fold for 2,4-DTBP, 2,6-DSBP, and 2,4-DSBP, respectively. Because the ΔV_{1/2} elicited by 20 μM 2,6-DTBP was similar irrespective of whether the compound was solubilized in DMSO or DHβCD, these values were pooled here. For DTBPs, the shift in V_{1/2} was significant at 1 μM and higher, but for DTBPs, significance was only observed at 20 μM. FIG. 3D shows sensitivity of gating of HCN1, 2, 3 and 4 to 2,6-di-tert-butylphenol with respect to DMSO vehicle. Asterisk (*) shows statistical significance versus zero drug which is, in turn, vehicle with respect to no addition control. These findings show that 2,6-di-tert-butylphenol has the strongest potency and highest efficacy of the four derivatives with respect to modification of HCN1 gating. These findings also show that 2,6-di-tert-butylphenol selectively antagonizes gating of the HCN1 channel.

FIGS. 4A-4E show subanesthetic concentrations of propofol relieves thermal hyperalgesia and mechanical allodynia in the nerve ligation neuropathic pain models without markedly altering normal nociceptive responses. The plot on the top left (FIG. 4A) shows 7-days post nerve ligation, the ipsilateral hind paw in response to a stimulus of 15% or 30% of the maximum of the heat source. The ipsilateral (damaged) paw shows a far faster withdrawal at the lower light intensity (representing a neuropathic response) while the latency of the ipsilateral and contralateral paws are similar at higher (nociceptive) stimulus intensities. The plot in the top middle (FIG. 4B) shows the population mean responses of the ipsilateral and contralateral paws in response to a 15% stimulus following no treatment (baseline) or Intraperitoneal (i.p.) injection of saline or the indicated doses of propofol. These subanesthetic doses of propofol relieve the neuropathic behavior (slowing ipsilateral withdrawal) without significantly altering nociceptive behavior (no slowing of the contralateral withdrawal). The plot on the top right (FIG. 4C) shows the ipsilateral/contralateral hind paw withdrawal latency (HPWL) ratio determined from the same data presented in the above panel. This plot shows the within animal significance of the selective slowing of the ipsilateral response. The plot on the bottom left (FIG. 4D) shows the mean number of withdrawals of ipsilateral (red) and contralateral (blue) paws in 7-day post nerve ligation animals in response to irritation of the paw with fibres of the indicated strength. Within each group, the relief of the nocifensive behavior following i.p. injection of a cumulative dose of propofol is shown by increasing color density in each histogram cluster. The plot on the bottom right (FIG. 4E) shows the number of withdrawals observed in the absence versus presence of drug determined within each animal and then averaged across the higher fiber strengths as indicated.

FIGS. 5A-5B show that i.p. injection of the non-anesthetic, GABA-disabled propofol derivative, 2,6-di-tert-butylphenol relieves thermal hyperalgesia in nerve ligation neuropathic mice without altering normal nociceptive behavior. The plot on the left (FIG. 5A) shows the population mean responses of the ipsilateral and contralateral paws in response to a 15% stimulus following no treatment (baseline) or i.p. injection of the indicated doses of 2,6-di-tert-butylphenol. These non-toxic doses of compound significantly relieve the neuropathic behavior (slowing ipsilateral withdrawal) without significantly altering nociceptive behavior (no slowing of the contralateral withdrawal). The plot on the right (FIG. 5B) shows the ipsilateral/contralateral HPWL ratio determined from the same data presented in the left panel. This plot shows the within animal significance of the selective slowing of the ipsilateral response.

FIG. 6 shows the World Health Organization analgesic ladder for the treatment of the pain associated with cancer.

FIG. 7 shows a schematic representation of the pathway for sensory transmission from the periphery to the CNS.

FIG. 8 shows a schematic representation of normal sensory transmission from the periphery to the CNS.

FIG. 9 shows a schematic representation of potential sites of sensitization within sensory transmission pathways leading to development of neuropathic pain.

FIGS. 10A-10B show the architecture of an HCN channel. FIG. 10A shows the HCN subunit, and FIG. 10B shows the tetrameric HCN channel. The voltage sensing S1-S4 motifs (white boxes and circles) form a module that surrounds the pore forming S5-S6 domain (black boxes and circles). These membrane embedded elements alone are required for HCN1 to sense propofol.

FIG. 11A shows the structure of the Kv1.2-2.1 chimera showing the presence of intercalated lipids (teal), pore domain (yellow), and voltage sensor (buff and purple) (Swartz, 2008). FIG. 11B shows a different view of a portion of the Kv1.2-2.1 chimera.

FIGS. 12A-12D show that subhypnotic doses of propofol selectively suppresses partial sciatic nerve ligation (PNL)-induced mechanical allodynia and hyperalgesia with respect to mechanical nociception. FIG. 12A shows the probability of withdrawal of the ipsilateral paw (P_{W IPSI}) and the probability of withdrawal of the contralateral paw (P_{W CONTRA}) as a function of stimulus fiber strength determined before and after i.p. administration of propofol. FIG. 12B shows P_{W IPSI} and P_{W CONTRA} as a function of control P_{W CONTRA}. FIG. 12 C shows P_{W IPSI} and P_{W CONTRA} as a function of PW observed in the cognate paw before propofol. FIG. 12D shows log it transformation of P_{W IPSI} and P_{W CONTRA} in the absence or presence of propofol. The color gradations have the same meaning with respect to dose as in FIG. 12A. Here the x-axis represents a PW of 0. Asterisks and crosses indicate PW values statistically different from control P_{W IPSI} and P_{W CONTRA}, respectively. In FIG. 12A, tests are performed separately for each stimulus intensity; in FIG. 12B, tests are with respect to P_{W CONTRA}; in FIG. 12C, tests are within paw only. Because data in FIG. 12D are simply a transformation of those in FIG. 12A, statistical indicators are omitted for clarity. Data are from 15 mice (except for 0.6 g stimulus, which was for 11 mice).

FIGS. 13A-13D show that subhypnotic doses of propofol selectively ameliorate PNL-induced thermal hyperalgesia with respect to thermal nociception. FIGS. 13A and 13B show hind paw withdrawal latency (HPWL) as a function of heat source intensity determined before and after i.p. propofol. FIG. 13C shows HPWL of the ipsilateral paw (HPWL_{IPSI}) and HPWL of the contralateral paw (HPWL_{CONTRA}) as a function of control HPWL_{CONTRA}. FIG. 13D shows HPWL_{ISPSI} and HPWL_{CONTRA} as a function of HPWL observed in the cognate paw before propofol. Asterisks and crosses indicate HPWL values statistically different from control HPWL_{IPSI} and control HPWL_{CONTRA}, respectively. In FIG. 13C, tests are with respect to PW CONTRA; in FIG. 13D, tests are within paw only. Data are from 10 (30%) or 14 (15%) mice.

FIGS. 14A-14D show that 2,6-DTBP selectively suppresses PNL-induced mechanical allodynia and hyperalgesia with respect to mechanical nociception. FIG. 14A shows P_{W IPSI} and P_{W CONTRA} as a function of stimulus fiber strength determined before and after i.p. administration of 2,6-DTBP. FIG. 14B shows P_{W IPSI} and P_{W CONTRA} as a function of control P_{W CONTRA}. FIG. 14C shows P_{W IPSI} and P_{W CONTRA} as a function of PW observed in the cognate paw before administration of 2,6-DTBP. FIG. 14D shows log it transformation of P_{W IPSI} and P_{W CONTRA} in the absence or presence of 2,6-DTBP. The color gradations have the same meaning with respect to dose as in FIG. 14A. Here the x-axis represents a PW of 0.1. Asterisks and crosses have same meaning as in FIGS. 12A-12D. Data are from 10 mice.

FIGS. 15A-15D show that 2,6-DTBP selectively ameliorates PNL-induced thermal hyperalgesia with respect to thermal nociception. FIGS. 15A and 15B show HPWL as a function of heat source intensity determined before and after i.p. 2,6-DTBP. FIG. 15C shows HPWL_{IPSI} and HPWL_{CONTRA} as a function of control HPWL_{CONTRA}. FIG. 15D shows HPWL_{IPSI} and HPWL_{CONTRA} as a function of HPWL observed in the cognate paw before i.p. administration of 2,6-DTBP. Asterisks and crosses have the same meaning as in FIGS. 13A-13D. Data are from 10 mice.

FIGS. 16A-16E show the stability and bioavailability of DHβCD solubilized 2,6-DTBP. FIGS. 16A-16C show representative gas chromatography (GC) chromatograms of 2,6-DTBP solubilized acutely in DMSO (FIG. 16A) or following solvation in DHβCD (FIG. 16B) with respect to a sample containing both DMSO and DHβCD but not 2,6-DTBP (FIG. 16C). The first large peak at 1 minute retention time (RT) is the solvent chloroform followed by a DMSO peak at about 1.5 minutes. The peak of the thymol internal standard (IS, 2.7 min RT, 100 nM) is in close proximity of the 2,6-DTBP peak (2.8 min RT). Note that, for clarity, the thymol internal standard was omitted from the sweep in panel FIG. 16C. FIGS. 16D and 16E show representative GC chromatograms of extracts taken from whole blood following injection with DHβCD vehicle (FIG. 16D) or 80 mg/kg (i.p.) 2,6-DTBP in DHβCD (FIG. 16E). The total 2,6-DTBP concentration in blood was determined to be 10.6 μM±4.9, mean±SEM, n=6. Such a concentration would be expected to yield a free concentration of 0.1 to 0.5 μM.

FIG. 17 shows a diagram depicting the origins of neuropathic pain and interdiction by non-anesthetic propofol derivatives.

FIG. 18 shows a diagram showing I_{H} channel physiology.

FIG. 19 shows the structure of certain propofol derivatives.

### DETAILED DESCRIPTION OF THE INVENTION

One embodiment of the present invention is a method of managing or treating chronic pain. This method comprises administering to a patient in need thereof an effective amount of propofol or a propofol derivative having limited general anesthetic properties.

The structure of propofol is shown below:

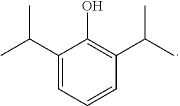

As used herein, “chronic pain” means pain that lasts longer than normal course of pain for a particular injury. Chronic pain intensity may vary from mild to high. Chronic pain includes neuropathic pain, which refers to a chronic pain of nerve origin.

As used herein, “patient” is a vertebrate, preferably a mammal, more preferably a human. Mammals include farm and sport animals, and pets.

As used herein, “limited” general anesthetic properties means at the amount administered to the patient, the propofol derivative has either no general anesthetic effect on a patient or a very limited effect, e.g., the patient does not lose consciousness, which requires no or minimal monitoring by a physician.

In one aspect of this embodiment, the propofol derivative comprises a compound of the formula (I):

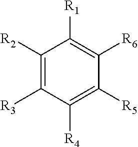

wherein
R₁ is selected from the group consisting of H and OH;
R₂, R₄, and R₆ are independently selected from the group consisting of H; —NR₁₀R₁₁, where R₁₀ and R₁₁ are independently selected from the group consisting of H and

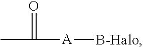

where A is a 5-membered heterocycle, B is a 6 membered aryl, and Halo is a halogen atom; C₁₋₄alkoxy; C₁₋₈alkyl, which is optionally substituted with one or more groups selected from the group consisting of —OH, —CF₃, carbonyl, —NH₂, and alkyne; C₁₋₄alkene optionally substituted with a 5- or 6-membered heterocycle, where from 0-2 carbon atoms of the heterocycle are optionally substituted with an atom selected from the group consisting of N, S, and O and one or more groups are pendant from a ring atom of the heterocycle, the pendant groups being independently selected from the group consisting of —H, —CH₃O, carbonyl, sulfonyl, —CH₃, —NH—OCH₃, and —CH₂CH₃; and —S—R₇—S—R₈ wherein R₇ is a C₁₋₄alkyl optionally substituted with C₁₋₄alkyl and R₈ is an aromatic ring optionally substituted with C₁₋₄alkyl or O—R₉, where R₉ is H or C₁₋₄alkyl optionally substituted with a carbonyl or —OH;
R₃ and R₅ are H;
or pharmaceutically acceptable salts thereof.

In another aspect of this embodiment, the propofol derivative is selected from the group consisting of:

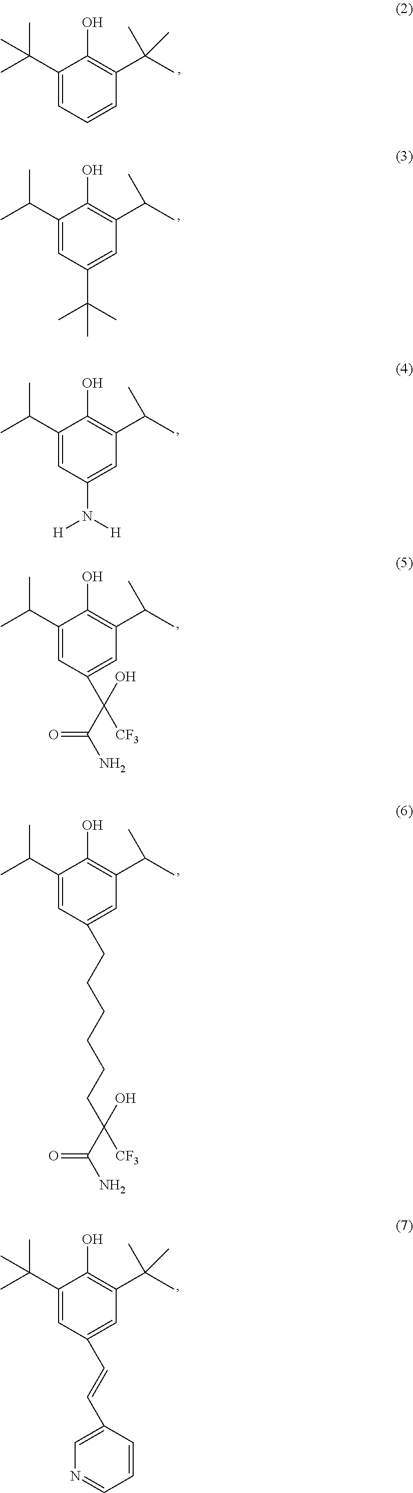

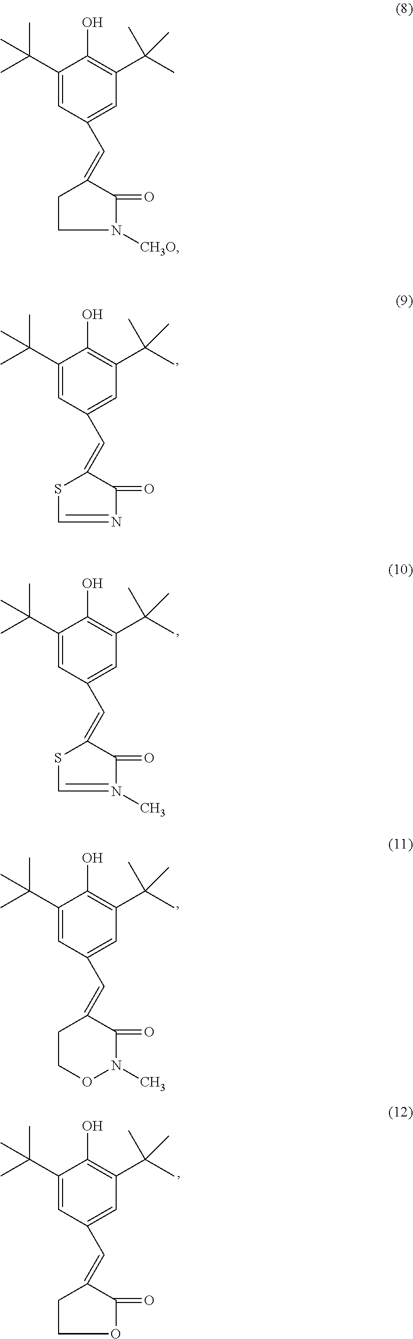

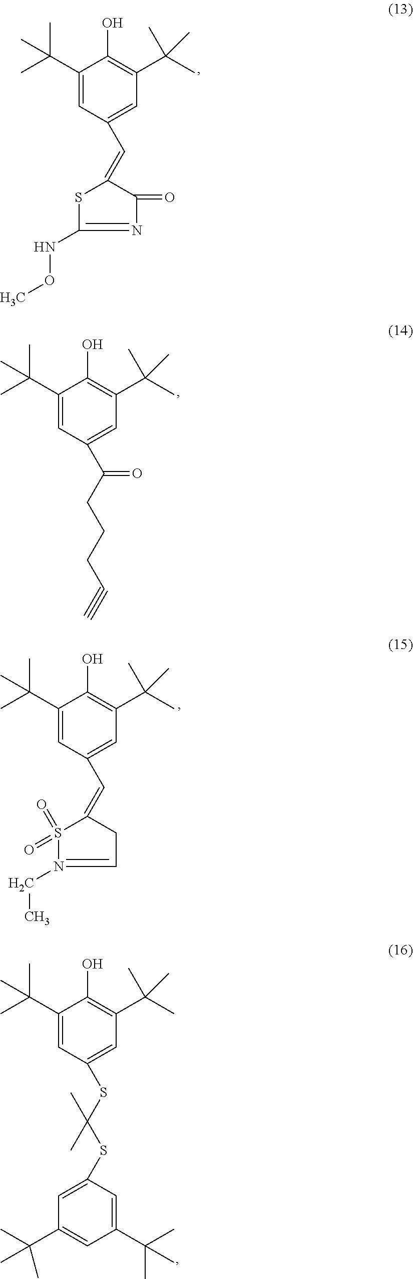

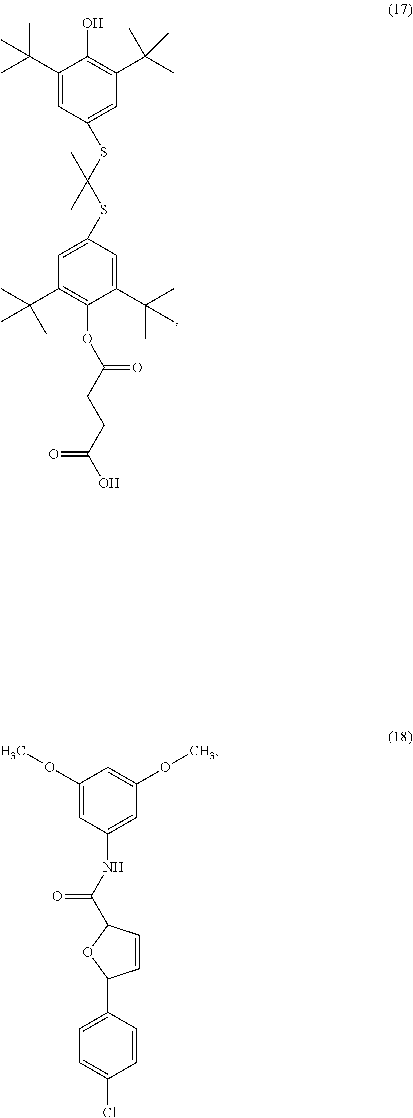

and pharmaceutically acceptable salts thereof.

In an additional aspect of this embodiment, the propofol derivative is selected from the group consisting of:

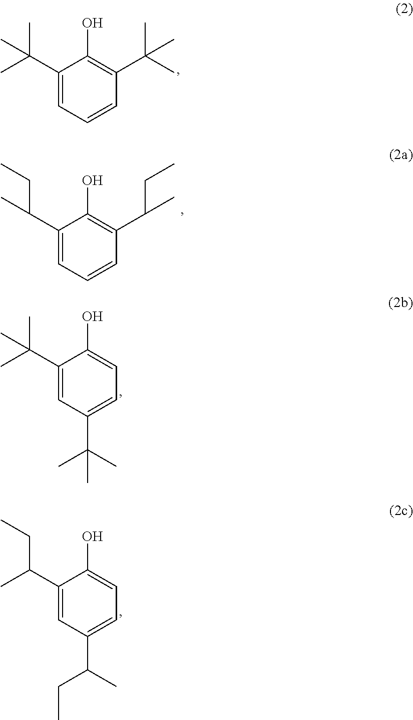

and pharmaceutically acceptable salts thereof.

In yet another aspect of this embodiment, the propofol derivative is

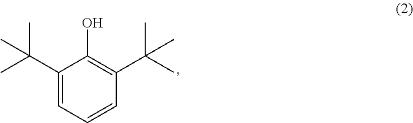

or a pharmaceutically acceptable salt thereof.

In a further aspect of this embodiment, the propofol derivative is selected from the group consisting of

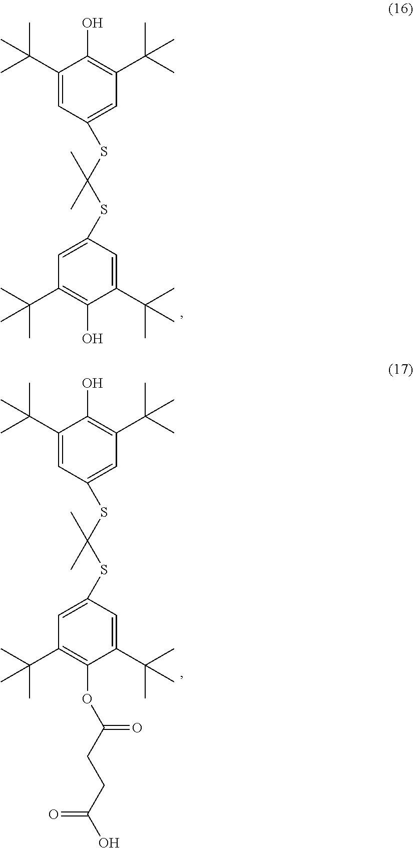

and pharmaceutically acceptable salts thereof.

In an additional aspect of this embodiment, the propofol derivative comprises a compound of the formula (II) or a compound of the formula (III):

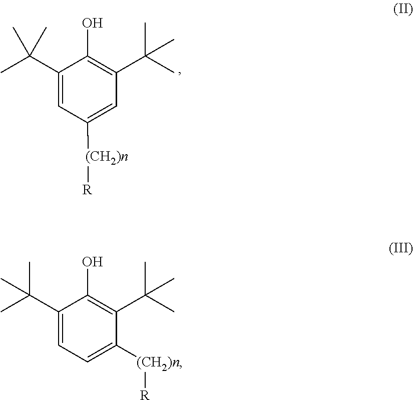

wherein n=2-36; and R is a positively charged group or atom;

or a pharmaceutically acceptable salt thereof. In one embodiment, n is from 2-18, such as 4, 8, or 12. In the present invention, the recitation of a range includes all numbers within the range, including the recited end points. Preferably, R is selected from the group consisting of NH₂, N(CH₃)₃, a guanidine group, an aromatic amino group, and a quaternary ammonium group.

In yet another aspect of this embodiment, the propofol or propofol derivative is administered as part of a pharmaceutically acceptable composition. Preferably, the pharmaceutically acceptable composition is administered in an unit dosage form. In the present invention, the propofol derivative is present in the unit dosage form at a total concentration of e.g., about 1 μM to about 20 μM, although other concentrations may be used at the physician's option, based on, e.g., the patient's weight, age, etc., such that a non-hypnotic dosage is administered. For example, propofol or a propofol derivative is administered at a dosage that is below a dosage that would induce general anaesthesia in a patient.

In an additional aspect of this embodiment, the chronic pain is a neuropathic pain characterized by one or more symptoms selected from the group consisting of persistent negative sensory perception, hyperalgesia, allodynia, burning sensation, and unusual nociceptive descriptors.

Another embodiment of the present invention is a method of modulating HCN channel gating. This method comprises providing to an HCN channel an effective amount of propofol or a propofol derivative having limited general anesthetic properties. Preferably, the HCN channel is an HCN1 channel.

As used herein, the term “modulate” with reference to HCN channel gating means significantly changing the opening and closing profile of the HCN channel in response to a stimuli, e.g., a ligand or voltage. For example, treatment of HCN channels with propofol or a propofol derivative of the present invention inhibits the opening of HCN channels in response to stimuli.

In one aspect of this embodiment, the propofol derivative comprises a compound of the formula (I):

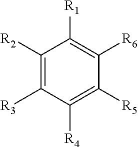

wherein
R₁ is selected from the group consisting of H and OH;
R₂, R₄, and R₆ are independently selected from the group consisting of H; —NR₁₀R₁₁, where R₁₀ and R₁₁ are independently selected from the group consisting of H and

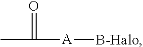

where A is a 5-membered heterocycle, B is a 6 membered aryl, and Halo is a halogen atom; C₁₋₄alkoxy; C₁₋₈alkyl, which is optionally substituted with one or more groups selected from the group consisting of —OH, —CF₃, carbonyl, —NH₂, and alkyne; C₁₋₄alkene optionally substituted with a 5- or 6-membered heterocycle, where from 0-2 carbon atoms of the heterocycle are optionally substituted with an atom selected from the group consisting of N, S, and O and one or more groups are pendant from a ring atom of the heterocycle, the pendant groups being independently selected from the group consisting of —H, —CH₃O, carbonyl, sulfonyl, —CH₃, —NH—OCH₃, and —CH₂CH₃; and —S—R₇—S—R₈ wherein R₇ is a C₁₋₄alkyl optionally substituted with C₁₋₄alkyl and R₈ is an aromatic ring optionally substituted with C₁₋₄alkyl or O—R₉, where R₉ is H or C₁₋₄alkyl optionally substituted with a carbonyl or —OH;
R₃ and R₅ are H;
or pharmaceutically acceptable salts thereof.

In another aspect of this embodiment, the propofol derivative is selected from the group consisting of:

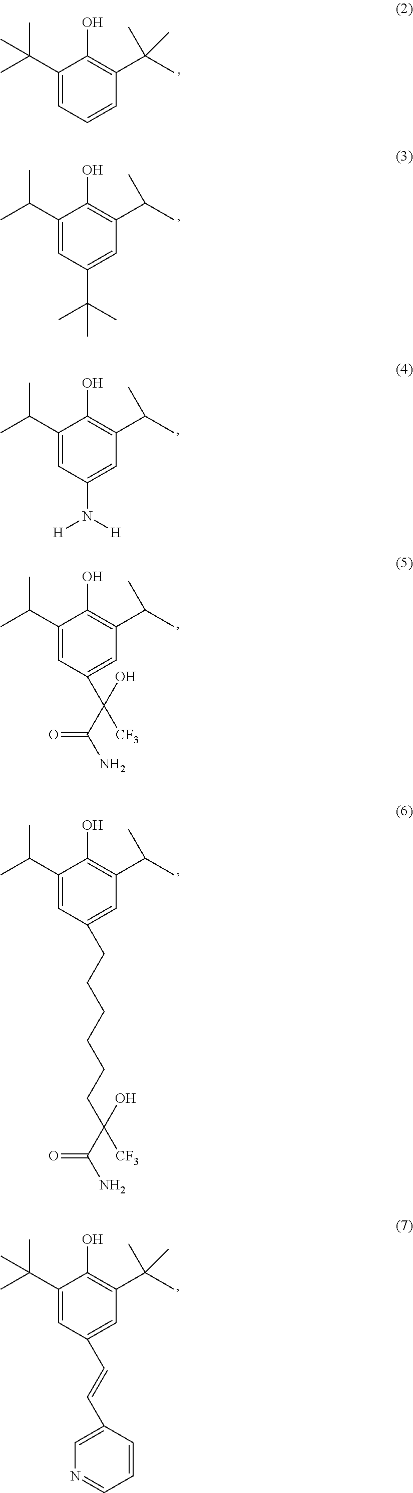

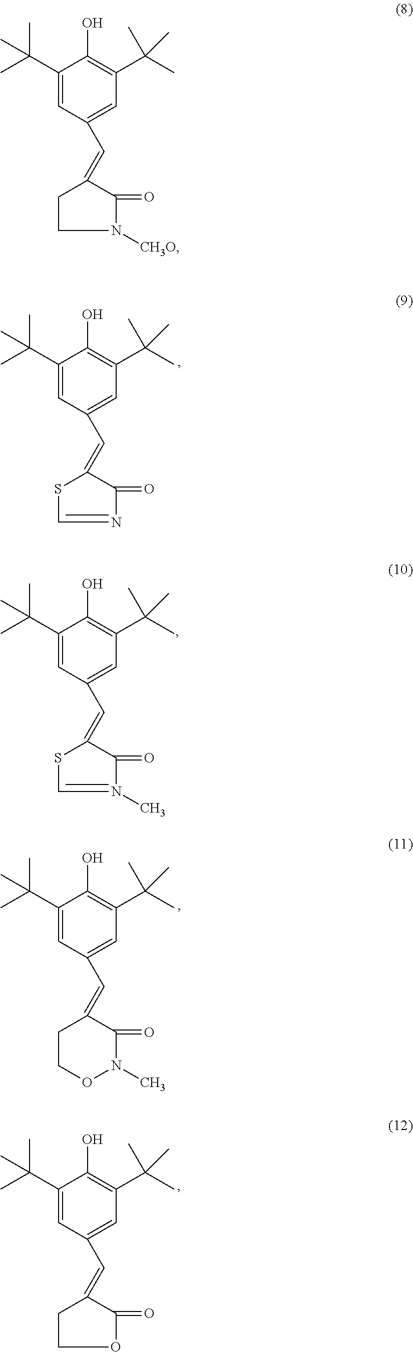

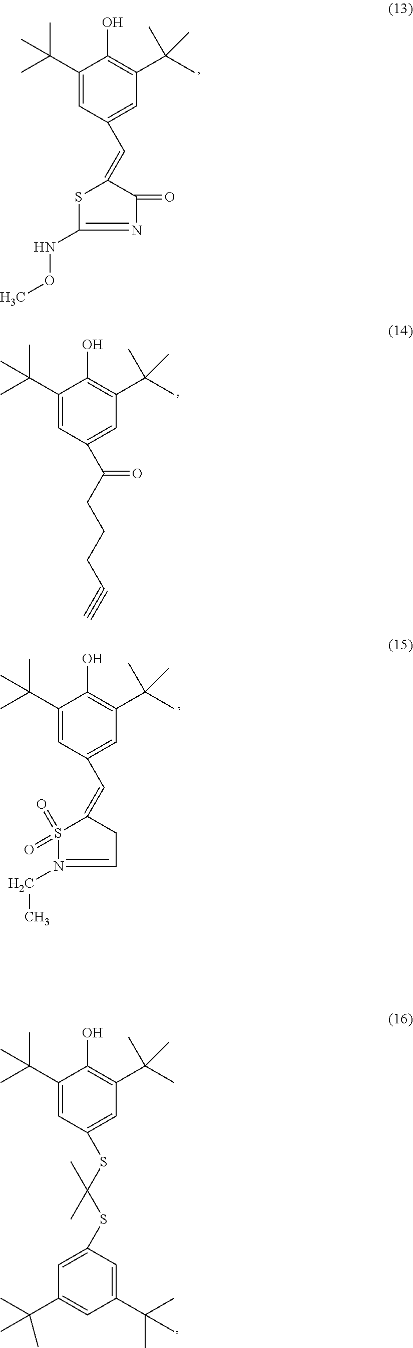

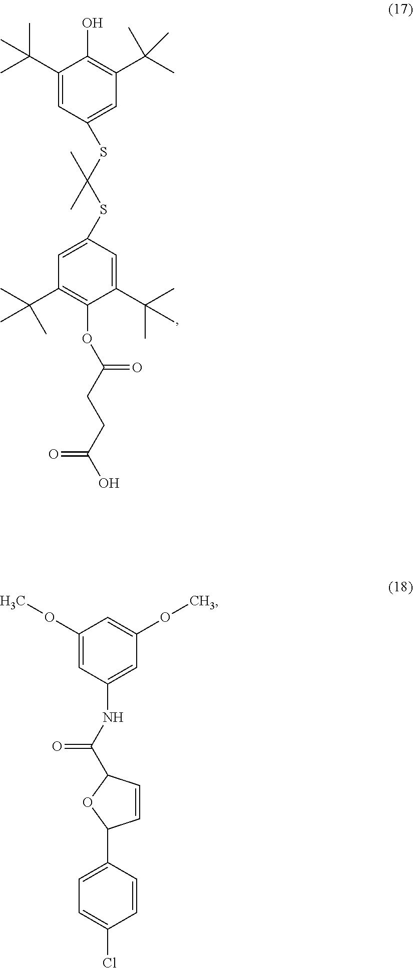

and pharmaceutically acceptable salts thereof.

In yet another aspect of this embodiment, the propofol derivative is selected from the group consisting of:

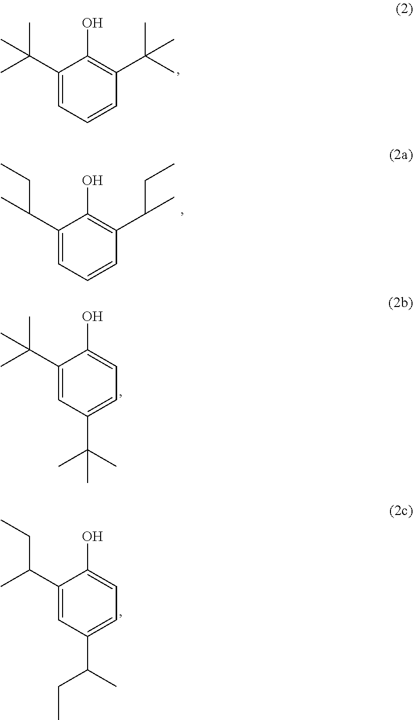

and pharmaceutically acceptable salts thereof. Preferably, the propofol derivative is

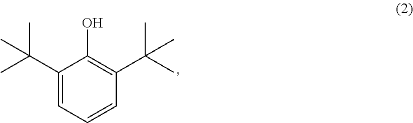

or a pharmaceutically acceptable salt thereof.

In a further aspect of this embodiment, the propofol derivative is selected from the group consisting of

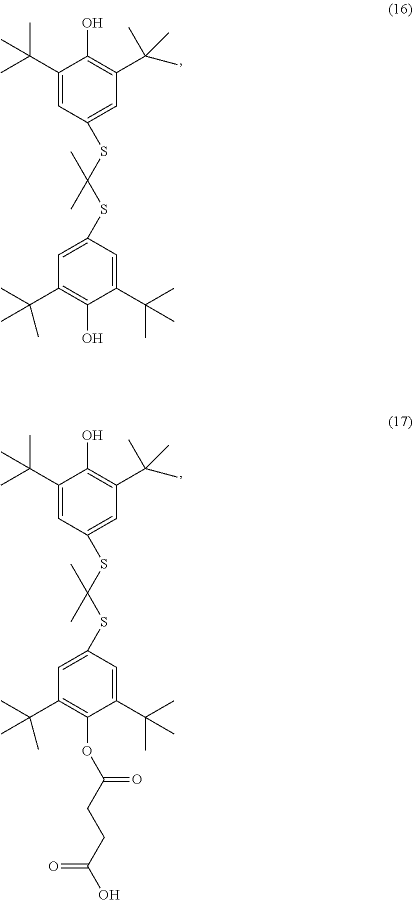

and pharmaceutically acceptable salts thereof.

In an additional aspect of this embodiment, the propofol derivative comprises a compound of the formula (II) or a compound of the formula (III):

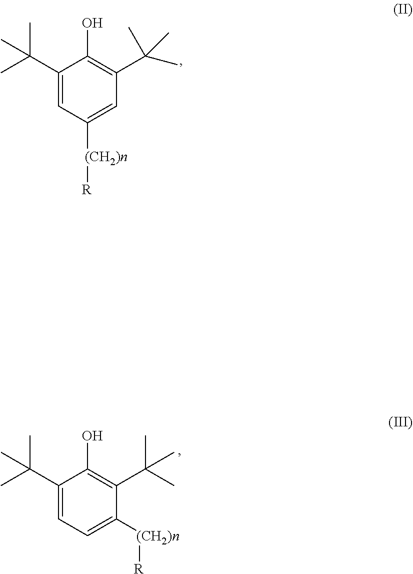

wherein n=2-36; and R is a positively charged group or atom;

or a pharmaceutically acceptable salt thereof. In this embodiment, n and R are further defined as set forth previously herein.

In yet another aspect of this embodiment, the propofol or propofol derivative is administered to a patient as part of a pharmaceutically acceptable composition. In this embodiment, the pharmaceutically acceptable composition is administered in a unit dosage form. The propofol or propofol derivative is present in the unit dosage form at a total concentration of e.g., about 1 μM to about 20 μM, although other concentrations may be used at the physician's option, based on, e.g., the patient's weight, age, etc., such that a non-hypnotic dosage is administered.

An additional embodiment of the present invention is a method of inhibiting an HCN1 channel without enhancing a gamma-aminobutyric acid-A (GABA-A) receptor comprising providing to an HCN channel an effective amount of a propofol derivative having limited general anesthetic properties.

As used herein, “enhancing” a GABA-A receptor means increasing the activation of a GABA-A receptor.

In one aspect of this embodiment, the propofol derivative comprises a compound of the formula (I):

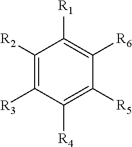

wherein
R₁ is selected from the group consisting of H and OH;
R₂, R₄, and R₆ are independently selected from the group consisting of H; —NR₁₀R₁₁, where R₁₀ and R₁₁ are independently selected from the group consisting of H and

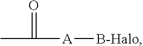

where A is a 5-membered heterocycle, B is a 6 membered aryl, and Halo is a halogen atom; C₁₋₄alkoxy; C₁₋₈alkyl, which is optionally substituted with one or more groups selected from the group consisting of —OH, —CF₃, carbonyl, —NH₂, and alkyne; C₁₋₄alkene optionally substituted with a 5- or 6-membered heterocycle, where from 0-2 carbon atoms of the heterocycle are optionally substituted with an atom selected from the group consisting of N, S, and O and one or more groups are pendant from a ring atom of the heterocycle, the pendant groups being independently selected from the group consisting of —H, —CH₃O, carbonyl, sulfonyl, —CH₃, —NH—OCH₃, and —CH₂CH₃; and —S—R₇—S—R₈ wherein R₇ is a C₁₋₄alkyl optionally substituted with C₁₋₄alkyl and R₈ is an aromatic ring optionally substituted with C₁₋₄alkyl or O—R₉, where R₉ is H or C₁₋₄alkyl optionally substituted with a carbonyl or —OH;
R₃ and R₅ are H;
or pharmaceutically acceptable salts thereof.

In another aspect of this embodiment, the propofol derivative is selected from the group consisting of:

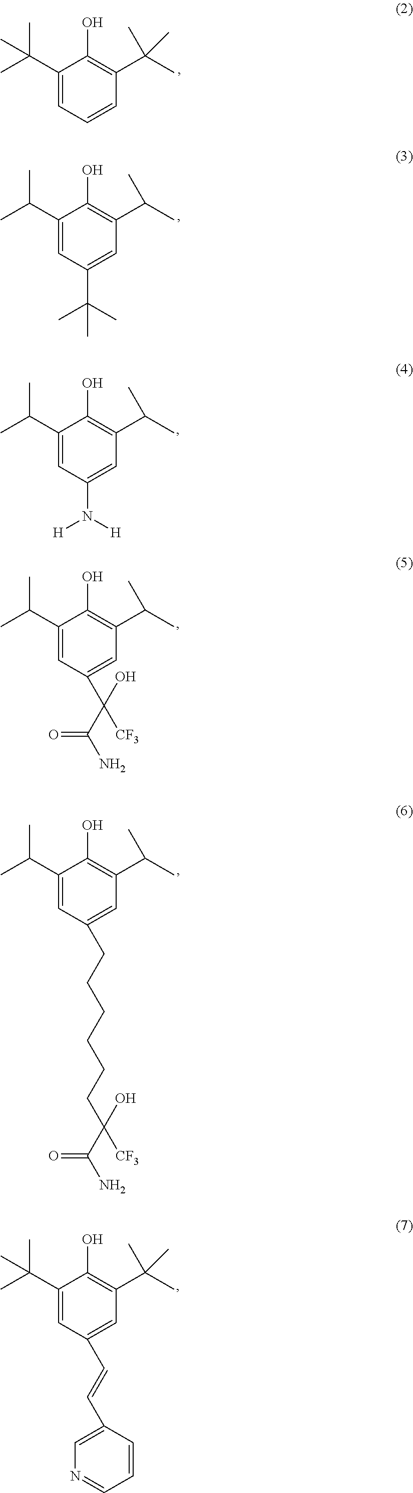

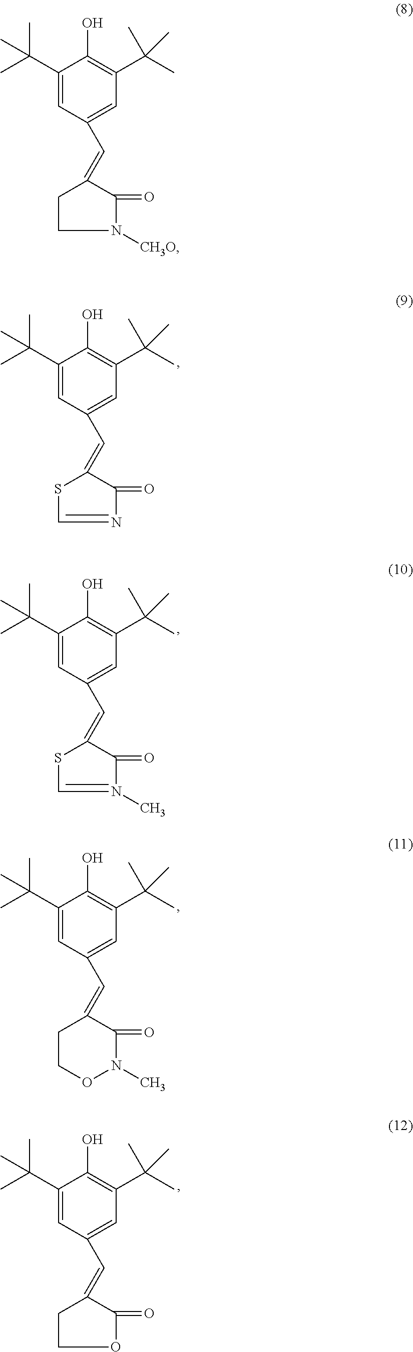

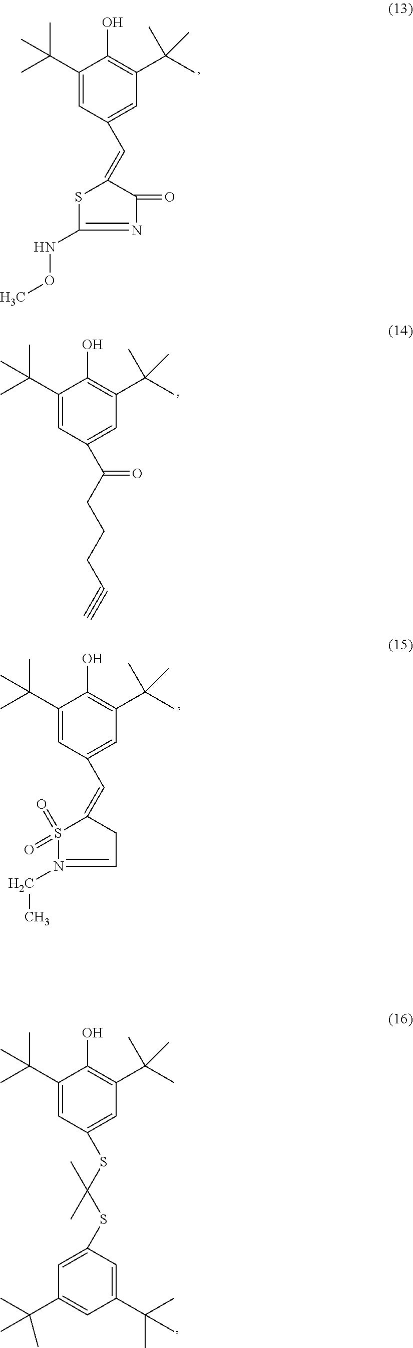

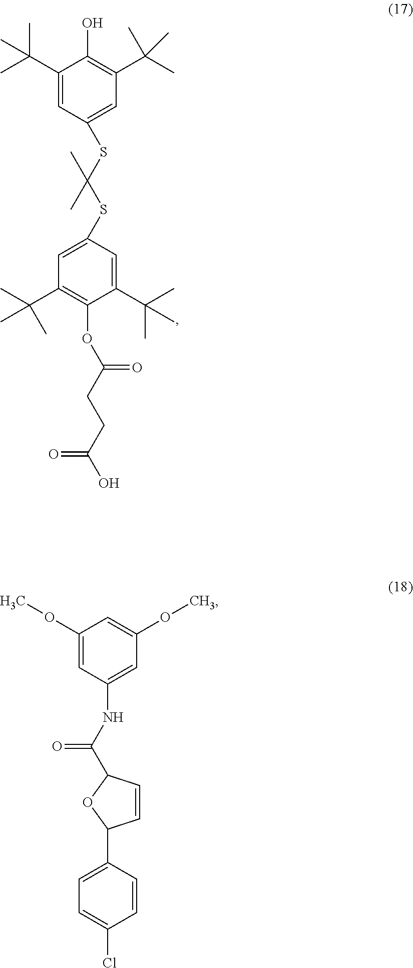

and pharmaceutically acceptable salts thereof.

In an additional aspect of this embodiment, the propofol derivative is

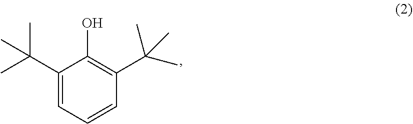

or a pharmaceutically acceptable salt thereof.

In a further aspect of this embodiment, the propofol derivative is selected from the group consisting of

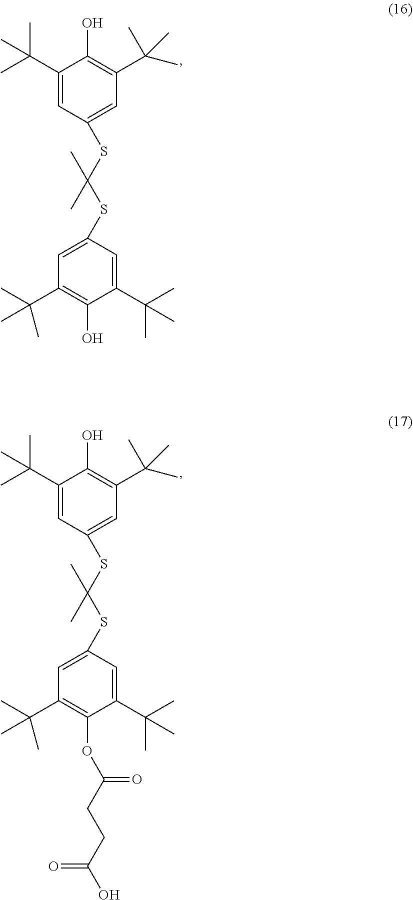

and pharmaceutically acceptable salts thereof.

In an additional aspect of this embodiment, the propofol derivative comprises a compound of the formula (II) or a compound of the formula (III):

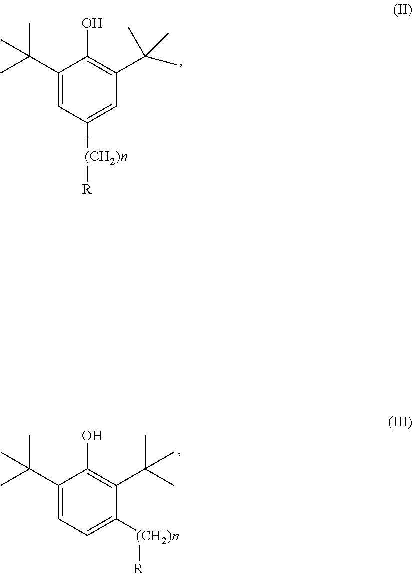

wherein n=2-36; and R is a positively charged group or atom;

or a pharmaceutically acceptable salt thereof.

In yet another aspect of this embodiment, the propofol derivative is administered to a patient as part of a pharmaceutically acceptable composition to manage or treat chronic pain in the patient. Preferably, the pharmaceutically acceptable composition is administered in an unit dosage form. As noted above, the propofol derivative is present in the unit dosage form at a total concentration of about 1 μM to about 20 μM.

In the present invention, an “effective amount” is an amount sufficient to effect beneficial or desired clinical results. An effective amount can be administered in one or more doses. In terms of treatment, an “effective amount” of a propofol or a propofol derivative is an amount sufficient to treat, manage, palliate, ameliorate, or stabilize, chronic pain or to modulate HCN channel gating, preferably HCN1 channel, gating, preferably, e.g., without inducing general anesthesia in a patient (I.e., a non-hypnotic dosage). Detection and measurement of these indicators of efficacy are discussed below.

The effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the drug being administered. For instance, the concentration of a propofol derivative need not be as high as that of propofol itself in order to be therapeutically effective.

In addition to the concentrations noted above and set forth in the claims, an effective amount of propofol or a propofol derivative is typically up to about 2% (weight/volume (w/v)) propofol based on the total weight of the selected dosage form, such as for example, up to about 3% (w/v) propofol derivative, including, for example, about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, and 2.9% (w/v) propofol or propofol derivative. More particularly, an “effective amount” delivers to the subject an amount that is below clinical doses used to induce general anesthesia (i.e., sub-hypnotic dosage levels), such as about 10 to about 200 μg/kg/min, preferably from about 50 to about 150 μg/kg/min, such as for example, about 60, 80, 100, 120 or 140 μg/kg/min of propofol or a propofol derivative according to the present invention.

Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of animal, and like factors well known in the arts of medicine and veterinary medicine. In general, a suitable dose of a propofol or a propofol derivative according to the invention will be that amount of the compound, which is the lowest dose effective to produce the desired effect. The effective dose of propofol or a propofol derivative maybe administered as two, three, four, five, six or more sub-doses, administered separately at appropriate intervals throughout the day.

Propofol or a propofol derivative may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, propofol or a propofol derivative may be administered in conjunction with other treatments. Propofol or a propofol derivative maybe encapsulated or otherwise protected against gastric or other secretions, if desired.

While it is possible for propofol or a propofol derivative of the invention to be administered alone, it is preferable to administer propofol or the propofol derivative as a pharmaceutical formulation (composition). The pharmaceutically acceptable compositions of the invention comprise propofol or one or more propofol derivative(s) as an active ingredient in admixture with one or more pharmaceutically-acceptable carriers and, optionally, one or more other compounds, drugs, ingredients and/or materials. Regardless of the route of administration selected, propofol or the propofol derivatives of the present invention are formulated into pharmaceutically-acceptable dosage forms, including unit dosage forms, by conventional methods known to those of skill in the art. See, e.g., Remington's Pharmaceutical Sciences (Mack Publishing Co., Easton, Pa.).

Pharmaceutical carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, salicylate, etc. Each carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen propofol derivative dosage form and method of administration can be determined using ordinary skill in the art.

The pharmaceutically acceptable compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen propofol derivative dosage form and method of administration may be determined using ordinary skill in the art.

Pharmaceutical formulations suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Formulations for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Formulations which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise propofol or one or more propofol derivatives in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In the foregoing embodiments, the following definitions apply.

The term “alkene”, as used herein, refers to an unsaturated aliphatic group containing at least one double bond.

The term “alkoxy” refers to an alkyl group, preferably a lower alkyl group, having an oxygen attached thereto. Representative alkoxy groups include methoxy, ethoxy, propoxy, isopropoxy, tert-butoxy and the like. Other alkoxy groups within the scope of the present invention include, for example, the following:

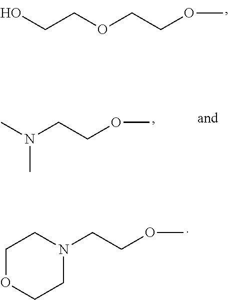

The term “alkyl” refers to the radical of saturated aliphatic groups, including straight-chain alkyl groups and branched-chain alkyl groups. In preferred embodiments, a straight chain or branched chain alkyl has 8 or fewer carbon atoms in its backbone (e.g., C₁-C₈ for straight chains, C₃-C₈ for branched chains).

The term “alkyne”, as used herein, refers to an aliphatic group containing at least one triple bond.

The terms “amine” and “amino” are art-recognized and refer to both unsubstituted and substituted amines and salts thereof, e.g., a moiety that can be represented by

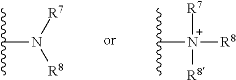

wherein R⁷, R⁸, and R^{8′} each independently represent a hydrogen or a hydrocarbyl group, or R⁷ and R⁸ taken together with the N atom to which they are attached complete a heterocycle having from 4 to 8 atoms in the ring structure. An “aromatic amino group” refers to an amino group in which the N atom is attached to at least one aromatic group.

The term “C_{x-y}” when used in conjunction with a chemical moiety, such as, alkyl, alkene, alkyne, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain.

The term “aryl” as used herein includes substituted or unsubstituted single-ring aromatic groups in which each atom of the ring is carbon. Preferably the ring is a 6-membered ring.

The term “carbonyl” as used herein refers to a functional group composed of a carbon atom double-bonded to an oxygen atom: C═O.

The term “guanidine” refers to the following functional group:

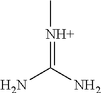

The terms “halogen” includes chloro, fluoro, bromo, and iodo.

The terms “heterocyclyl”, “heterocycle”, “heterocyclic”, and the like refer to substituted or unsubstituted aromatic or non-aromatic ring structures, preferably 5- to 6-membered rings, whose ring structures include no heteroatom, one heteroatoms, or two heteroatoms. The term “heteroatom” as used herein means an atom of any element other than carbon or hydrogen. Preferred heteroatoms are nitrogen, oxygen, and sulfur.

The term “quaternary ammonium group” is an amino group in which R⁷, R⁸, and R^{8′} each independently represent an alkyl group, or R⁷ and R⁸ taken together with the N atom to which they are attached complete a heterocycle having from 4 to 8 atoms in the ring structure.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), or a sulfonyl.

The term “sulfonyl” means a functional group obtained from a sulfonic acid by the removal of the hydroxyl group. Sulfonyl groups can be written as having the general formula R—S(═O)₂—R′, where there are two double bonds between the sulfur and oxygen.

It is understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers, optical isomers, and diastereomers.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that compounds of the present invention having a chiral center may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., “Enantiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

The following examples are provided to further illustrate the compositions and methods of the present invention. These examples are illustrative only and are not intended to limit the scope of the invention in any way.

### EXAMPLES

### Example 1—Materials and Methods

### Materials

General chemicals used in electrophysiology were of the highest purity from Sigma Aldrich (St. Louis, Mo.) as was 2,4-di-tert-butylphenol (2,4-DTBP) (137731), 2,6-di-tert-butylphenol (2,6-DTBP) (D48400) and DHβCD (H107).

For in vitro electrophysiological experiments, Propofol (TCI America, Portland, Oreg.), 2,6-DTBP, 2,6-Di-sec-butylphenol (ACROS 152830050—obtained via Fisher Scientific), 2,4-DTBP, and 2,4-Di-sec-butylphenol (S367907, Sigma Rare Chemicals Library) were normally dissolved in DMSO at 500 mM, and then diluted in the recording solution prior to use. In some experiments, 2,6-DTBP was dissolved in 40% 2-hydroxypropyl-beta-cyclodextrin at 1% (48.5 mM) and was diluted before use in recording solution to confirm that the 2-hydroxypropyl-beta-cyclodextrin solubilized compound (as used in animal experiments) had an equivalent efficacy to the DMSO solubilized material.

2-hydroxypropyl-beta-cyclodextrin was obtained from Sigma (H107-5G and C-0926 was used interchangeably without effect).

The two C₁₈-tethered quaternary ammonium derivatives of 2,6-DTBP (3C₁₈N-2,6-DTBP and 4C₁₈N-2,6-DTBP) will be synthesized commercially (Exclusive Chemistry, Obninsk, Russia).

Stock solutions of alkylphenols were stored at −20° C. for no more than one week while dilutions in recording solutions were prepared on the day of use.

For animal experiments, propofol was injected as the commercial and clinical 1% propofol formulation Diprivan® (1 propofol in intralipid, which contains: 10% soybean oil; 2.25% glycerol; 1.2% egg lecithin; 0.005% EDTA-Na₂ each as W/V, stored at 4° C., Abraxis Bioscience, Schaumburg, Ill.) while 2,6-DTBP was injected as inventors' own formulation wherein the drug was dissolved at 1% in 40% 2-hydroxypropyl-beta-cyclodextrin.

### Solubilization of Propofol and its Derivatives for In Vivo and In Vitro Analysis

For most in vitro experiments propofol and its dibutyl analogues were solubilized as 500 mM stocks in DMSO allowing for an averaged butylphenol density of 0.91 (Lorenc et al., 2003 and CAS/InCHEM data sheets). Serial dilutions were then dispersed into the aqueous recording solutions maintaining the DMSO concentration at 0.04% (W/V). Given that examined concentrations approach the aqueous solubility limits of the less soluble butyl compounds (variously reported as 20-200 μM for 2,6-di-tert-butylphenol versus propofol at 864-1740 μM (Trapani et al., 1996; Brewster et al., 1994; and CAS/InCHEM data sheets), recording solutions were shaken vigorously for 120 minutes after addition of propofol or its analogues. While it has been reported that 2,6-DTBP can be prepared as an injectable 1% (W/V) dispersion using a Cremaphor preparation (James et al., 1980), the inventors' initial attempts to solubilize 2,6-DTBP using the more biologically tolerated intralipid emulsion (akin to the Diprivan® formulation of propofol (Baker et al., 2005)) were unsuccessful. Thus, it was found that even if the 2,6-DTBP was liquefied by heating to 60° C. prior to addition of intralipid and the mixture shaken vigorously for 1-18 hours, the compound was largely recovered as a yellow oily accumulation on top of the white intralipid emulsion. In contrast, it was found that 2,6-DTBP could be solubilized at 1% (W/V) in a carrier solution of 40% (W/V) DHβCD (a biologically tolerated carrier (Brewster et al., 1990)) using a simple, standardized, protocol. To this end, appropriate masses of 2,6-DTBP and DHβCD that would yield 6 ml of 1% 2,6-DTBP, in combination with 40% DHβCD, were combined in a Teflon® capped borosilicate vial and heated to 60° C. in a water bath. This temperature is above the melting point of 2,6-DTBP (36° C. (Lorenc et al., 2003) but below that of DHβCD (>200° C. (Loftsson et al., 1996) and is compatible with stability of both reagents (Lorenc et al., 2003; Loftsson et al., 1996 and CAS documentation). After 15 minutes, the molten 2,6-DTBP was intimately mixed with the DHβCD by vigorous vortexing, 200 μl of 60° C. DI H₂O added and, following additional vortexing, the vial returned to the water bath for 15-30 minutes. Following addition of 3 more 200 μl aliquots of deionized water (DI H₂O), with corresponding incubation and vortexing, the sample was left at 60° C. overnight. The resulting colorless viscous gel was slowly diluted by addition of the required volume of DI H₂O adding no more than 200 μl at a time with 15-30 minutes incubation at 60° C. and vortexing after each addition. The resulting clear, colorless, solution could be stored at −20° C. with no phase separation or derivative precipitation. Visual observation upon dispersal of this co-solution into an aqueous phase was consistent with aqueous solvation rather than precipitation of the 2,6-DTBP. If DHβCD were omitted, after the overnight regime 2,6-DTBP was present as yellow oily droplets akin to those observed when the compound was dispersed in the presence of intralipid.

### Gas Chromatography Analysis of 2,6-DTBP

Gas chromatography analysis of 2,6-DTBP was performed according to a modification of a method for propofol analysis (Yu et al., 1993; Fan et al., 1995) using a Shimadzu GC-2010 Plus gas chromatograph (Shimadzu, Kyoto, Japan) equipped with a split/splitless injection unit, a forensic crossbond phase fused silica column (Rtx-BAC2, 25 m×0.53 mm, 2 μm film thickness, Restek U.S., Bellefonte, Pa.) and a flame ionization detector (FID). The injection port and FID were at 280° C. while the column temperature was 210° C. The split ratio was 2:1 while the column flow was 6.96 ml/min with helium (linear velocity 65 cm/s).

Samples and standards were prepared as follows. To 200 μl of whole blood (drawn following injection of DHβCD or 2,6-DTBP solubilized in DHβCD), 200 μl of chloroform containing 100 nM thymol (Sigma) as internal standard and 2 μl of DMSO were added. Following mixing (vortex for 20 seconds followed by 30 minutes on a rotator) and subsequent centrifugation (5000×g for 10 minutes at 10° C.), the chloroform phase was transferred into a glass gas chromatography vial and washed with an equal volume of 0.5 M NaOH. To compare 2,6-DTBP acutely solubilized in DMSO with 2,6-DTBP solvated in DHβCD, an aliquot of the appropriate solution containing 10 nmoles of 2,6-DTBP was combined with 200 μl chloroform containing the thymol internal standard (an addition that would result in a concentration of 50 μM 2,6-DTBP in the chloroform phase if all the 2,6-DTBP partitions therein). DHβCD was added to the DMSO-solubilized 2,6-DTBP sample and DMSO was added to the DHβCD-solubilized 2,6-DTBP sample so that the final concentrations of DHβCD and DMSO were equivalent. An equal volume of H₂O was added, the samples were mixed vigorously, and then the chloroform phase was recovered as described above. A control wherein 2,6-DTBP was omitted was processed in parallel.

For the standard calibration curve, aliquots of 2,6-DTBP acutely solubilized in DMSO were added to 200 μl of chloroform plus the thymol internal standard (total DMSO addition was 2 μl in all cases). 200 μl of H₂O were then added and the samples were processed as described above. In all cases, 2 μl of the chloroform phase was injected for analysis.

### Methods

Certain of the methods and modeling are as in Lyashchenko et al., 2007. Other methods are set forth in more detail below.

### Animal Procedures

Xenopus oocytes were harvested from frogs anesthetized by immersion in ice-cold pH 7 buffered (sodium phosphate) 0.05% Tricane according to a Columbia University IACUC approved protocol (PI#366G CU#2928). Oocytes were maintained in L-15 media (Specialty Media).

### Surgical Procedures and Drug Administration.

Briefly, while under anesthesia, a small incision was made in the left and right thighs of mice and the sciatic nerve exposed. The ipsilateral nerve was partially ligated while the contralateral nerve was mechanically manipulated in a manner similar to that done on the ipsilateral side but no ligation performed. The animals were sutured then allowed to recover for 5-7 days. This surgical technique is the commonly used Selzer model for induction of neuropathic pain.

Adult female C57 Black/6J mice (16 to 24 weeks old) were subject to partial sciatic nerve ligation (“PNL”) (Malmberg et al, 1998; Seltzer et al, 1990) according to institutional and Federal guidelines. In brief, following induction of isoflurane (2.5-3%) anesthesia, surgical incisions were made in the upper aspect of both the left and right hind limbs. In the left limb, approximately one half to two thirds of the nerve was ligated using an 8.0 braided silk suture (SofSilk, Coviden, Mansfield, Mass.). The nerve in the right limb was subject to similar mechanical manipulation but no suture applied. 21 of 24 C57 mice recovered from the procedure. Post-PNL behavioral testing was not begun until at least seven days after surgery.

Although the surviving mice tended to exhibit protective behavior towards the ipsilateral paw, their behavior was otherwise normal. Accordingly, and in keeping with the approved protocol, no therapeutic agents other than the indicated experimental test compounds were administered.

To examine 2,6-DTBP bioavailability, 32 mice received dihydroxy-β-cyclodextrin (DHβCD) solubilized 2,6-DTBP (see below), DHβCD alone or saline by intraperitoneal (i.p.) injection with a dosing schedule as per behavioral testing (see below). 10-60 minutes after receipt of the appropriate final dose, mice were anesthetized with 2.5-3% isoflurane then exsanguinated by venous puncture. Blood concentration of 2,6-DTBP was determined by gas chromatography (see below). To consider toxicity of a high acute therapeutic dose of 2,6-DTBP, some animals received a single bolus of 80 mg/kg. To consider toxicity of an acute hyper-therapeutic dose of 2,6-DTBP, all animals that received three 40 mg/kg therapeutic doses during animal behavior experiments received a 120 mg/kg bolus immediately after the 180 minutes of behavioral testing window, thereby raising the cumulative dose to 240 mg/kg. The general condition of all of these animals was followed by qualitative observation of behavior (grooming and exploration) and survival. All animals were sacrificed within 7 days after receipt of a 2,6-DTBP/DHβCD injection.

### Animal Behavior.

Mice were analyzed with respect to their sensation of painful stimuli using accepted behavioral tests. Briefly, the response of application of a heat lamp to the hind paws was used to determine sensitivity to thermal stimuli (to assay thermal hyperalgesia) and the application of calibrated fibres to hind paws to determine sensitivity to mechanical stimuli (to assay mechanical allodynia). During these tests the animals were awake and behaving freely albeit within a restricted space to facilitate execution of the experiment. In the thermal sensitivity assay, the time it takes before the animal withdraws its paw from the heat source (hind paw withdrawal latency, HPWL) was taken as the measure of thermal sensitivity. In the mechanical assay, the number of times the animal lifts its paw clear of the fiber in 10 trial applications is taken as the measure of mechanical sensitivity.

To monitor the sensitivity of ipsilateral and contralateral hind paws to mechanical and thermal insults, mice were subjected to Von Frey fiber and thermal latency stimulus-response analysis using calibrated Von Frey fibers and a timed, tightly focused, variable intensity infrared heat source (all obtained from IITC Life Science Inc., Woodland Hills, Calif.) as described previously (Udesky et al., 2005; Rowley et al., 2008). Briefly, for each test, an animal was separately placed within a 132 cm² open-topped clear Plexiglass corral on the appropriate surface (a plastic-coated grid with a mesh size of 6 mm or a clear glass thermally regulated platform for the Von Frey and latency analyses, respectively) to which they had been previously acclimated (placement in the corrals without testing for extended sojourns during at least one week pre trial) and allowed to settle for at least 30 minutes. Tests were performed on the plantar surface of both left and right hind paws. In the Von Frey analysis, the probability of paw withdrawal (P_{W}) was obtained by determining how many of 10 trials with a particular fiber resulted in the tested paw being withdrawn. Ipsilateral and contralateral paws were sequentially tested with a single fiber with fiber strengths of 0.05, 0.4, 0.6, 1.1, 2.5, 3.3 and 4 g tested from weakest to strongest. In the thermal sensitivity analysis, the mean hind paw withdrawal latency (HPWL) was obtained by averaging the latency observed in five separate trials at a particular setting of the heat source. HPWL tests of the ipsilateral and contralateral paws were interleaved. The response to heat source settings of 3 to 30% (in 3% increments) were tested in a random order. If the paw was not withdrawn within 30 seconds the trial was terminated and a latency of 30 seconds noted.

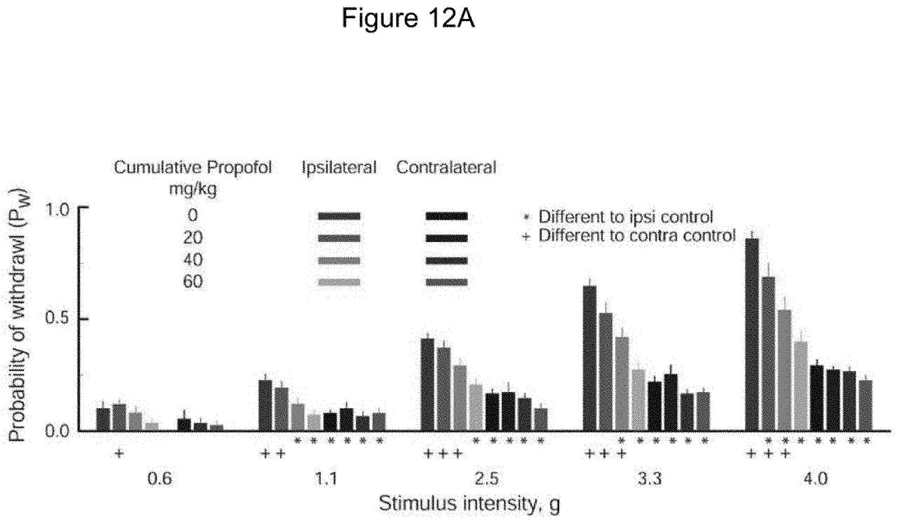

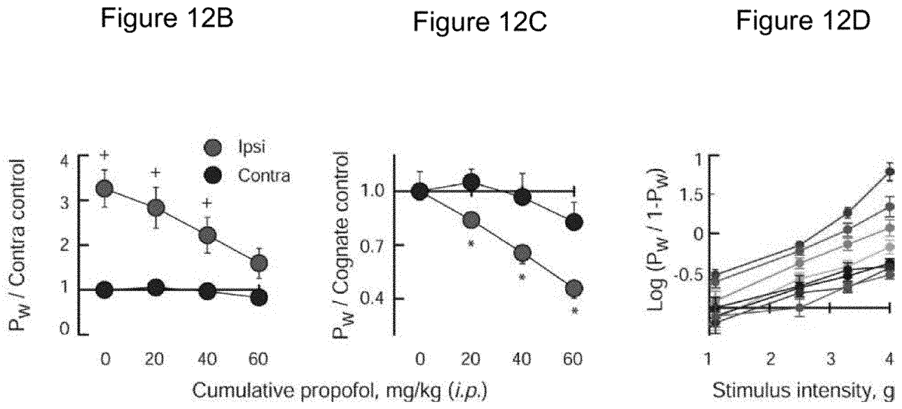

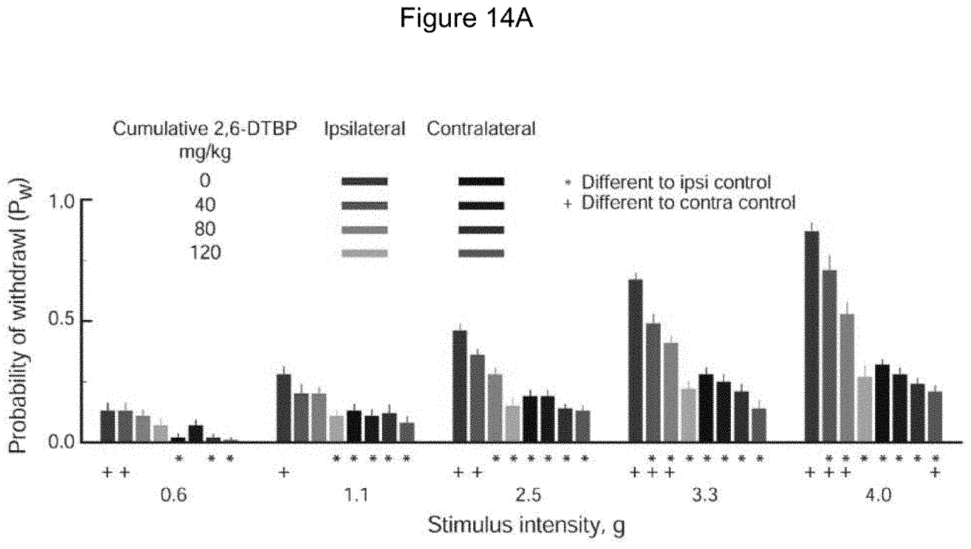

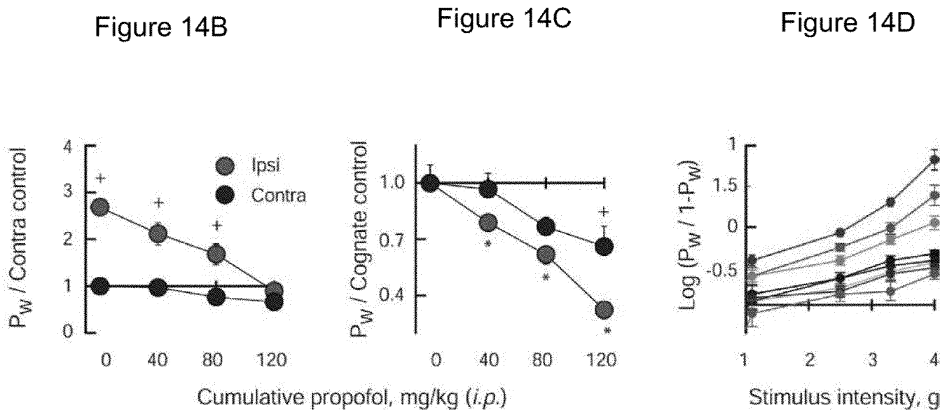

Von Frey fibers exerting 2.5, 3.3 and 4 g yielded optimal discrimination of mechanical neuropathic hyperalgesia with respect to nociception. Thus, these fibers tended to produce withdrawal probabilities greater than 0 in the contralateral paw but less than 1 in the ipsilateral paw (FIGS. 12A and 14A) while the ratio of P_{W IPSI}/P_{W CONTRA} was similar at each stimulus intensity (2.9±0.3, 2.9±0.4 and 2.7±0.2, respectively). Accordingly, the mean values of P_{W} ratios over these three stimuli was used to obtain a single better defined value of the ratio for each mouse at each time point and drug condition. To examine mechanical allodynia, a Log it transformation of the mean withdrawal probabilities was performed.

A heat lamp setting of 15% was found to be optimal for detection of the thermal hyperalgesia with respect to nociception. Thus, latencies in both ipsilateral and contralateral paws fell between the fastest and slowest detection thresholds (some 2-3 and 30 seconds, respectively) while the ratio HPWL_{IPSI}/HPWL_{CONTRA} was lowest at 15%. In contrast, 30% stimulation produced a largely, albeit not completely, nociceptive response in either injured and uninjured paws thereby monitoring the basal reflex.

Based on the above observations, an objective set of inclusion criteria was established to identify animals that developed mechanical and thermal neuropathic responses. For an animal to be included in the Von Frey mechanical analysis, control values yielding P_{W IPSI}/P_{W CONTRA}>1 and P_{W IPSI} at 4 g≥0.3 were required while for inclusion in the HPWL thermal analysis the control HPWL_{IPSI}/HPWL_{CONTRA} at 15% had to be ≤0.8. Based on these criteria, 18 of the 21 mice that recovered from PNL surgery developed robust and stable mechanical and thermal neuropathic responses. Of the other 3 animals, one exhibited an early robust phenotype that was extinguished after two weeks while two failed to develop any overt neuropathic phenotype. No data from the two unresponsive mice were included in the findings presented here. The early results obtained from the mouse that showed a late spontaneous decline in its neuropathic phenotype were included, but data obtained after week 2 were eliminated.

To examine the influence of agents on nocifensive behavior, cumulative dose response relationships were constructed wherein an aliquot of agent was administered every 60 minutes.

Following each i.p. injection, animals were allowed to recover for 10 minutes then their behavior assayed during the subsequent 50 minutes. To maximize the sensitivity of the assay while minimizing the duration of the injection/monitoring cycles, and hence of drug clearance, only lamp settings of 15 and 30% and fiber strengths of 0.6, 1.1, 2.5, 3.3 and 4 g were examined during such drug trials. The reported dose of administered agents assumes no clearance of compound during the analysis.

Sedation and motor coordination may be assessed using open field and rotarod testing as described in Cheng et al. (2006).

### Molecular Biology

cDNA encoding murine (HCN1, 2 and 4) and human (HCN3) HCN channels were subcloned into pGH19 (HCN1 and 4) or pGHE (HCN2 and HCN3) vectors and amplified in STBL2 cells (Invitrogen Corporation, Carlsbad, Calif.). cRNA was transcribed from NheI (HCN1, HCN3 and HCN4) or SphI (HCN2) linearized DNA using T7 RNA polymerase (Message Machine; Ambion, Houston, Tex.). 11-50 ng RNA was injected into each Xenopus oocyte.

### Electrophysiology:

Recordings were made from Xenopus oocytes 2-5 days after cRNA injection. Cells were maintained in L-15 media without ficoll (Specialty Media, Phillipsburg, N.J.) at 17° C. until use.

Two microelectrode voltage clamp (TEVC) data, acquired using a Warner Instruments (Hamden, Conn.) OC-725C amplifier, were recorded with Pulse software (HEKA Elektronik, Lambrecht/Pfalz, Germany) following filtering of the wide bandwidth output of the clamp at 2.5 kHz (with a Frequency Devices 902 8-pole Bessel filter) and digitization at 5 kHz using an ITC-18 interface (Instrutech Corporation, Port Washington, N.Y.).

Microelectrodes were fabricated from 1B120-F4 borosilicate glass (World Precision Instruments, Sarasota, Fla.) and had resistances of 0.1-0.5 MΩ (I passing) and 1-4 MΩ (V sensing) when filled with 3 M KCl. The Ag—AgCl ground wire(s) of the active virtual ground circuit were connected to the bath solution by 3M KCl-2% agar salt bridges placed downstream of, but close to, the oocyte. Recordings were obtained at room temperature (22-24° C.). In all cases the observed potential was within 1% of the reported command potential. For HCN1 and 2, oocytes were bathed in a recording solution of (in mM) 107 NaCl, 5 KCl, 2 MgCl₂, 10 HEPES-free acid pH 7.4 (NaOH). For the more poorly expressing HCN3 and 4 channels, the KCl concentration was raised to 25 mM by isoosmolar substitution of NaCl. In all recordings, the holding potential was −30 mV and the tail potential 0 mV.

To analyze the effects of propofol derivatives on HCN channel gating, isochronal activation curves were constructed. In brief, cells were placed in 20 ml glass scintillation vials (containing 15 ml of recording solution that was, where indicated, supplemented with vehicle or compound) and incubated at room temperature on a 3-D rotator (Lab Line, Melrose Park, Ill.). After 20 minutes, cells were transferred to a recording chamber continuously perfused with the appropriate drug, vehicle or control solution. In some experiments, isochronal activation curves were recorded before and after incubation in the presence or absence of drug or vehicle.

Channels were activated by hyperpolarizing steps applied in −10 mV intervals for 5 (HCN1), 30 (HCN2) or 60 seconds (HCN3 and 4). The amplitude of the instantaneous tail currents following each sweep was determined as the difference between the plateau current (observed after the voltage-clamp has settled and the uncompensated linear capacitance decayed but before marked channel closure) and the baseline current (observed after deactivation was complete).

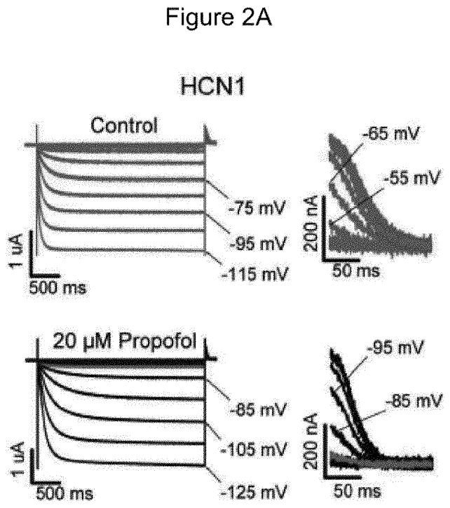

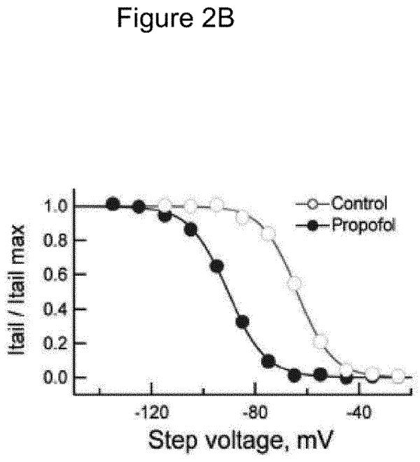

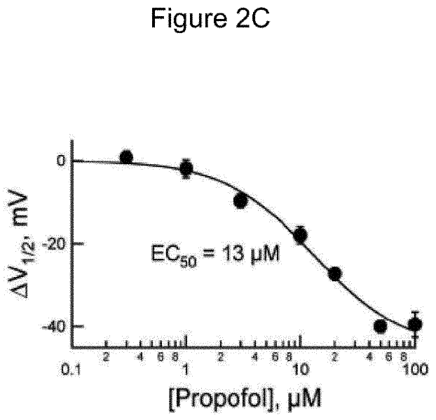

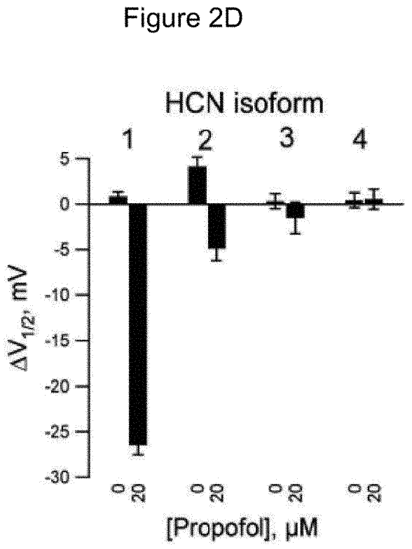

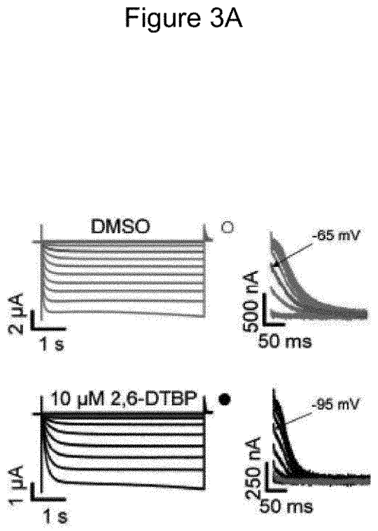

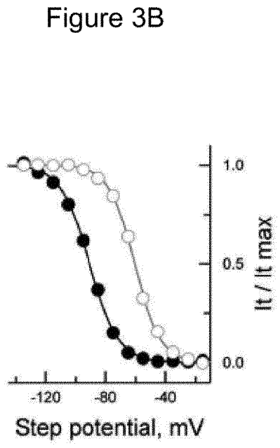

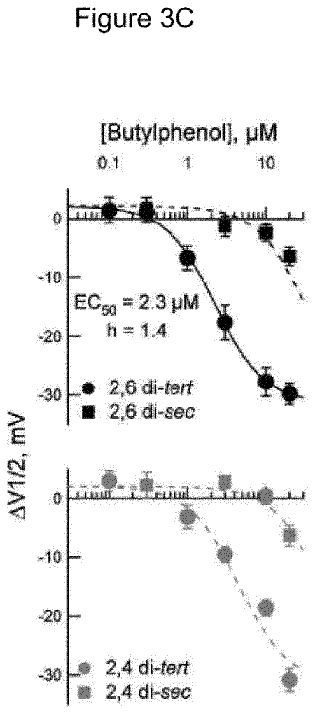

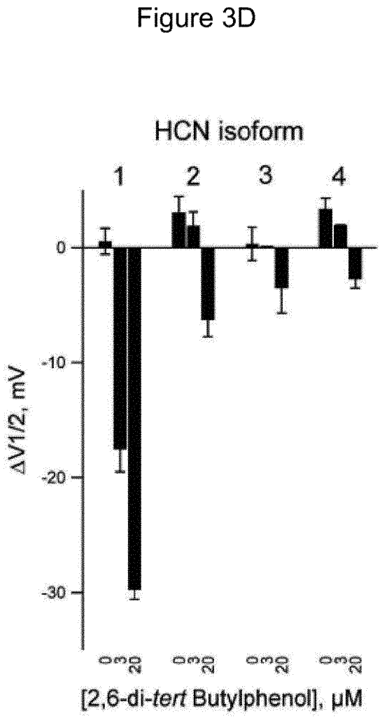

Channel activation and deactivation were monitored in response to changes in the membrane voltage. Shown in FIGS. 2A-2D and 3A-3D are representative steady-state activation curves (FIGS. 2B and 3B) and kinetic responses of the channel as a function of time at each voltage (FIGS. 2A and 3A). These effects were quantified by plotting the amplitude of the tail currents (right hand records in FIGS. 2A and 3A) then obtaining the midpoint potential and slope of the relationship by fitting the relationship with the Boltzmann function (as shown in FIGS. 2B and 3B). Such experiments were then performed in the presence of varying concentrations of propofol or modified versions thereof, namely, propofol derivatives according to the present invention, and the change in the behavior of the channels observed. In FIGS. 2A-2D and 3A-3D, the response of HCN1 channels to propofol and 2,6-di-tert-butylphenol are shown in the upper and lower sections of Panels A, respectively. FIGS. 2B and 3B show the tails current amplitudes and Boltzmann fits of the records shown in the respective FIGS. 2A and 3A and reveal that the presence of propofol and 2,6-di-tert-butylphenol leads to a negative shift in gating with respect to that observed in the absence of drug. Such experiments were performed for channels formed from each of the HCN isoforms (or heteroassemblies thereof) and the effects of propofol and its derivatives on these different channels determined in the same manner as described above (results from such experiments shown in FIGS. 2C-2D and FIGS. 3C-3D).

Changes in the kinetic and equilibrium responses of the channels can then be interpreted in terms of models of how the channels function in the presence and absence of varying concentrations of the drug. From such analyses the mechanism of action of the drug on the channel can be deduced.

Based on the collected data, gating models to describe the behavior of the channels in the presence and absence of the drug were formalized. By fitting such models to the data, the mechanism of action of the drug was deduced. Such analysis may be carried out in programs such as IgorPro and MatLab.

### Paradigms and Analysis:

Steady-state gating parameters were determined by fitting tail current activation curves with the Boltzmann equation, I(V)=A₁+A₂/{1+exp[(V−V_{1/2})/s]}, where A₁ is the current offset, A₂ the maximal amplitude, V is step voltage, V_{1/2} is the activation mid-point voltage and s is the slope factor. Tail amplitudes and Boltzmann fits were normalized to the maximal tail amplitude, A₂−A₁ for display. After determination of an initial activation curve, cells were transferred to a 20 ml glass scintillation vial containing L-15 and allowed to recover for 10 minutes. Following drug, vehicle or control incubations (20 minutes in recording solution on a 3-D rotator from Lab Line, Melrose Park, Ill.), cells were then transferred back to the recording chamber and the post-treatment activation curve determined in the continuous presence of the appropriate drug, vehicle or control solution. No cell was exposed to more than one condition.

To follow changes in channel activation with better temporal resolution than could be achieved from serial determinations of complete activation curves, equation 2 was used to determine ΔV_{1/2 APPARENT} from records collected with a two-step protocol.

ΔV_{1/2 APP}=−s*Ln {[1−G_{R}]/[G_{R}*exp(V_{INT}/s)−exp(V_{MAX}/s)]}−V_{1/2} (2)

Here, G_{R} is the conductance ratio (G_{INT TAIL}/G_{MAX TAIL} or G_{INT}/G_{MAX}) determined from currents (I_{INT} and I_{MAX}) recorded at V_{INT} and V_{MAX} (step potentials that elicit partial and saturating levels of activation) and V_{1/2} and s are the initially-determined activation mid-point and slope (see Fogle et al., 2007 for details). Analysis of G_{INT TAIL}/G_{MAX TAIL} or G_{INT}/G_{MAX} yielded equivalent estimations of ΔV_{1/2 APP}.

Dose-response data were fit using the Hill equation (3) where R is the response, R_{MAX} is the maximal response, [A] is the ligand concentration, EC₅₀ is the ligand concentration that produces a half-maximal response, and h is the Hill coefficient.

R/R_{MAX}=1/[1+(EC₅₀/[A])^{h}] (3)

### Data and Statistical Analysis:

Data and statistical analysis were performed in PulseFit (HEKA Elektronik), using custom analysis routines written in IgorPro (Wavemetrics Corporation) or Excel (Microsoft Corporation, Redmond, Wash.). Statistical analysis was performed using one-way ANOVA with post hoc Holm-Sidak analysis (enabled in SigmaStat V3.1, Systat Software, Point Richmond, Calif.). Unless a reference population was defined, tests were performed across all possible pairs and relevant pairs identified from the matrix post hoc. All data are presented as mean±SEM. For normalized data, the error around the denominator is that factor's observed error divided by its observed mean. Student's t-tests, Student's paired t-tests and one and two way ANOVA with post hoc Holm-Sidak analysis were used as appropriate.

### Example 2—Propofol, at Therapeutically Acceptable Concentrations, Powerfully and Selectively Antagonizes the Function of HCN1 Channels

The principal effects of propofol on wild type (wt) HCN1 are recapitulated in FIGS. 2A-2D. Inspection of the TEVC current families and the corresponding tail currents obtained in the absence and presence of 20 μM propofol showed that the drug slows opening, accelerates closing and shifts gating to more hyperpolarized potentials. A fit of the Hill equation to the results from a number of such experiments shows the apparent affinity (EC₅₀ about 7-13 μM) and maximal efficacy (ΔV_{1/2} of −35 to −45 mV) of the propofol-mediated hyperpolarization of gating (see also Cacheaux et al., 2005).

As previously reported, the slower and more cAMP sensitive HCN2 and HCN4 channels were, respectively, weakly and almost completely insensitive to the anesthetic.

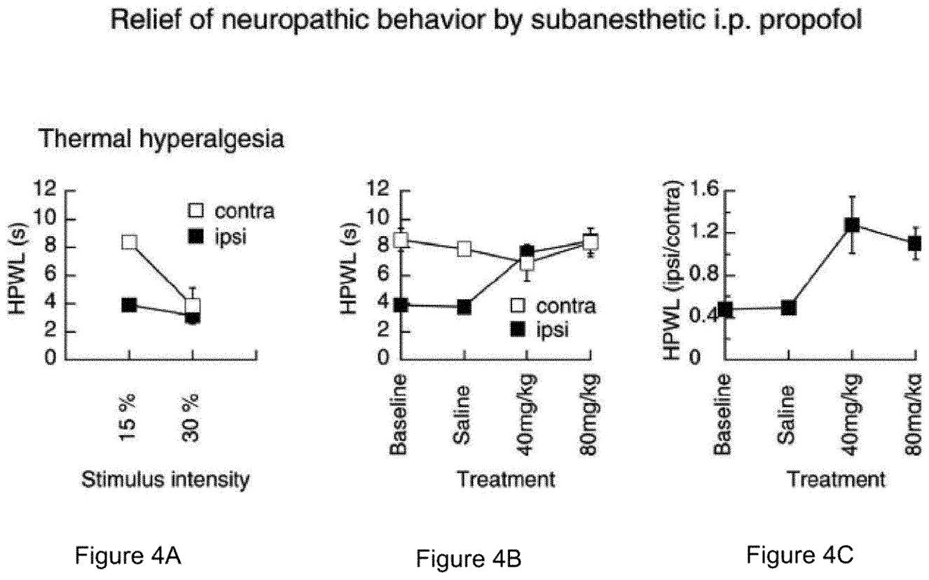

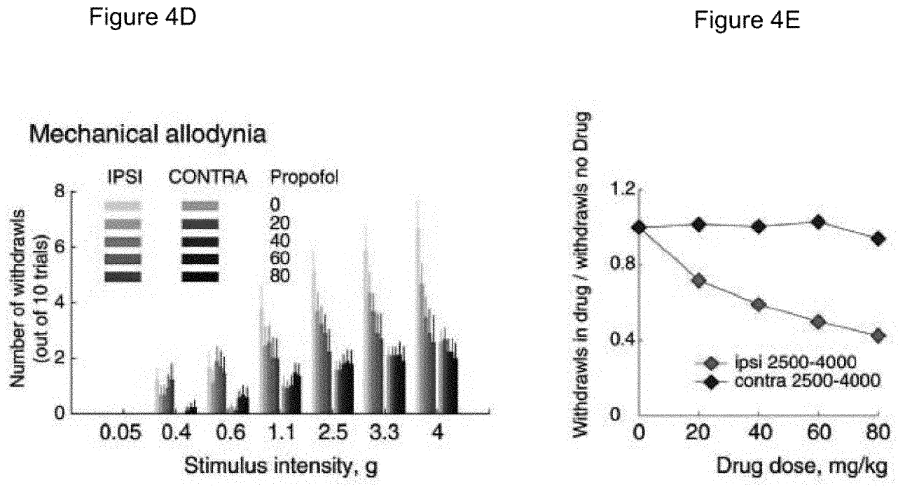

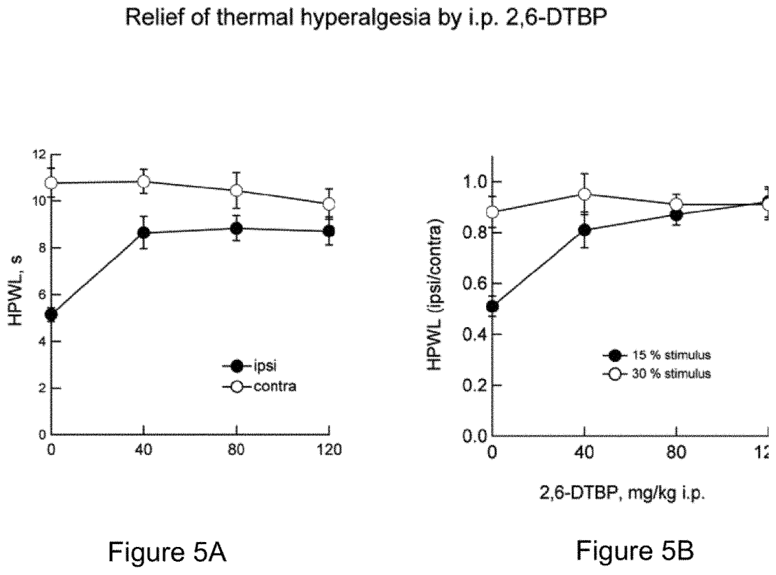

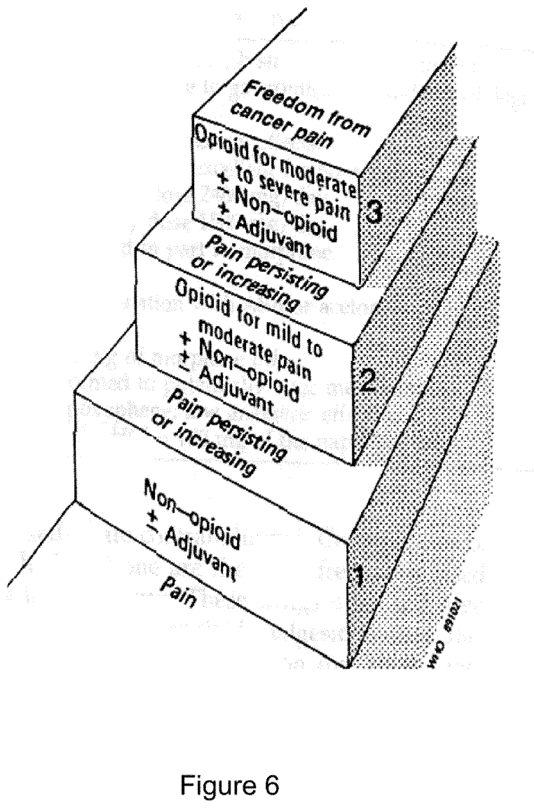

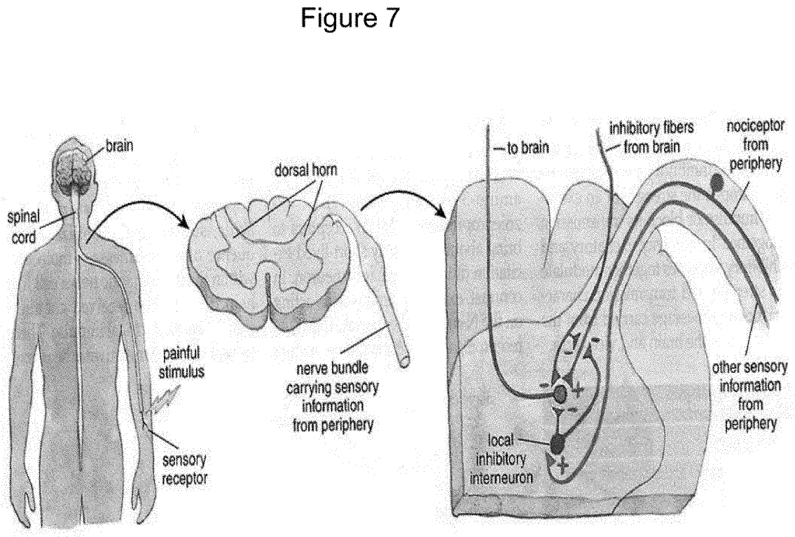

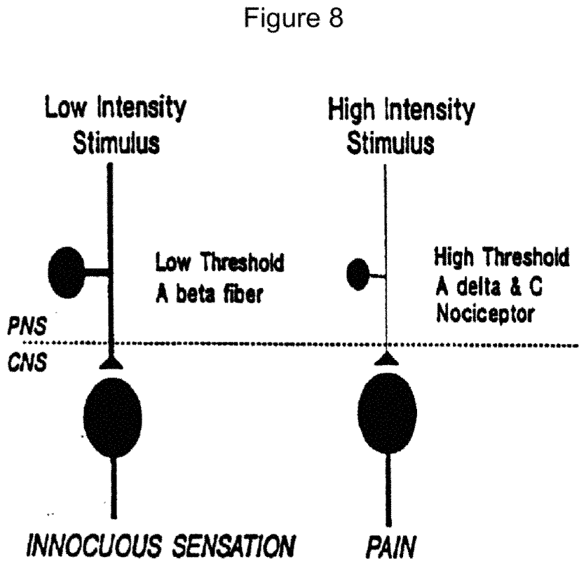

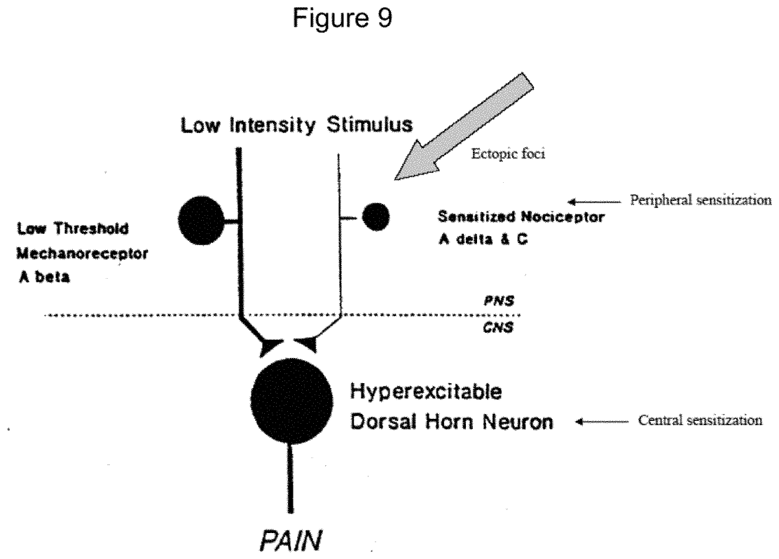

With reference to FIGS. 4A-4E, the experiments showed that subanesthetic propofol alleviates at least two modalities of neuropathic pain.

### Example 3—Subhypnotic Doses of Propofol Reduce Mechanical Allodynia and Mechanical and Thermal Hyperalgesia

FIG. 12A shows P_{W IPSI} and P_{W CONTRA} in post-PNL mice as a function of stimulus strength (Von Frey fibers ranging from 0.6 to 4 g) and i.p. propofol administration (cumulative dose of propofol ranging from 0 to 60 mg/kg). These doses of propofol markedly reduced the mechanical hyperalgesia observed in the ipsilateral paw with only modest disturbance of normal mechanical nociceptive response as monitored in the contralateral paw. P_{W IPSI} was significantly higher than P_{W CONTRA} in the absence of propofol, but the difference was significantly reduced upon administration of 20 mg/kg propofol and, at a dose of 60 mg/kg, the response becomes indistinguishable from the control value of P_{W CONTRA}. In contrast, 20 to 60 mg/kg propofol had no statistically significant effect on P_{W CONTRA} suggesting that in the absence of neuropathy, mechanical nociceptive reflexes are relatively insensitive to propofol (consistent with an earlier study (Udesky et al., 2005)).

In FIGS. 12B and 12C, P_{W IPSI} and P_{W CONTRA} were plotted as a function of the contralateral control value (FIG. 12B) and their respective values in the absence of drug (FIG. 12C). In each case, the ratios determined across the 2.5 to 4 g stimulus strengths were averaged (see Example 1 above). FIG. 12B shows that subhypnotic propofol provided about 75% reversal of the neuropathic phenotype (the ratio dropped from 3.3 to 1.6 where unity would represent complete amelioration). FIG. 12C shows this benefit comes with almost complete selectivity with respect to maintenance of nociceptive processing. Thus, while there was a trend towards reduced sensitivity at 60 mg/kg propofol, this decrease (unlike the neuropathic relief) did not reach statistical significance. No animals lost their righting reflex consistent with previous demonstrations that greater than 100 mg/kg i.p. propofol is required for obviation of this reflex (Udesky et al., 2005; Lingamaneni et al., 2001). Based upon these values, selective suppression of neuropathic mechanical hyperalgesia occurs at doses 3-10-fold lower than hypnosis.

An important aspect of clinical neuropathic pain is the emergence of mechanical allodynia, a sensitivity to previously innocuous touch. While the sampling paradigms were not optimized to examine low intensities, a Log it transformation of the population mean of the mechanical data suggested subhypnotic propofol selectively restored the mechanical threshold to that observed in the uninjured paw (FIG. 12D with threshold set at a P_{W} of 0.1).

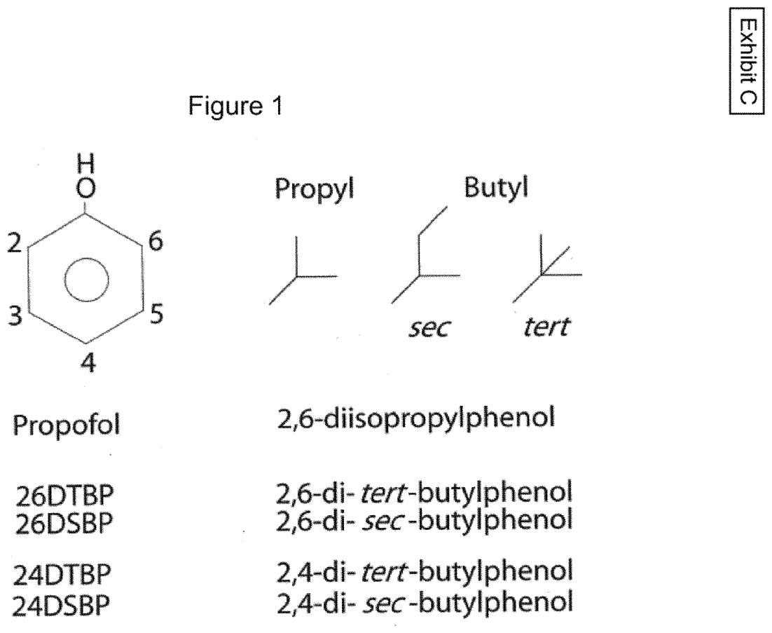

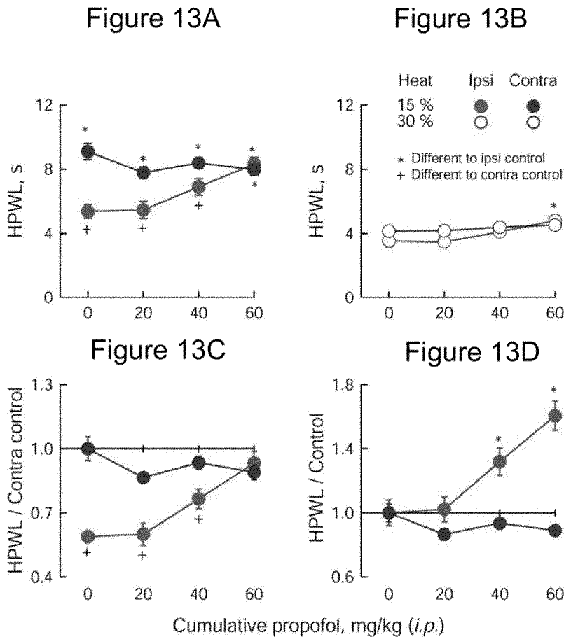

FIGS. 13A-13D show the assessment of thermal hyperalgesia analyzed in a manner equivalent to that shown in FIG. 1 for mechanical hyperalgesia. Thus, FIGS. 13A and 13B plot HPWL_{IPSI} and HPWL_{CONTRA} values at stimulus strengths of 15% and 30%, respectively (each as a function of the cumulative i.p. propofol dose) while FIGS. 13C and 13D report these latencies as a function of the contralateral control value (FIG. 13C) and their cognate zero drug condition (FIG. 13D). As observed with mechanical hyperalgesia, subhypnotic doses of propofol powerfully and preferentially relieved neuropathic thermal hyperalgesia.

### Example 4—2,6 Di-Tert-Butylphenol is a Potent Antagonist of HCN1 Channel Gating

Modification of the alkyl adducts on propofol (e.g., replacement of propyl groups by butyl groups) yielded tertiary butyl compounds that retained efficacy as antagonists of HCN channels with the parental propofol preference for channels formed from HCN1. (FIGS. 3A-3D) These tertiary butyl compounds have previously been shown to be ineffective against GABA-A channels and are non-anesthetic. The butyl derivatives of propofol were solubilized in the injectable carrier compound, 2-hydroxy-propyl-beta-cyclodextrin.

FIG. 3A shows TEVC recordings from Xenopus oocytes expressing homomeric HCN1 channels (Left) and the corresponding tail currents (FIG. 3A, Right) obtained following incubation with either DMSO alone (Top) or 10 μM 2,6-DTBP (Bottom). In all four panels, the red line highlights the trace recorded with an activation voltage of −65 mV. Qualitative inspection of these data suggested that 2,6-DTBP makes HCN1 channels harder to either activate and/or open. Thus, the drug appeared to slow opening, accelerate closing and shift gating to more hyperpolarized potentials. Plots of the equilibrium activation relationships for these two recordings confirmed the later observation (FIG. 3B). Such findings were qualitatively identical to the effects of propofol (Cacheaux et al., 2005).

From recordings such as those shown in FIGS. 3A and 3B, a concentration response relationship was constructed for modulation of the V_{1/2} of HCN1 gating by 2,6-DTBP (FIG. 3C Top—data fit by the solid line). These data revealed that inhibition of channel gating by 2,6-DTBP is about 5-6 times more potent than that by propofol (EC₅₀ of 2.3 μM versus 13 μM) but it acts with a similar maximal efficacy (ΔV_{1/2} of −32 mV versus −45 mV) and apparent stoichiometry (h of 1.4 versus 1.1) when the effects of the drugs are determined under equivalent experimental conditions (Lyashchenko et al., 2007). Importantly, 2,6-DTBP displayed a similar efficacy irrespective of whether it was initially solubilized in DMSO or DHβCD (HCN1 ΔV_{1/2} after 20-minute incubation with 20 μM 2,6-DTBP was −31.9 mV±0.7, n=6 versus −27.7 mV±0.7, n=6 for DMSO and DHβCD, respectively).

To gain insight into whether 2,6-DTBP markedly alters the behavior of fully activated channels, the maximal current density (as obtained from fits of the Boltzmann equation to equilibrium activation curves) was determined before and after incubation in the presence or absence of 2,6-DTBP or vehicle alone. In all cases, the post incubation current was larger than the pre incubation current, but this run up appeared to be modestly blunted in the presence of 2,6-DTBP (Control: 121±4, n=8; DHβCD: 124%±7, n=7; 20 μM 2,6-DTBP: 108%±7, n=6; albeit this apparent difference did not reach statistical significance). Two tentative conclusions may be drawn from these data. First, the generalized run up in current amplitude was likely to be due to forward translocation of vesicular channels into the plasma membrane during the room temperature incubation. Second, the primary effect of 2,6-DTBP appeared similar to that of propofol, specifically it acts to stabilize the closed/deactivated state of HCN1 channels with little effect as a blocker of the conduction path (Lyashchenko et al., 2007).

### Example 5—Alkylphenol Association with HCN1 Channels is Mediated Via a Sterically Defined Site Whose Structural Constraints are Inverted with Respect to the Propofol Site of GABA_{A} Receptors

To further examine the nature of the selective alkylphenol association with HCN1 channels, whether coupling of HCN1 channels to butylphenols displayed any dependence on the architecture or location of the alkyl adducts attached to the phenol ring was determined. Specifically, the ability of 2,6-di-sec- as well as 2,4-di-tert- and 2,4-di-sec-butylphenols to inhibit HCN1 channel gating was examined. In addition to the inhibition-response relation for 2,6-DTBP, FIG. 3C plots the inhibition-response relations for 2,6-DSBP (Top) and 2,4-DSBP and 2,4-DTBP (Bottom). In each case, the dashed lines through the respective data represent the fit of the Hill equation determined for 2,6-DTBP offset by 2, 15 and 23-fold (for 2,4-DTBP, 2,6-DSBP and 2,4-DSBP, respectively). These data revealed that there was a strong preference for the tertiary versus the secondary arrangement of the butyl side chains (by about 12-15-fold) and a preference (albeit weaker) for 2,6- over 2,4-substitutions (about 2-fold). Remarkably, the preference for the tertiary arrangement of the butyl groups was the exact inverse of the steric requirements that control alkylphenol association with GABA_{A}-Rs. It should be noted that as higher concentrations appeared to exceed the aqueous solubility limit under the experimental conditions (by visual inspection—see also Example 1 above), it is possible the selectivity for di-tert over di-sec may be stronger than is reflected here.

That is, the maximal efficacy as well as potency may have been reduced.

### Example 6—2,6 Di-Tert-Butylphenol Selectively Antagonizes Gating of HCN1

As judged by its effects on the V_{1/2} (and lack of effect on maximal current), propofol is an efficacious and potent antagonist of HCN1 channel gating that exhibits marked selectivity with respect to the other HCN isoforms (ΔV_{1/2} by 20 μM propofol is −9.1 mV±1.6 n=6, −0.6 mV±0.1 n=6 and 0.1 mV±1.4 n=4 for HCN2, 3 and 4, respectively—(Lyashchenko et al., 2007 and previously unpublished data). FIG. 3D shows that 2,6-DTBP retains this selectivity profile.

### Example 7—2,6-Di-Tert-Butylphenol Reverses the Neuropathic Responses to Mechanical and Thermal Insults while Leaving Nociception Largely Intact

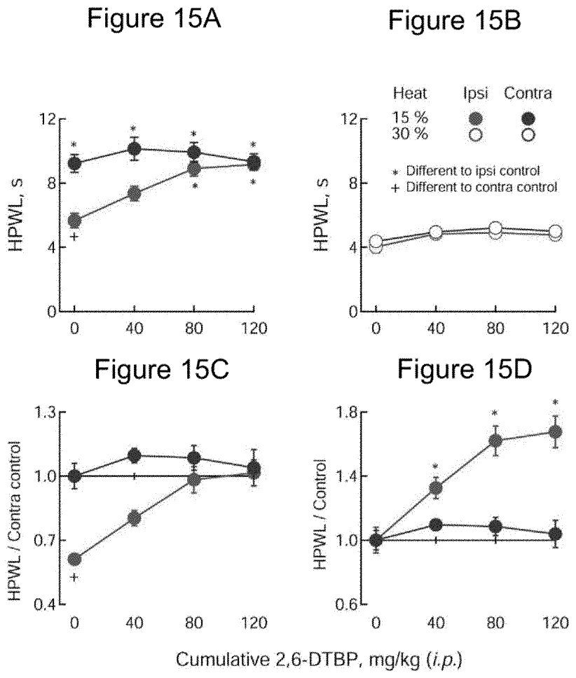

Tertiary butyl substitution abolishes coupling of 2,6-alkylphenol to GABA_{A}-Rs (an effect that is manifested physiologically as a total absence of anesthetic efficacy of 2,6-DTBP at serum levels equivalent to those that are fully hypnotic for propofol) but leaves alkylphenol inhibition of HCN channels intact. Thus, if recruitment of the inhibitory ligand gated channels represents a critical aspect of propofol's efficacy as a neuropathic pain selective analgesic, 2,6-DTBP should be largely or completely ineffective. In contrast, if HCN1 containing I_{H} channels are involved in the neuropathic analgesic activity of alkylphenols, 2,6-DTBP would retain a propofol-like efficacy. FIGS. 14A-14D and 15A-15D show that 2,6-DTBP ameliorates the behavioral consequences of PNL-induced hyperalgesia with respect to both mechanical (FIGS. 14A-14D) and thermal insults (FIGS. 15A-15D) and does so with a selective retention of nociceptive transmission that closely mirrors the activity of propofol. As observed with subhypnotic propofol, a Log it transformation of the population mean of the mechanical data suggests 2,6-DTBP acts to restore selectively the mechanical threshold to that observed in the uninjured paw. These therapeutic effects are not due to vehicle (data not shown).

Thus, the antineuropathic effect of 2,6-di-tert-butylphenol suggests it or other non-anesthetic analogues may represent a novel class of drugs that could be effective in mitigating neuropathic pain.

### Example 8—Thermal Stability, Bioavailability and Toxicity of DHβCD Solubilized 2,6-DTBP

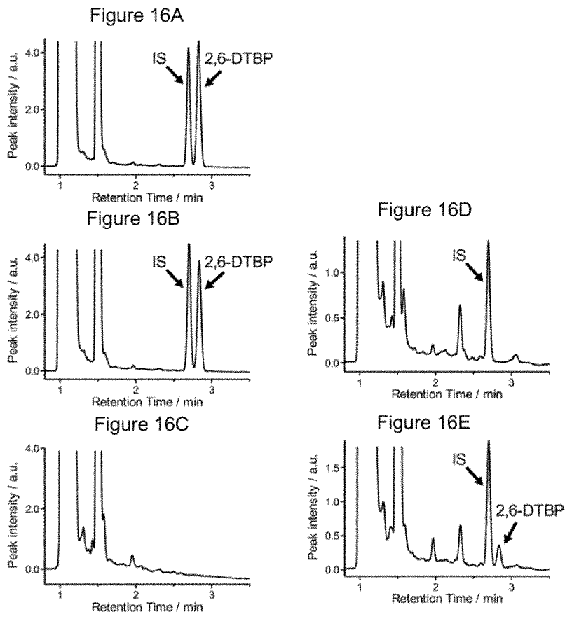

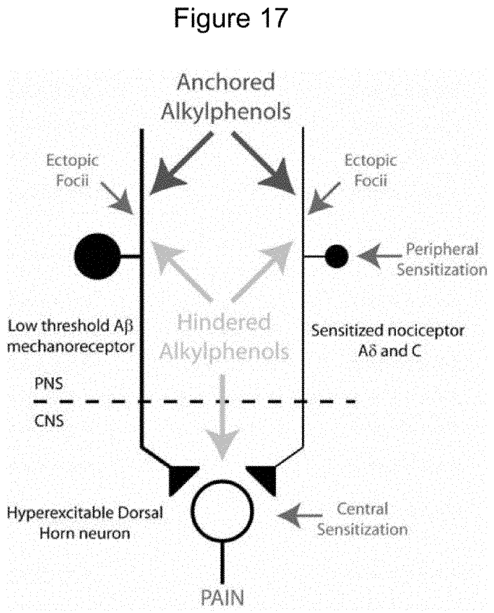

To generate an injectable formulation of 2,6-DTBP, a paradigm was developed that solvated the alkylphenol in complex with an aqueous soluble excipient, DHβCD. Because this process took 18 hours at 60° C. and alkylphenols are subject to oxidation, there is a possibility that significant degradation of the 2,6-DTBP may occur during preparation (Hassanein et al., 1994). This could lead to an underestimation of the potency of 2,6-DTBP or misassigning it a bioactivity that actually resides with a degradation product. However, we speculated the actual loss was likely to be markedly less given the protective effect of compound caging within a cyclodextrin structure (Brewster et al., 1994; Loftsson et al., 1996). In accord with this hypothesis, gas chromatography chromatograms obtained from analysis of 2,6-DTBP dissolved in DMSO immediately prior to analysis (FIG. 16A) or following solvation in DHβCD according to the protocol described in Example 1 (FIG. 16B) were essentially identical with no evidence of additional peaks (compare also to FIG. 16C wherein vehicles without 2,6-DTBP were analyzed—note that the thymol standard was omitted here for clarity).

Whether DHβCD solvated 2,6-DTBP is readily bioavailable and what are the blood concentrations during the behavioral testing window were addressed by injecting animals with increasing doses of the standard 2,6-DTBP/DHβCD formulation or DHβCD alone and recovering whole blood by venous puncture. The samples were then processed for gas chromatography as described in Example 1. FIGS. 16D and 16E show chromatograms obtained from samples wherein animals had received DHβCD or 80 mg/kg 2,6-DTBP. Injection of 2,6-DTBP results in emergence of a peak at the characteristic retention time of the alkylphenol. The whole blood concentration of 2,6-DTBP indicated from this analysis was 3.6 μM (10.6 μM±4.9, mean±SEM, n=6). Such an observation accords with i.p. DHβCD solvated 2,6-DTBP having similar pharmacokinetic and pharmacodynamic behavior as i.p. propofol.

It has previously been reported that 2,6-DTBP is well tolerated following acute exposure to intravenous doses of 80-100 mg/kg (James et al., 1980). Although the absolute dose is roughly comparable to that used in the behavioral studies, the blood concentration achieved through intravenous injection is likely to be some 40-fold greater than that achieved in the behavioral tests. Such findings suggest 2,6-DTBP is not acutely toxic. To gain further insight into toxicity associated with the elevated systemic load consequent upon use of i.p. injection, two trials were performed. First, four mice were exposed to a single bolus dose of 80 mg/kg, a dose which appears to offer a near maximal therapeutic relief. In agreement with the findings of James and Glen (1980), all mice tolerated this dose well. Thus, they survived for the seven day post-2,6-DTBP observation window, during which time their behavior was indistinguishable from paired controls. Second, at the end of the cumulative dose response behavioral studies (that is after administration of three 40 mg/kg doses over a 180-minute window), mice were injected with a bolus dose of 120 mg/kg 2,6-DTBP. Unlike the first three doses, this large final dose was not well tolerated. Thus, all animals displayed a marked lethargy that was not anesthetic in its nature and which included labored respiration, arching of the back, suppression of exploration and trembling. Moreover, while 9 out of 32 animals recovered and survived out to the 7 day end point, the remaining animals died over a fairly narrow window of 14-18 hours. Toxicity is unlikely to be related to the excipient, DHβCD, as it was found that animals tolerated equivalent doses well and this accords with the findings of others (Brewster et al., 1990; Rajewski et al., 1996).

Alkylphenols are subject to oxidation (Hassanein et al., 1994) yielding, for example, quinones or dimerized DTB hydroxy/quinones (Bedell et al., 1983; Wang et al., 1984; Fujiyama et al., 1999) and such compounds are biologically available and bioactive (e.g. probucol and succinobucol (Stocker et al., 2009; Wasserman et al., 2003)). However, the observations that DHβCD and DMSO solubilized 2,6-DTBP, are equally effective as antagonists of HCN1, and are chemically equivalent as examined by gas chromatography suggests there is little or no loss or modification of 2,6-DTBP during its solubilization in the presence of the excipient, DHβCD. This is consistent with DHβCD exerting a protective effect by caging the alkylphenol within the cyclodextrin structure (Brewster et al., 1994; Loftsson et al., 1996). Clearly, these results do not formally exclude the possibility that a low abundance contaminant of the 2,6-DTBP accounts for the biologically relevant analgesic activity, but they argue against such a compound arising during formulation.

If the DHβCD and intralipid vehicles result in markedly different pharmacokinetic and pharmacodynamic behaviors, the relative efficacy of the agents may be poorly reflected in the behavioral data. It is not believed that this is the case for the following reasons. First, the pharmacokinetics and pharmacodynamics of propofol solvated in DHβCD are indistinguishable from propofol dispersed in intralipid or Cremaphor, supporting the notion that DHβCD will function as a reliable vehicle for in vivo dispersal of the butylphenol analogues (Egan et al., 2003; Trapani et al., 1998; Viernstein et al., 1993). Second, the whole blood concentrations of 2,6-DTBP observed are those one would expect to see if propofol and 2,6-DTBP have similar pharmacokinetic properties irrespective of vehicle. Third, DHβCD solubilized 2,6-DTBP is comparable to 2,6-DTBP solubilized in DMSO as an HCN1 antagonist supports the notion that the caged compound is readily aqueous and available upon dilution. Fourth, as 2,6-DTBP is observed to achieve essentially full efficacy as a neuropathic analgesic, the key effect of a dispersal error would be to underestimate its potency and not yield a false positive.

The therapeutic index of i.p. 2,6-DTBP appears to be on the order of 3 to 5. Thus, a therapeutically beneficial acute dose of 40 to 80 mg/kg is well tolerated both immediately after injection and over the subsequent seven days of observation. However, an acute dose of 240 mg/kg appears to be close to the LD₇₅. Similar results were obtained when 2,6-DTBP was administered intravenously (James et al., 1980). Based on the pharmacokinetics of propofol, it is likely that the effective analgesic plasma concentration of alkylphenols could be achieved, at least transiently, with a 40-fold lower dose when the drug is administered intravenously (in mice, the intravenous dose of propofol for anesthesia is 5 mg/kg (James et al., 1980) while intraperitoneal administration requires a 40-fold higher load—(Udesky et al., 2005; Lingamaneni et al., 2001). Intravenous administration of 2,6-DTBP would thus be anticipated to have a acute therapeutic index closer to 120 to 200.

The finding that two closely related alkylphenols, the intravenous anesthetic propofol and its non-anesthetic congener, 2,6-DTBP, both preferentially inhibit heterologously expressed HCN1 channels and selectively provide analgesia for neuropathic pain is consistent with HCN1 as the presumptive therapeutic target for this class of compounds.

During general anesthesia, the steady-state free concentration of propofol in the plasma is on the order of 1 μM (Table 2 below). As neuropathic pain analgesia appears to emerge at 3 to 10 fold lower doses than this ED₅₀ for anesthesia, it is estimated that analgesia is evoked by serum free concentrations of the order of 0.1 to 0.3 μM. Assuming that propofol and 2,6-DTBP partition similarly and proportionally, it is estimated that an analgesic dose of 2,6-DTBP of 40-120 mg/kg (i.p.) will have yielded a free serum concentration of 0.2 to 0.6 μM. Based upon concentration response relations determined from heterologously expressed channels, such a 2,6-DTBP concentration is anticipated to have a marked effect on gating of HCN1 channels while that of propofol is on the cusp of the effective dose. Given that both propofol (Lyashchenko et al., 2007) and 2,6-DTBP appear to act on HCN channels as gating modifiers (and not pore blockers), it is plausible to hypothesize that the sensitivity of HCN1-containing channels may be somewhat enhanced in vivo due to association with auxiliary proteins or channel modulators. Moreover, the estimates of alkylphenol potency based on inhibition of heterologously expressed channels may be somewhat underestimated due to the effect of partitioning into the lipophilic interior of Xenopus oocytes (Cacheaux et al., 2005). Together, these considerations suggest the results are consistent with the alkylphenols exerting their analgesic action via association with, and inhibition of, HCN1-containing, peripheral I_{H} channels.

TABLE 2
Relationship between total and free plasma and CSF propofol concentrations.

| | | | | | | | Dawidowicz & | |
|---|---|---|---|---|---|---|---|---|
| | Engdahl | | Dawidowicz et al., | | Dawidowicz | | Kalitynski, | |
| Reference | et al., 1998 | | 2002 | | et al., 2003A | | 2003^{d} | |
| Plasma [propofol] | μg/ml | μM^{a} | μg/ml | μM | μg/ml | μM | μg/ml | μM |
| Total | 2.2 | 12.3 | 4.5-5 | 25.2-28 | 3.3-4.2 | 18.5-23.6 | 6.1 | 34.2 |
| Free (measured) | — | — | — | — | — | — | 0.06 | 0.35 |
| Free (calculated)^{a} | 0.06 | 0.31 | 0.10 | 0.6-0.7 | 0.08-0.1 | 0.5-0.6 | 0.15 | 0.86 |
| CSF [propofol] | ng/ml | μM | ng/ml | μM | ng/ml | μM | ng/ml | μM |
| Total | 36.0 | 0.2 | 21-29 | 0.1-0.16 | 51-86 | 0.3-0.5 | 95.0 | 0.53 |
| Free | — | — | — | — | — | — | — | — |
| Total | | | | | | | ~78^{c} | 0.4 |
| Free (measured) | | | | | | | ~24^{c} | 0.1 |

^{a}Calculated free [propofol] was determined assuming that the free fraction is 2.5% of the total (Servin et al., 1988).
^{b}Data originally provided as mass/ml; molar concentrations calculated using propofol molecular weight = 178.3
^{c}Different group of subjects than used for measuring plasma and total CSF concentrations; another study from this group appears to have used the same data set (Dawidowicz et al., 2003B) as cited here, and is consequently not included.
^{d}The total plasma propofol concentration reported here is about 2-3 times greater than that which typically produces loss of response to verbal command (Smith et al., 1994; Vuyk et al., 1992; Forrest et al., 1994).

Interestingly, the CSF concentration of propofol required for anesthesia appears to be approximately one-tenth of that in plasma (Table 2 above) indicating a pharmacokinetic disequilibrium that renders such compounds weakly peripherally restricted. Examination of the extent to which 2,6-DTBP shares this property and exploitation of such a characteristic would benefit development of alkylphenols with respect to targeting peripheral sensitization.

The inventors have previously shown that propofol modifies gating of HCN channels in the closed-resting and closed-activated states with little or no action as a pore blocker (Lyashchenko et al., 2007); whether this effect is mediated via a sterically-defined site on HCN channels has not been previously addressed. A critical step in establishing that hydrophobic anesthetic-like molecules act via selective association was the observation of the “cut off” phenomena (Franks et al., 1985; Jenkins et al., 2001), in which an increase in chain length and bulk across an alkyl series leads to a loss of anesthetic efficacy of a compound despite an increase in the lipid:aqueous partition coefficient. A clear example of this is the finding that the steric arrangement of the butyl adducts of di-butylphenols completely controlled whether these isomers could modify the action of GABA_{A}-Rs (Krasowski et al., 2001).

Here, it is demonstrated that HCN1 inhibition by alkylphenols is controlled by steric factors that are a near mirror image of those that control enhancement of GABA_{A}-R gating. Thus, 2,6-DTBP retains a propofol-like selective efficacy against HCN1 channels as compared to homomeric arrangements of HCN2, 3 or 4 channels while 2,6-DSBP is a weak HCN1 antagonist. Similarly, whereas 2,4-DSBP is an effective agonist of GABA_{A}-Rs and 2,4-DTBP is ineffective, 2,4-DTBP (but not 2,4-DSBP) is an effective antagonist of HCN1. These findings indicate that selective antagonism of HCN1 channels by alkylphenols is mediated via a discrete, sterically-defined site. The observation that 2,6-DTBP is five-times more potent against HCN1 channels than is propofol indicates that selective discrimination between and within important target families (HCN versus GABA_{A}-Rs) can be achieved with minor structural changes.

The inventors have shown that the closely related anesthetic and non-anesthetic alkylphenols, propofol and 2,6-DTBP, selectively inhibit HCN1 pacemaker channels and provide selective analgesia for mechanical allodynia and mechanical and thermal hyperalgesia resulting from peripheral nerve injury. These findings establish that 2,6-DTBP (or derivatives thereof) also represent a novel class of analgesic agents that selectively target chronic neuropathic pain and a chemical lead for development of peripherally restricted HCN1-selective antagonists.

### Example 9—Determination of the HCN1 Efficacy of “Hindered”, “Leashed” or “Anchored” Propofol Derivatives

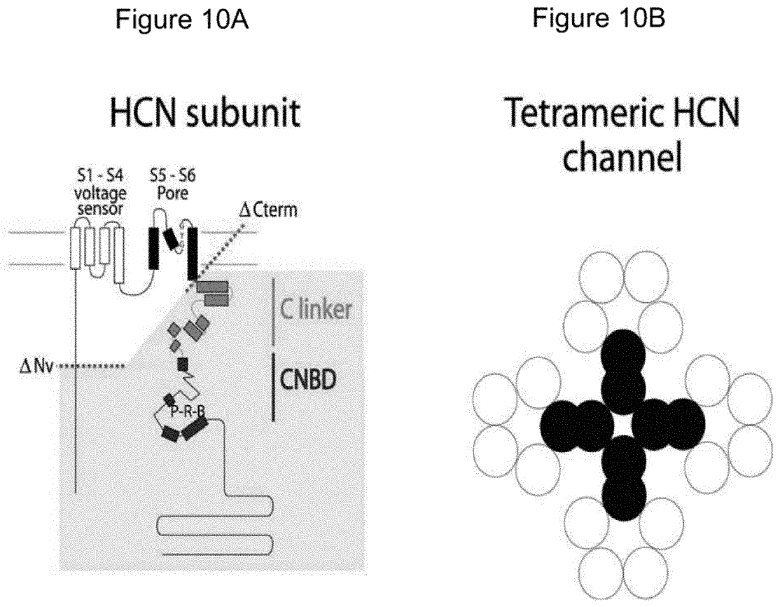

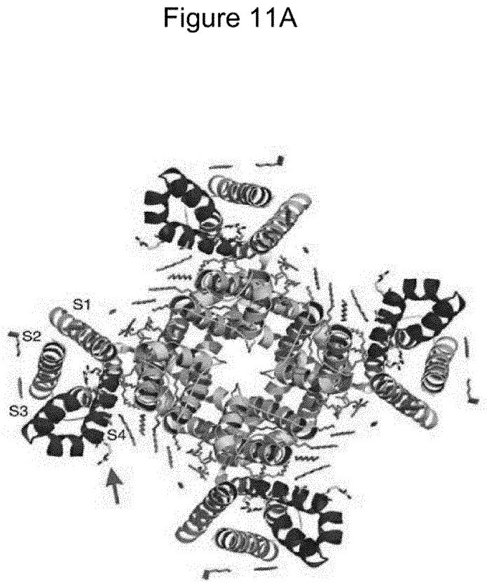

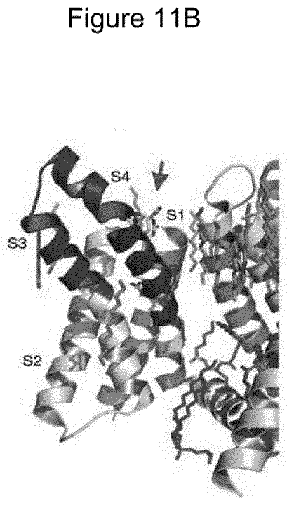

Gating of HCN channels is not only sensitive to alkylphenols but also to fatty acids such as arachidonic acid (AA) (Fogle et al., 2007). Deletion analysis reveals the membrane embedded core is sufficient for both interactions (see FIGS. 10A-10B; Lyashchenko et al., 2007; and unpublished observations). This region is highly similar between HCN1 and 4 with 283 of 308 residues being conserved (of which 269 are identical). Crystallographic analysis of Kv channels (FIGS. 11A-11B—an excellent template for interpretation of the architecture of HCN channels) show fatty acyl groups intercalate within and between the voltage sensor and pore domains (Swartz, 2008) while kinetic analysis and modeling reveals propofol acts by preferentially associating with closed HCN1 channels with little or no discrimination with regard to the activation status of the voltage sensor (Lyashchenko et al., 2007 and unpublished observations). Based on the above observations, the inventors hypothesize that the alkylphenol binding site of HCN1 lies at a superficial, inter or intra subunit, acyl accessible, pore domain interface and the drug acts by competition with native acyl groups. An alternative hypothesis would be that alkylphenols compete for water in interhelix cavities (Franks, 2008; Hemmings et al., 2005).

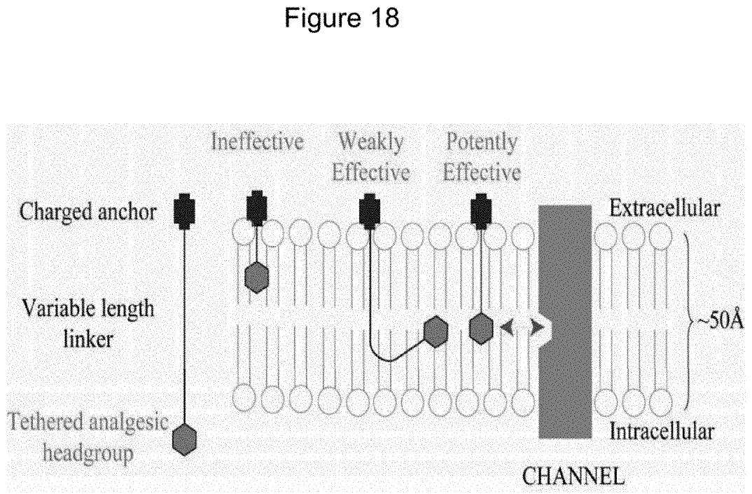

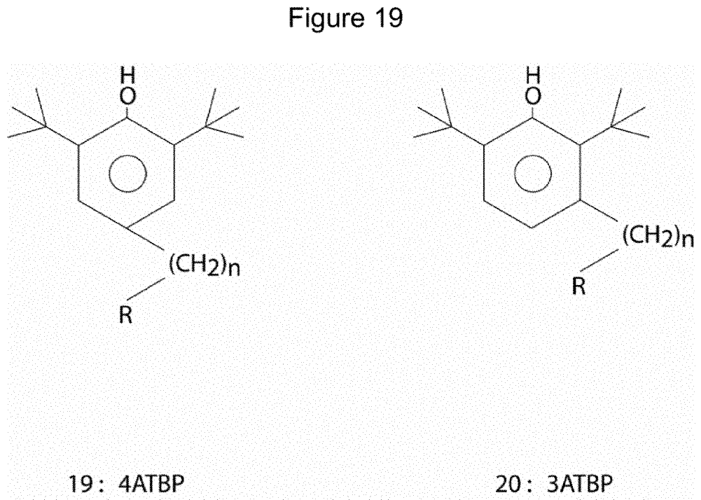

The inventors also hypothesize that propofol binds to a chemically and sterically defined site in closed HCN1 channels such that there will be a specific tolerance for the nature and position of chemical substitutions on the parental propofol molecule. This structure-activity relationship will be studied by examining a restricted series of propofol derivatives. Propofol derivatives with an additional tert-butyl or amino adduct at the 4 position are as efficacious as propofol as an HCN1 antagonist (data not shown), suggesting introduction of an alkyl-tethered “anchor” (FIG. 18) will be tolerated. These findings raise the interesting possibility that 4-substituted 2,6-DTBPs developed as COX/LOX inhibitors may be orally available HCN1 antagonists.

The efficacy and potency on HCN channels of the following propofol derivatives will be determined: “Hindered” propofols—those with aryl (pentyl or hexyl) adducts; “Leashed” propofols—those with short, medium or long primary alkyl adducts; and “Anchored” propofols—those with a quaternary ammonium anchor attached via a primary alkyl leash.

Are Bulky Aryl Substitutions of Hydrogen and Isopropyl Adducts at Positions 2 to 6 Tolerated with Respect to a Derivative's Efficacy as an HCN1 Antagonist?

To address this question, the sensitivity of the propofol inhibition of HCN1 to substitution of four specific adducts at positions 2 to 6 on the propofol ring will be probed. These adducts will be cyclopentyl or cyclohexyl groups. The rationale for the selection of these adducts is as follows:
(a) introduction of these adducts does not decrease aqueous solubility to the point where analysis is rendered impossible; and
(b) introduction of such groups eliminates coupling of propofol to GABA-A receptors and eliminates anesthetic activity in a manner that is specific for both the position of the adduct(s) on the propofol ring and geometry of the adduct. If one of these GABA-A decoupled non-anesthetic compounds were to retain an isoform selective inhibition of HCN1 it would be an attractive lead compound with respect to development of a neuropathic pain analgesic.

To examine the effect of introduction of each adduct, an abbreviated concentration response analysis will be initially conducted. Specifically, equilibrium activation curves will be collected before and after exposure of a cell to low, medium and high concentrations of the reagent. Specifically, oocytes expressing HCN1 will be placed in a glass scintillation vial containing 20 ml of the standard TEVC bath solution supplemented with 0.01% DMSO. After 20 minutes, the cell will be transferred to a recording chamber that is continuously perfused with this same solution and a complete IV protocol executed. The cell will then be transferred to a scintillation vial containing either 20 ml of the 0.01% DMSO supplemented TEVC bath solution or a vial containing the desired concentration of the propofol derivative with the DMSO concentration adjusted so that it is equal to 0.01%. After a further 20 minutes incubation, the cell will be again transferred to the recording chamber that is now perfused with either the bath solution supplemented with 0.01% DMSO or the bath solution supplemented with 0.01% DMSO plus the propofol derivative at the same concentration as was present in the incubation. A complete activation curve will again be recorded. No cell will be exposed to more than one such recording cycle. The concentrations of the propofol derivative in such an initial screen will be 10 μM, 100 μM and 500 μM. While analysis of this abbreviated concentration response series will not fully define the efficacy and potency with which a particular derivative can modify HCN1 gating, it will be sufficient to indicate whether a detailed analysis is warranted.

To limit the number of structures to be tested, only single site replacements will be tested (meaning only the adduct attached to positions 2, 3 or 4 need to be tested, because substitutions at positions 3 and 5 and at positions 2 and 6 are symmetrical in the background of the parental molecule) with the exception of those molecules already investigated with respect to activity against GABA-A and with respect to induction of anesthesia.

After identifying one or more sterically hindered propofols that can inhibit HCN1, they will be analyzed for loss of anesthetic activity unless they have been tested previously in that assay. Analysis of efficacy against HCN4 and 2 would follow a demonstration that the derivative was no longer an anesthetic.

While the chosen paradigm of analysis (determination of an activation curve pre and post an intervening incubation phase) allows for a higher throughput in the analysis, this introduces concerns with respect to drift in gating during the inter-analysis incubation periods. Such drift could, for example, be confused for a weak effect of an otherwise ineffective derivative. Although this approach was previously used satisfactorily, this remains a legitimate concern. Two steps will be taken to avoid misinterpreting the effectiveness or lack thereof of apparently ineffective or weakly effective inhibitors. First, DMSO controls will be included for each batch of oocytes. Observation of marked drift between the first and second activation curves would lead to abandonment of that batch of cells. Second, small effects will be confirmed by following gating using the “Imod” protocol. If a derivative has a modest effect on gating, this test can reveal this even in the face of some drift. The “Imod” protocol will not be used as the first line analysis, however, because quantitative interpretation of the changes in amplitude of the currents at intermediate and fully activating potentials is dependent on the slope of the gating relationship remaining constant—a feature that can only be demonstrated by construction of complete activation curves in the absence or presence of drug.

It is clearly conceivable that a substituted propofol that would be effective against HCN1 channels would appear to be ineffective if its aqueous solubility were too low. This potential problem is not anticipated for the “hindered” series of compounds, because propofol derivatives bearing multiple butyl, pentyl and hexyl adducts have been successfully analyzed by others.

As noted above, the analysis will provide an incomplete picture of the efficacy and potency of each antagonist with respect to HCN1 inhibition. Further analysis would only be warranted for those compounds that displayed the three critical phenotypes: activity against HCN1, ineffectiveness as an antagonist and ineffectiveness against HCN2 or 4. For the most promising compounds concentration response curves may be constructed and further structure-activity information regarding docking of propofol with HCN1 obtained.

Are Long Alkyl “Leash” Substitutions of Hydrogen and Isopropyl Adducts at Positions 2 to 6 Tolerated with Respect to Such Substituted Derivatives Retaining Efficacy as an HCN1 Antagonist?

To address this question, the sensitivity of the propofol inhibition of HCN1 to substitution of adducts at positions 2 to 6 on the propofol ring with short (4), medium (8) and long (12) carbon alkyl chains will be probed. The rationale for the selection of these adducts is as follows:
(a) Introduction of such a “leash” may, in its own right, generate a derivative that retains HCN1 antagonist activity while disrupting coupling to GABA-A and its ability to function as an effective anesthetic. Such a compound would be an attractive lead compound with respect to development of a neuropathic pain analgesic.
(b) Analysis of “leash” chemistry is an essential prerequisite to analysis of the effect of introducing “anchored” versions of propofol.

To examine the effect of introduction of each alkyl group modification, an abbreviated concentration response analysis will be initially conducted exactly as described above. Note that substitutions at positions 3 and 5 and at positions 2 and 6 are symmetrical in the background of the parental molecule (which has hydrogens at 3, 4 and 5 and isopropyl groups at positions 2 and 6). As the screen is only directed towards identifying whether variable length alkyl substitutions are tolerated at positions 2 to 6 in isolation (and not in combination with a second substitution in such a broken symmetry background), only derivatives at positions 2, 3 and 4 need to be screened.

It is predicted that propofol is docked into an amphiphilic pocket and that part of the molecule (most likely the hydrogens at positions 3, 4 and 5) will be in close proximity to the lipid phase. Attachment of a long alkyl “leash” at such positions will neither preclude propofol from accessing its site nor will the leash interfere with the ligand docking therein. Accordingly, within this framework, it is anticipated that substitution of an alkyl leash will not disrupt the ability of the propofol derivative from inhibiting HCN1 gating. Indeed, within the limits of aqueous solubility, longer chain substitutions may enhance the effectiveness of the propofol derivative with respect to parental propofol by leading to a higher effective concentration (and hence effective rate of drug binding) within the membranous phase.

After identifying one or more “leashed” propofols that can inhibit HCN1, they will be analyzed for loss of anesthetic activity. As outlined below, analysis of efficacy against HCN4 and 2 would follow a demonstration that the derivative was no longer an anesthetic. A propofol bearing a long alkyl leash that displayed the three critical phenotypes (activity against HCN1, ineffectiveness as an antagonist and ineffectiveness against HCN2 or 4) would also be used as a template for construction of an “anchored” propofol.

Increasing the length of the alkyl substitution will lead to a reduction in aqueous solubility. Clearly, it is expected that a primary butyl derivative will remain aqueous soluble given that such butyl derivatives have been used extensively by others. Similarly, given that the dihexyl versions of propofol introduces an extra 8 carbons compared to parental propofol, it is hypothesized that attachment of an 8 carbon “leash” will not render propofol aqueous insoluble. But what of a 12 carbon “leash”? Clearly, such an adduct may render the compound insufficiently soluble in water for it to be tested in the assays. However, it is important to note that as the primary goal of exploring the tolerance for attachment of long leashes is to be able to explore the attachment of a quaternary ammonium charged “anchor”, the demonstration that leashes as long as 8 carbons are acceptable would be sufficient validation of the concept that generation of short and long “leashed” derivatives bearing a hydrophilic “anchor” would be warranted. Such compounds would, presumably, have enhanced solubility due to the ionic anchor group.

Is Attachment of a Quaternary Ammonium “Anchor” to an Alkyl Leash Tolerated with Respect to Such Substituted Derivatives Retaining Efficacy as an HCN1 Antagonist?

To address this question, HCN1 will be probed with such molecules. The general structure of such a molecule, which comprises a charged anchor, a variable length linker, and a tethered propofol headgroup, is illustrated in FIG. 18, left side. The “tethered propofol headgroup” includes propofol, 2,6-DTBP, and 2,6-DSBP.

The attachment of a charged group will be attempted at those positions where tolerance for at least a short (4) or medium (8) alkyl “leash” attachment will be identified. If more than one position is identified, that position which shows the best HCN1 inhibitory characteristics and/or one that clearly shows tolerance for attachment of the longest “leash” will be selected.

Specifically, the efficacy with which 2,6-DTBP derivatives bearing a permanently charged anchor (attached to either the 3 or 4 position of the 2,6-DTBP headgroup) via a variable length alkyl chain tether is able to inhibit HCN1 and relieve neuropathic pain will be examined. It is predicted that the 2,6-DTBP “headgroup” will access its pharmacological site with an orientation that permits docking with, and inhibition of, HCN1 and relief of neuropathic pain. The structure of such derivatives are shown below:

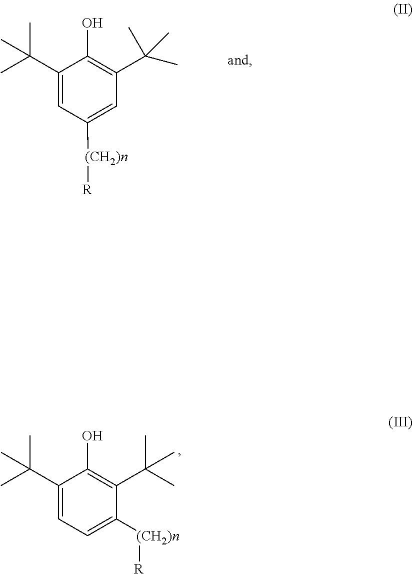

Where n=2-36, and R is a charged anchor with a permanent positive charge, but preferably will be biologically inert in its own right. Examples of such R groups are —NH₂, —N(CH₃)₃, a guanidine group, an aromatic amino group, and a quaternary ammonium group. As noted above, n may also be from 2-18, such as 4, 8, or 12.

More specifically, the efficacy with which two C₁₈-tethered quaternary ammonium derivatives of 2,6-DTBP (3C₁₈N-2,6-DTBP and 4C₁₈N-2,6-DTBP) inhibit gating of HCN1 channels will be examined. In these compounds a trimethyl ammonium “anchor” will be tethered to the 3 or 4 position of the phenolic ring via a saturated 18 carbon alkyl chain—a tether of sufficient length to allow the 2,6-DTBP “head group” to cross about half the physical distance of the bilayer. Note that a 5 substitution is redundant as it is the mirror image of addition at position 3 (FIG. 1) and that use of a saturated chain will maximize rotational flexibility of the 2,6-DTBP “headgroup”. To gain insight into the location of the alkylphenol site with respect to depth in the bilayer, the compounds will be applied to either the outer (TEVC) or inner (inside out patch clamp—IOPC) faces of the plasma membrane. It is predicted that the 2,6-DTBP “headgroup” will access its binding site with an orientation that permits docking with, and inhibition of, HCN1 but will do so from only one face of the membrane.

Can additional adducts be added to the phenolic ring and retain function? The results above revealed that such additional substituents are tolerated (indeed, it is possible that tolerance at the level of HCN1 may underlie the unexplained observation that some 2,6-DTBP derived COX2 inhibitors have analgesic activity in pain assays while other NSAIDs do not). It should also be noted that such tethered bifunctional ligands have proved to be pharmacologically potent compounds even when both headgroups are required to adopt precise docking configuration (Kramer et al., 1998; Morin et al., 2008; Mourot et al, 2007; Perez et al., 1998A; Perez et al., 1998B; Halazy et al., 1996; Halazy et al., 1999)—a constraint absent in this experiment given that the amino group will be acting simply through its preference to remain in the aqueous compartment.

If neither of these parental “tether” or “anchored” compounds are active as antagonists of HCN1 gating, one would conclude that the site is either fully buried in both closed/deactivated and open/activated arrangements of the channel (an unlikely scenario) or the tether prevents the headgroup from correctly orienting with respect to the alkylphenol binding site. Such a conclusion would suggest that less extensively modified 2,6-DTBP derivatives, such as, e.g., the water soluble, orally available derivatives generated by Pfizer shown below, may be better diagnostic compounds.

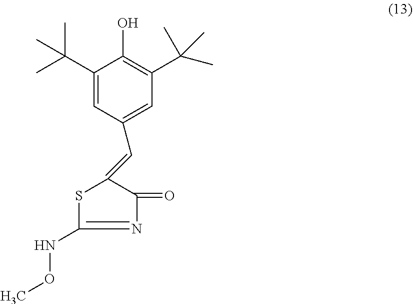 [SMILES: CONC1=NC(=O)/C(=C/c2cc(C(C)(C)C)c(O)c(C(C)(C)C)c2)S1]

C₁₈N derivatives may not have sufficient aqueous solubility to eliminate the need for a cosolvent but may not be well solvated by DHβCD (if, for example, geometry does not satisfy the DHβCD hydrophobic pocket (Brewster et al., 1994; Trapani et al., 1998; Ming-Ju et al., 2004). In this eventuality, the proposed in vitro experiments will be conducted using either larger cavity cyclodextrin derivatives (Loftsson et al., 1996) or DMSO as cosolvent. Unsaturated bonds may also be introduced into the alkyl chain, because this will enhance aqueous solubility (c.f. free fatty acids). Although solubility may be a concern, it should be noted that arachidonic acid (a 20 carbon chain bearing a single carboxylic acid group) is aqueous soluble with a critical micelle concentration of about 100 μM as determined in equivalent ionic conditions (Glick et al., 1996; Necula et al., 2003). Accordingly, it is hypothesized that even a lengthy “leash” with the relatively polar propofol “active head group” on one end combined with the polar quaternary ammonium “anchor” on the other end should have a sufficiently high solubility that “anchored” compounds bearing “leashes” greatly in excess of 8 carbons should be analyzable.

Could the quaternary ammonium anchor abolish HCN activity independently of the alkylphenol head group? This is highly unlikely given that HCN channels have little or no sensitivity to the tetraethylammonium ion itself (Ludwig et al., 1998; Ludwig et al., 1999; Solomon et al., 1993; Wollmuth et al., 1992; Pape, 1996)—indeed this is a central reason underlying the choice of this group as the charged “anchor” for these pilot studies.

It is predicted that the propofol head group of such a bifunctional molecule will be able to enter the hydrophobic phase and access its binding site on HCN1 when the “leash” is sufficiently long but there will be a cutoff phenomena such that shortening the “leash” length will eliminate the activity of the ligand when the propofol “bathysphere” can no longer penetrate sufficiently deeply into the hydrophobic phase to allow it reach its binding site.

These experiments will be conducted in a manner exactly analogous to those set forth above.

### Example 10—Determination of the Anesthetic and Cardiac Sparing Nature of HCN1-Efficacious “Hindered”, “Leashed” or “Anchored” Propofols

It is hypothesized that the determinants of propofol binding to HCN1 channels will be distinct from those that control association with propofol's primary central anesthetic target (GABA-A channels) and the HCN subunits underlying the cardiac I_{H} current (HCN2 and 4). To explore this, the efficacy and potency with which HCN1-efficacious derivatives display anesthetic activity and efficacy as antagonists of the cardiac HCN2 and 4 channels will be examined.

### Do HCN1-Efficacious “Hindered”, “Leashed” or “Anchored” Propofol Derivatives Retain Anesthetic Activity?

A complete analysis of this question would entail a major analysis of each compound. A widely adopted and technically simple assay will be used as an initial screen—the analysis of loss of righting reflex in tadpoles. As discussed extensively by others, although a surrogate for analysis in a higher animal, this assay avoids numerous issues and is generally accepted as an adequate initial surrogate screen for a compounds ability to act as an anesthetic acting via GABA-A receptors.

Do HCN1-Efficacious, Non-Anesthetic “Hindered”, “Leashed” or “Anchored” Propofol Derivatives Retain Ineffectiveness with Respect to Inhibition of the Cardiac HCN Isoforms?

It is predicted that modifying the propofol ring in the manner described above will not introduce an ability for these compounds to inhibit HCN2 or HCN4. However, to explicitly explore this issue, compounds that are found to retain an antagonist activity against HCN1 but which are found to be devoid, or largely devoid, of anesthetic activity will be tested for their ability to modify the function of HCN2 or HCN4.

To address this question, TEVC analysis of the effect of such compounds on homomerically expressed HCN2 or HCN4 will be conducted using the experimental paradigms. The only change that will be needed is that the duration of the activating voltage step will need to be lengthened to account for the slower gating of these channels. It is predicted that modification of the propofol ring will not introduce an marked efficacy of the compound against HCN2 or 4.

### Example 11—Testing of Additional Propofol Derivatives

Additional propofol derivatives shown below will be tested to determine whether they antagonize HCN1 ion channels and whether they act as a selective analgesic in animal models of neuropathic pain according to the methods set forth above.

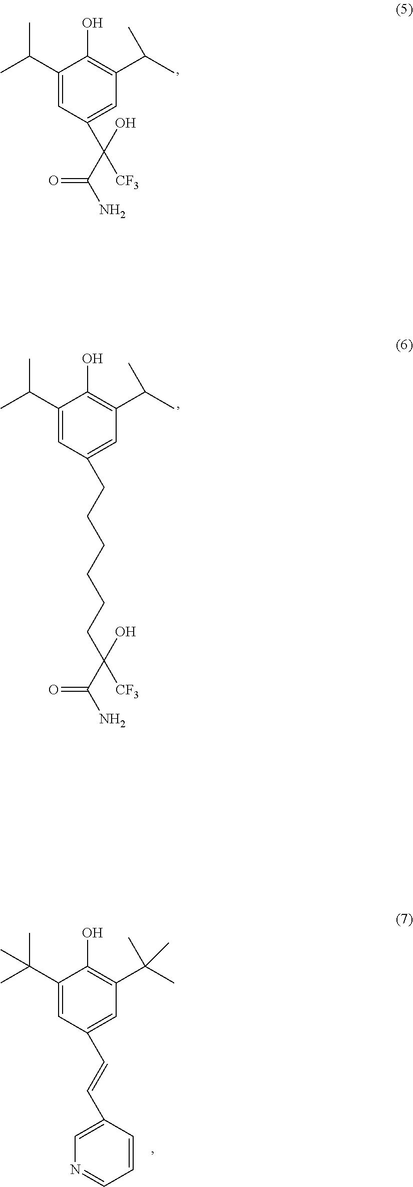

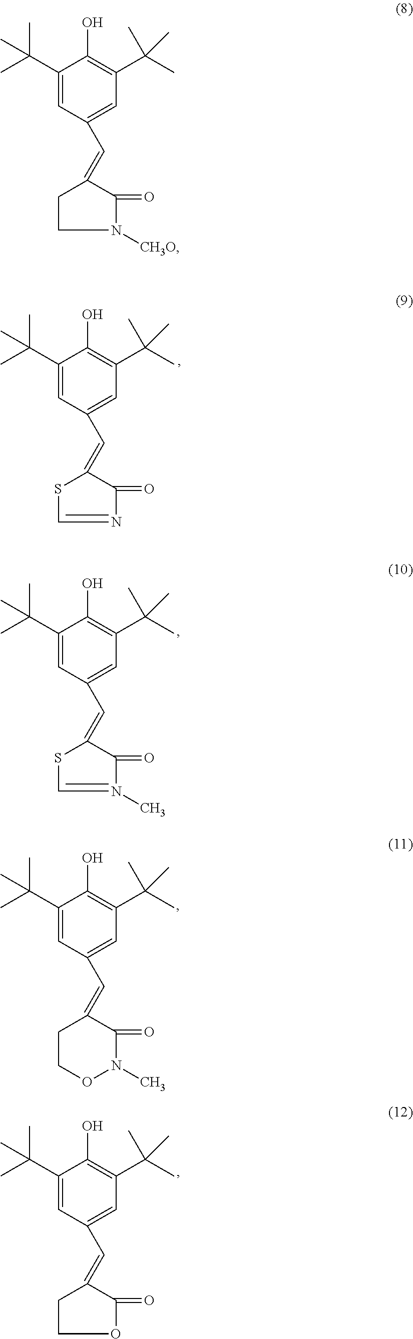

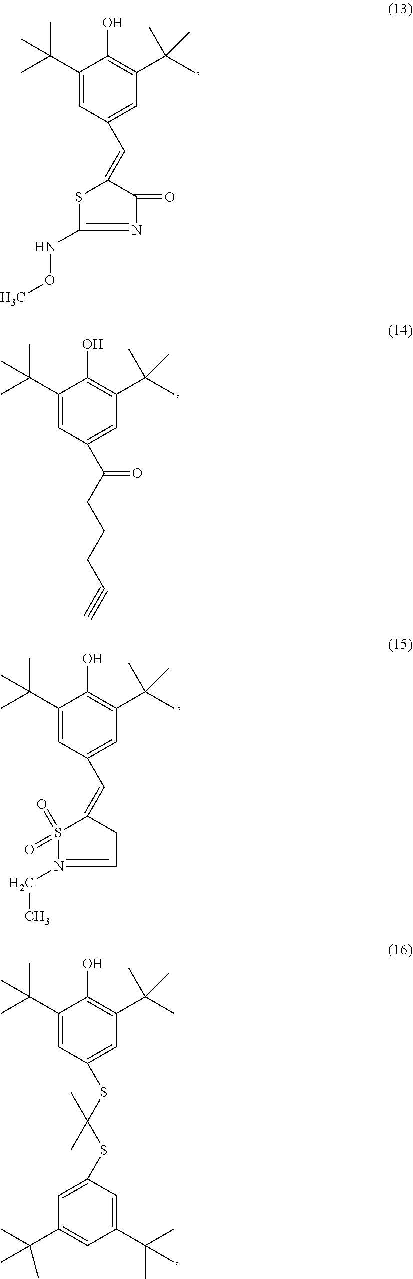

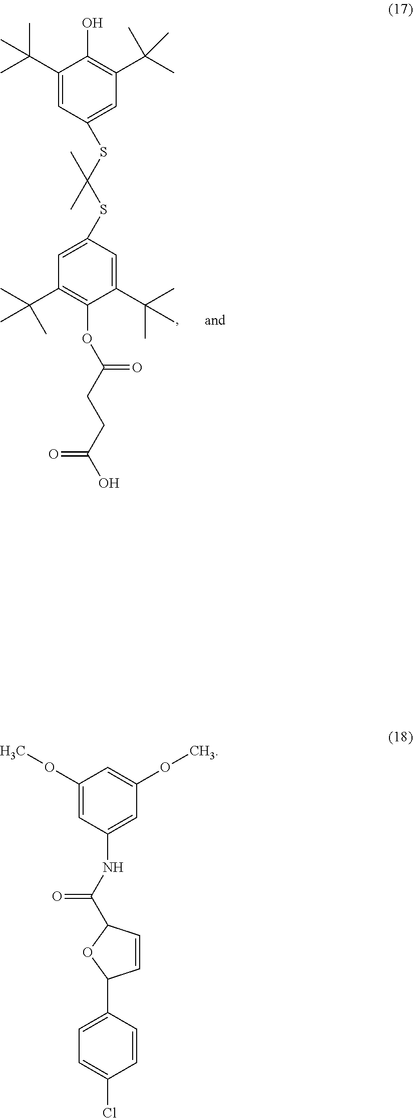

Compound 5 and Compound 6, also known as HS-245 and HS-357, respectively, may be obtained from the University of Virginia (Charlottesville, Va.) and the University of Utah (Salt Lake City, Utah). Compound 7 (Lazer et al., 1989; Lazer et al., 1990; Connor et al., 1996), also known as BI-L-93, may be obtained from Boehringer Ingelheim GmbH (Ingelheim am Rhein, Germany). Compound 8 (Katayama et al., 1987; Shirota et al., 1987; Nishibe et al, 1995; Daling et al., 1994; Shirota et al., 1989), also known as E-5110, may be obtained from Eisai Inc. (Woodcliff Lake, N.J.). Compounds 9-11 (Bendele et al., 1992), also known as LY-178002, LY-256548, and BF-389, respectively, may be obtained from Eli Lilly and Co. (Indianapolis, Ind.). Compound 12 (Hidaka et al., 1986A; Hidaka et al, 1986B; Hidaka et al., 1985; Hidaka et al., 1984), also known as KME-4, may be obtained from Kanegafuchi Chemical Industry Co. (Osaka, Japan). Compound 13 (Song et al., 1997; Mullican et al., 1993; Lesch et al., 1989; Song et al., 1999), also known as PD-138387, may be obtained from Pfizer (New York, N.Y.). Compound 14 (Doyle et al., 1993; Sietsema et al., 1993, Kaffenberger et al., 1990; Eichhold et al., 1990; Weisman et al., 1994; Janusz et al., 1998A; Janusz et al., 1998B; Janusz et al., 1998C), also known as NE-11740 or Tebufelone, may be obtained from Proctor and Gamble (Cincinnati, Ohio). Compound 15 (Yagami et al., 2005; Yagami et al., 2001; Inagaki, 2003; Inagaki et al., 2003; Inagaki et al., 2000; Oda et al., 2008), also known as S-2474, may be obtained from Shionogi & Co. (Osaka, Japan). Compound 16 (Yamashita et al., 2009; Yamamoto, 2008; Stocker, 2009) is available under the trade names Biphenabid, Bisbid, Bisphenabid, Lesterol, Lorelco, Lursell, Lurselle, Panavir, and Sinlestal from Dow Chemical Co. (Indianapolis, Ind.). Compound 17, also known as AGI-1067, may be obtained from Astra-Zeneca Pharmaceuticals LP (Willimgton, Del.). Finally, Compound 18 (Jarvis et al., 2007), also known as A-803467, may be obtained from Abbott Laboratories (Abbott Park, Ill.)/Icagen Inc. (Durham, N.C.).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### CITED DOCUMENTS

Patents, patent applications, publications, procedures, and the like are cited throughout this application and are cited below, the disclosures of which are incorporated herein by reference in their entireties.
Abbas, S. Y., S. W. Ying, and P. A. Goldstein, Compartmental distribution of hyperpolarization-activated cyclic-nucleotide-gated channel 2 and hyperpolarization-activated cyclic-nucleotide-gated channel 4 in thalamic reticular and thalamocortical relay neurons. Neuroscience, 2006. 141(4): p. 1811-25.
Accili, E. A., et al., From funny current to HCN channels: 20 years of excitation. News Physiol Sci, 2002. 17: p. 32-7.
Ahrens, J., et al., 2,6 di-tert-butylphenol, a nonanesthetic propofol analog, modulates a1b glycine receptor function in a manner distinct from propofol. Anesth Analg, 2004. 99(1): p. 91-6.
Ahrens, J., et al., The non-anaesthetic propofol analogue 2,6-di-tert-butylphenol fails to modulate GABA_{A} receptor function. Pharmacology, 2009. 83(2): p. 95-8.
Altomare, C., et al., Heteromeric HCN1-HCN4 channels: a comparison with native pacemaker channels from the rabbit sinoatrial node. J Physiol, 2003. 549(Pt 2): p. 347-59.
Amaya, F., et al., The voltage-gated sodium channel Na(v)1.9 is an effector of peripheral inflammatory pain hypersensitivity. J Neurosci, 2006. 26(50): p. 12852-60.
Azene, E. M., et al., Non-equilibrium behavior of HCN channels: insights into the role of HCN channels in native and engineered pacemakers. Cardiovasc Res, 2005. 67(2): p. 263-73.
Azene, E. M., et al., Pore-to-gate coupling of HCN channels revealed by a pore variant that contributes to gating but not permeation. Biochem Biophys Res Commun, 2005. 327(4): p. 1131-42.
Baker, M. T. and M. Naguib, Propofol: the challenges of formulation. Anesthesiology, 2005. 103(4): p. 860-76.
Bal, T. and D. A. McCormick, Synchronized oscillations in the inferior olive are controlled by the hyperpolarization-activated cation current I(h). J Neurophysiol, 1997. 77(6): p. 3145-56.
Bali, M. and M. H. Akabas, Defining the propofol binding site location on the GABAA receptor. Mol Pharmacol, 2004. 65(1): p. 68-76.
Barbuti, A., M. Baruscotti, and D. DiFrancesco, The pacemaker current: from basics to the clinics. J Cardiovasc Electrophysiol, 2007. 18(3): p. 342-7.
Baruscotti, M., A. Barbuti, and A. Bucchi, The cardiac pacemaker current. J Mol Cell Cardiol, 2010. 48: p. 55-64.
Beaumont, V. and R. S. Zucker, Enhancement of synaptic transmission by cyclic AMP modulation of presynaptic Ih channels. Nat Neurosci, 2000. 3(2): p. 133-41.
Becker, C. M., et al., Isoform-selective deficit of glycine receptors in the mouse mutant spastic. Neuron, 1992. 8(2): p. 283-9.
Bedell, S. A. and A. E. Martell, Oxidation of 2,6-di-tert-butylphenol by molecular oxygen. Catalysis by tetrakis(bipyridyl)(mu.-peroxo)(mu.-hydroxo)dicobalt(III). Inorganic Chemistry, 1983. 22(2): p. 364-367.
Beggs, S. and M. Salter, W., Neuropathic pain: symptoms, models, and mechanisms. Drug Development Research, 2006. 67(4): p. 289-301.
Belelli, D., et al., The interaction of general anaesthetics and neurosteroids with GABA_{A} and glycine receptors. Neurochem Int, 1999. 34(5): p. 447-52.
Bell, D. C., Yao, H., Saenger, R. C., Riley, J. H., and Siegelbaum, S. A., Changes in Local S4 Environment Provide a Voltage-sensing Mechanism for Mammalian Hyperpolarization-activated HCN Channels., 2003. J Gen Physiol. 123, 5-20.
Bendele, A. M., et al., Anti-inflammatory activity of BF389, a Di-T-butylphenol, in animal models of arthritis. J Pharmacol Exp Ther, 1992. 260(1): p. 300-5.
Bender, R. A., et al., Localization of HCN1 channels to presynaptic compartments: novel plasticity that may contribute to hippocampal maturation. J Neurosci, 2007. 27(17): p. 4697-706.
Bennett, G. J. and Y. K. Xie, A peripheral mononeuropathy in rat that produces disorders of pain sensation like those seen in man. Pain, 1988. 33(1): p. 87-107.
Berger, T., M. E. Larkum, and H. R. Luscher, High I(h) channel density in the distal apical dendrite of layer V pyramidal cells increases bidirectional attenuation of EPSPs. J Neurophysiol, 2001. 85(2): p. 85
Berger, T., W. Senn, and H. R. Luscher, Hyperpolarization-Activated Current Ih Disconnects Somatic and Dendritic Spike Initiation Zones in Layer V Pyramidal Neurons. J Neurophysiol, 2003. 90(4): p. 2428-2437
Biel, M., A. Schneider, and C. Wahl, Cardiac HCN channels: structure, function, and modulation. Trends Cardiovasc Med, 2002. 12(5): p. 206-12.
Biel, M., Cyclic Nucleotide-regulated Cation Channels. J Biol Chem, 2009. 284(14): p. 9017-21.
Biel, M., et al., Hyperpolarization-activated cation channels: a multi-gene family. Rev Physiol Biochem Pharmacol, 1999. 136: p. 165-81.
Biel, M., et al., Hyperpolarization-activated cation channels: from genes to function. Physiol Rev, 2009. 89(3): p. 847-85.
Bingham, S., et al., The cyclooxygenase-2 inhibitor GW406381X [2-(4-ethoxyphenyl)-3-[4-(methylsulfonyl)phenyl]-pyrazolo[1,5-b]pyridazine] is effective in animal models of neuropathic pain and central sensitization. J Pharmacol Exp Ther, 2005. 312(3): p. 1161-9.
Brewster, M. E., et al., Solubilization and electrochemical stabilization of substituted phenols through the use of 2-hydroxypropyl-β-cyclodextrin Supramolecular Chemistry, 1994. 4(1): p. 69-76.
Brewster, M. E., K. S. Estes, and N. Bodor, An intravenous toxicity study of 2-hydroxypropyl-β-cyclodextrin, a useful drug solubilizer, in rats and monkeys. Int J Pharm, 1990. 59: p. 231-243.
Broom, D. C., et al., Cyclooxygenase 2 expression in the spared nerve injury model of neuropathic pain. Neuroscience, 2004. 124(4): p. 891-900.
Brown, S. M., A. E. Dubin, and S. R. Chaplan, The role of pacemaker currents in neuropathic pain. Pain Pract, 2004. 4(3): p. 182-93.
Bruening-Wright, A. and H. P. Larsson, Slow conformational changes of the voltage sensor during the mode shift in hyperpolarization-activated cyclic-nucleotide-gated channels. J Neurosci, 2007. 27(2): p. 27
Bruening-Wright, A., F. Elinder, and H. P. Larsson, Kinetic relationship between the voltage sensor and the activation gate in spHCN channels. J Gen Physiol, 2007. 130(1): p. 71-81.
Bucchi, A., et al., Heart rate reduction via selective ‘funny’ channel blockers. Curr Opin Pharmacol, 2007. 7(2): p. 208-13.
Bucchi, A., et al., Properties of ivabradine-induced block of HCN1 and HCN4 pacemaker channels. J Physiol, 2006. 572(Pt 2): p. 335-46.
Bucchi, A., M. Baruscotti, and D. DiFrancesco, Current-dependent block of rabbit sino-atrial node I(f) channels by ivabradine. J Gen Physiol, 2002. 120(1): p. 1-13.
Cacheaux, L. P., et al., Impairment of hyperpolarization-activated, cyclic nucleotide-gated channel function by the intravenous general anesthetic propofol. J Pharmacol Exp Ther, 2005. 315(2): p. 517-25.
Campbell, J. N. and R. A. Meyer, Mechanisms of neuropathic pain. Neuron, 2006. 52(1): p. 77-92.
Cashman, J. N., The mechanisms of action of NSAIDs in analgesia. Drugs, 1996. 52 Suppl 5: p. 13-23.
Chan, C. S., et al., HCN2 and HCN1 channels govern the regularity of autonomous pacemaking and synaptic resetting in globus pallidus neurons. J Neurosci, 2004. 24(44): p. 9921-32.
Chang, L., et al., Inhibition of fatty acid amide hydrolase produces analgesia by multiple mechanisms. Br J Pharmacol, 2006. 148(1): p. 102-13.
Chaplan, S. R., et al., Neuronal hyperpolarization-activated pacemaker channels drive neuropathic pain. J Neurosci, 2003. 23(4): p. 1169-78.
Chen, S., et al., Voltage sensor movement and cAMP binding allosterically regulate an inherently voltage-independent closed-open transition in HCN channels. J Gen Physiol, 2007. 129(2): p. 175-88.
Chen, S., J. Wang, and S. A. Siegelbaum, Properties of hyperpolarization-activated pacemaker current defined by coassembly of HCN1 and HCN2 subunits and basal modulation by cyclic nucleotide. J Gen Physiol, 117, 491-504
Chen, X., et al., HCN subunit-specific and cAMP-modulated effects of anesthetics on neuronal pacemaker currents. J Neurosci, 2005. 25(24): p. 5803-14.
Chen, X., et al., Subunit-Specific Effects of Isoflurane on Neuronal Ih in HCN1 Knockout Mice. J Neurophysiol, 2009. 101(1): p. 129-40.
Chen, X., S. Shu, and D. A. Bayliss, HCN1 channel subunits are a molecular substrate for hypnotic actions of ketamine. J Neurosci, 2009. 29(3): p. 600-9.
Chen, X., S. Shu, and D. A. Bayliss, Suppression of Iₕ contributes to propofol-induced inhibition of mouse cortical pyramidal neurons. J Neurophysiol, 2005. 94(6): p. 3872-83.
Cheng, L., et al., Molecular Mapping of the Binding Site for a Blocker of HCN2 Pacemaker Channels. J Pharmacol Exp Ther, 2007.
Cheng, V. Y., et al., Alpha5GABAA receptors mediate the amnestic but not sedative-hypnotic effects of the general anesthetic etomidate. J Neurosci, 2006. 26(14): p. 3713-20.
Cho, H. J., et al., Hyperpolarization-activated cyclic-nucleotide gated 4 (HCN4) protein is expressed in a subset of rat dorsal root and trigeminal ganglion neurons. Cell Tissue Res, 2009. 338(2): p. 171-7.
Cohen, I. S. and R. B. Robinson, Pacemaker current and automatic rhythms: toward a molecular understanding. Handb Exp Pharmacol, 2006(171): p. 41-71.
Colquhoun, D., Binding, gating, affinity and efficacy: the interpretation of structure-activity relationships for agonists and of the effects of mutating receptors. Br J Pharmacol, 1998. 125(5): p. 924-47.
Connor, D. T. and D. H. Boschelli, Dual inhibitors of 5-lipooxygenase and cyclooxygenase. Biological Inhibitors (Studies in Medicinal Chemistry), ed. I. M. Choudhary. Vol. 2. 1996: Harwood Academic Publishers. 494.
Costigan, M., J. Scholz, and C. J. Woolf, Neuropathic pain: a maladaptive response of the nervous system to damage. Annu Rev Neurosci, 2009. 32: p. 1-32.
Craven, K. B. and W. N. Zagotta, CNG and HCN Channels: Two Peas, One Pod. Annu Rev Physiol, 2006. 68:375-401.
Cummins, T. R., P. L. Sheets, and S. G. Waxman, The roles of sodium channels in nociception: Implications for mechanisms of pain. Pain, 2007. 131(3): p. 243-57.
Cuttle, M. F., et al., Modulation of a presynaptic hyperpolarization-activated cationic current (I(h)) at an excitatory synaptic terminal in the rat auditory brainstem. J Physiol, 2001. 534(Pt 3): p. 733-44
Daling, Z., et al., Effect of E-5110, a novel non-steroidal anti-inflammatory drug, on trimethadione metabolism as an indicator of hepatic drug-oxidizing capacity in beagle dog. Xenobiotica, 1994. 24(3): p
Dalle, C. and Eisenach, J. C., Peripheral block of the hyperpolarization-activated cation current (Iₕ) reduces mechanical allodynia in animal models of postoperative and neuropathic pain, 2005 Reg Anesth Pain Med, Vol. 30, No. 3.
Davis, M. P., et al., Controversies in pharmacotherapy of pain management. Lancet Oncol, 2005. 6(9): p. 696-704.
Dawidowicz, A. L. and R. Kalitynski, HPLC investigation of free and bound propofol in human plasma and cerebrospinal fluid. Biomed Chromatogr, 2003. 17(7): p. 447-52.
Dawidowicz, A. L., et al., Cerebrospinal fluid and blood propofol concentration during total intravenous anaesthesia for neurosurgery. Br J Anaesth, 2003A. 90(1): p. 84-6.
Dawidowicz, A. L., et al., Changes of propofol concentration in cerebrospinal fluid during continuous infusion. Anesth Analg, 2002. 95(5): p. 1282-4.
Dawidowicz, A. L., R. Kalitynski, and A. Fijalkowska, Free and bound propofol concentrations in human cerebrospinal fluid. Br J Clin Pharmacol, 2003B. 56(5): p. 545-50.
Decosterd, I. and C. J. Woolf, Spared nerve injury: an animal model of persistent peripheral neuropathic pain. Pain, 2000. 87(2): p. 149-58.
Delmas, P., SnapShot: Ion Channels and Pain. Cell, 2008. 134(2): p. 366-366.e1.
Deschenes, M., J. P. Roy, and M. Steriade, Thalamic bursting mechanism: an inward slow current revealed by membrane hyperpolarization. Brain Res, 1982. 239(1): p. 289-293.
Desjardins, A. E., et al., The influences of Ih on temporal summation in hippocampal CA1 pyramidal neurons: a modeling study. J Comput Neurosci, 2003. 15(2): p. 131-42.
Destexhe, A. and A. Babloyantz, A model of the inward current Ih and its possible role in thalamocortical oscillations. NeuroReport, 1993. 4(2): p. 223-226.
Destexhe, A., A. Babloyantz, and T. J. Sejnowski, Ionic mechanisms for intrinsic slow oscillations in thalamic relay neurons. Biophys J, 1993. 65(4): p. 1538-1552.
Destexhe, A., D. Contreras, and M. Steriade, Mechanisms underlying the synchronizing action of corticothalamic feedback through inhibition of thalamic relay cells. J Neurophysiol, 1998. 79(2): p. 999-1016.
Dhaka, A., V. Viswanath, and A. Patapoutian, Trp ion channels and temperature sensation. Annu Rev Neurosci, 2006. 29: p. 135-61.
Dib-Hajj, S. D., et al., From genes to pain: Na v 1.7 and human pain disorders. Trends Neurosci, 2007. 30(11): p. 555-63.
Dib-Hajj, S. D., J. A. Black, and S. G. Waxman, Voltage-gated sodium channels: therapeutic targets for pain. Pain Med, 2009. 10(7): p. 1260-9.
Dickson, C. T., et al., Properties and role of 1(h) in the pacing of subthreshold oscillations in entorhinal cortex layer II neurons. J Neurophysiol, 2000. 83(5): p. 2562-79.
Dieleman, J. P., et al., Incidence rates and treatment of neuropathic pain conditions in the general population. Pain, 2008. 137(3): p. 681-8.
DiFrancesco, D., Cardiac pacemaker I(f) current and its inhibition by heart rate-reducing agents. Curr Med Res Opin, 2005. 21(7): p. 1115-22.
DiFrancesco, D., Funny channels in the control of cardiac rhythm and mode of action of selective blockers. Pharmacol Res, 2006. 53(5): p. 399-406.
DiFrancesco, D., Pacemaker mechanisms in cardiac tissue. [Review] [114 refs]. Annual Review of Physiology, 1993. 55: p. 455-472.
DiFrancesco, D., The cardiac hyperpolarizing-activated current, if. Origins and developments. [Review][85 refs]. Progress in Biophysics & Molecular Biology, 1985. 46: p. 163-183.
DiFrancesco, D., The onset and autonomic regulation of cardiac pacemaker activity: relevance of the f current. [Review] [68 refs]. Cardiovascular Research, 1995. 29: p. 449-456.
Djouhri, L. and S. N. Lawson, Abeta-fiber nociceptive primary afferent neurons: a review of incidence and properties in relation to other afferent A-fiber neurons in mammals. Brain Res Brain Res Rev, 2004. 4
Djouhri, L., et al., Spontaneous pain, both neuropathic and inflammatory, is related to frequency of spontaneous firing in intact C-fiber nociceptors. J Neurosci, 2006. 26(4): p. 1281-92.
Doan, T. N., et al., Differential Distribution and Function of Hyperpolarization-Activated Channels in Sensory Neurons and Mechanosensitive Fibers. J. Neurosci., 2004. 24(13): p. 3335-3343.
Doyle, M. J., et al., Comparison of tebufelone distribution in rat blood plasma and inflamed and noninflamed tissues following peroral and intravenous administration. J Pharm Sci, 1993. 82(8): p. 847-50.
Dray, A., Neuropathic pain: emerging treatments. Br J Anaesth, 2008. 101(1): p. 48-58.
Dworkin, R. H., et al., Advances in neuropathic pain: diagnosis, mechanisms, and treatment recommendations. Arch Neurol, 2003. 60(11): p. 1524-34.
Egan, T. D., et al., The pharmacokinetics and pharmacodynamics of propofol in a modified cyclodextrin formulation (Captisol) versus propofol in a lipid formulation (Diprivan): an electroencephalographic and hemodynamic study in a porcine model. Anesth Analg, 2003. 97(1): p. 72-9, table of contents.
Eichhold, T. H. and M. J. Doyle, Determination of tebufelone, a new anti-inflammatory drug, in plasma and tissue using capillary gas chromatography/stable isotope dilution mass spectrometry. Biomed Environ Mass Spectrom, 1990. 19(4): p. 230-4.
Elinder, F., et al., Mode shifts in the voltage gating of the mouse and human HCN2 and HCN4 channels. J Physiol, 2006. 575(Pt 2): p. 417-31.
Engdahl, O., et al., Cerebrospinal fluid concentrations of propofol during anaesthesia in humans. Br J Anaesth, 1998. 81(6): p. 957-9.
Fan, S. Z., et al., Propofol concentration monitoring in plasma or whole blood by gas chromatography and high-performance liquid chromatography. Anesth Analg, 1995. 81(1): p. 175-8.
Fogle, K. J., et al., HCN pacemaker channel activation is controlled by acidic lipids downstream of diacylglycerol kinase and phospholipase A2. J Neurosci, 2007. 27(11): p. 2802-14.
Folgering, J. H. A., et al., Molecular basis of the mammalian pressure-sensitive ion channels: Focus on vascular mechanotransduction. Progress in Biophysics and Molecular Biology. 97(2-3): p. 180-195.
Forrest, F. C., et al., Propofol infusion and the suppression of consciousness: the EEG and dose requirements. Br J Anaesth, 1994. 72(1): p. 35-41.
Fowler, C. J., et al., The case for the development of novel analgesic agents targeting both fatty acid amide hydrolase and either cyclooxygenase or TRPV1. Br J Pharmacol, 2009. 156(3): p. 412-9.
Franks, N. P. and W. R. Lieb, Mapping of general anaesthetic target sites provides a molecular basis for cutoff effects. Nature, 1985. 316(6026): p. 349-51.
Franks, N. P., General anaesthesia: from molecular targets to neuronal pathways of sleep and arousal. Nat Rev Neurosci, 2008. 9(5): p. 370-86.
Franks, N. P., Molecular targets underlying general anaesthesia. Br J Pharmacol, 2006. 147 Suppl 1: p. S72-81.
Freynhagen, R. and M. I. Bennett, Diagnosis and management of neuropathic pain. BMJ, 2009. 339: p. b3002.
Fujiyama, H., et al., Liquid-Phase Oxidation of 2,6-Di-tert-butylphenol with Cu-Impregnated MCM-41 Catalysts in the Presence of Alkali Metals. Journal of Catalysis, 1999. 188(2): p. 417-425.
Gauss, R. and R. Seifert, Pacemaker oscillations in heart and brain: a key role for hyperpolarization activated cation channels. Chronobiol Int, 2000. 17(4): p. 453-69.
Gill, C., et al., Characterization of the human HCN1 channel and its inhibition by capsazepine. Br J Pharmacol, 2004. 143(3): p. 411-21.
Gilron, I. and T. J. Coderre, Emerging drugs in neuropathic pain. Expert Opin Emerg Drugs, 2007. 12(1): p. 113-26.
Glick, J., Santoyo, G., and Casey, P. J. (1996) J Biol Chem 271, 2949-2954.
Goulding, E. H., et al., Role of H5 domain in determining pore diameter and ion permeation through cyclic nucleotide-gated channels. Nature, 1993. 364: p. 61-64.
Green, B., G., Temperature perception and nociception. Journal of Neurobiology, 2004. 61(1): p. 13-29.
Gulledge, A. T., B. M. Kampa, and G. J. Stuart, Synaptic integration in dendritic trees. J Neurobiol, 2005. 64(1): p. 75-90.
Haeseler, G. and M. Leuwer, High-affinity block of voltage-operated rat IIA neuronal sodium channels by 2,6 di-tert-butylphenol, a propofol analogue. Eur J Anaesthesiol, 2003. 20(3): p. 220-4.
Haeseler, G. and M. Leuwer, High-affinity block of voltage-operated rat IIA neuronal sodium channels by 2,6 di-tert-butylphenol, a propofol analogue. Eur J Anaesthesiol, 2003. 20(3): p. 220-4.
Hains, B. C., et al., Upregulation of sodium channel Nav1.3 and functional involvement in neuronal hyperexcitability associated with central neuropathic pain after spinal cord injury. J Neurosci, 2003. 23(26): p. 8881-92.
Halazy, S., et al., Serotonin dimers: application of the bivalent ligand approach to the design of new potent and selective 5-HT(1B/1D) agonists. J Med Chem, 1996. 39(25): p. 4920-7.
Halazy, S., G-protein coupled receptors bivalent ligands and drug design. Expert Opinion on Therapeutic Patents, 1999. 9: p. 431-446.
Hassanein, M., A. Selim, and H. El-Hamshary, Oxidation of 2,6-di-tert-butylphenol by molecular oxygen catalyzed by tetrasodium phthalocyaninatocobalt(II)tetrasulfonate bound to a polymer colloid. Macromolecular Chemistry and Physics, 1994. 195: p. 3845-3854.
Hausser, M., N. Spruston, and G. J. Stuart, Diversity and dynamics of dendritic signaling. Science, 2000. 290(5492): p. 739-44.
Heinricher, M. M., et al., Descending control of nociception: Specificity, recruitment and plasticity. Brain Res Rev, 2009. 60(1): p. 214-25.
Heinricher, M. M., et al., Descending control of nociception: Specificity, recruitment and plasticity. Brain Res Rev, 2009. 60(1): p. 214-25.
Hemmings, H. C., Jr., et al., Emerging molecular mechanisms of general anesthetic action. Trends Pharmacol Sci, 2005. 26(10): p. 503-10.
Herrero, J. F., et al., Antinociception and the new COX inhibitors: research approaches and clinical perspectives. CNS Drug Rev, 2003. 9(3): p. 227-52.
Hidaka, T., et al., Analgesic and anti-inflammatory activities in rats of alpha-(3,5-di-t-butyl-4-hydroxybenzylidene)-gamma-butyrolactone (KME-4), and its intestinal damage. J Pharm Pharmacol, 1986A. 38(10): p. 748-53.
Hidaka, T., et al., Inhibition of polymorphonuclear leukocyte 5-lipoxygenase and platelet cyclooxygenase by alpha-(3,5-di-tert-butyl-4-hydroxybenzylidene)-gamma-butyrolactone (KME-4), a new antiinflammatory drug. Jpn J Pharmacol, 1985. 38(3): p. 267-72.
Hidaka, T., et al., Pharmacological properties of a new anti-inflammatory compound, alpha-(3,5-di-tertbutyl-4-hydroxybenzylidene)-gamma-butyrolactone (KME-4), and its inhibitory effects on prostaglandin synthetase and 5-lipoxygenase. Jpn J Pharmacol, 1984. 36(1): p. 77-85.
Hidaka, T., et al., The effect of alpha-(3,5-di-t-butyl-4-hydroxybenzylidene)-gamma-butyrolactone (KME-4), a new anti-inflammatory drug, on leucocyte migration in rat carrageenan pleurisy. J Pharm Pharmacol, 1986B. 38(3): p. 244-7.
Honore, E., The neuronal background K2P channels: focus on TREK1. Nat Rev Neurosci, 2007. 8(4): p. 251-61.
Hu, J., et al., Evidence for a protein tether involved in somatic touch. Embo J. 29(4): p. 855-867.
Hu, J., N. Milenkovic, and G. R. Lewin, The high threshold mechanotransducer: a status report. Pain, 2006. 120(1-2): p. 3-7.
Hughes, S. W., D. W. Cope, and V. Crunelli, Dynamic clamp study of Ih modulation of burst firing and delta oscillations in thalamocortical neurons in vitro [In Process Citation]. Neuroscience, 1998. 87(3): p.
Hutcheon, B., R. M. Miura, and E. Puil, Subthreshold membrane resonance in neocortical neurons. J Neurophysiol, 1996. 76(2): p. 683-697.
Inagaki, M., [Studies on the new antiarthritic drug candidate S-2474]. Yakugaku Zasshi, 2003. 123(5): p. 323-30.
Inagaki, M., et al., Novel antiarthritic agents with 1,2-isothiazolidine-1,1-dioxide (gamma-sultam) skeleton: cytokine suppressive dual inhibitors of cyclooxygenase-2 and 5-lipoxygenase. J Med Chem, 2000.
Inagaki, M., et al., Synthesis and activities of oxidative metabolites of the antiarthritic drug candidate S-2474. Bioorg Med Chem, 2003. 11(11): p. 2415-9.
Ishii, T. M., et al., Molecular characterization of the hyperpolarization-activated cation channel in rabbit heart sinoatrial node. J Biol Chem, 1999. 274(18): p. 12835-9.
Ishii, T. M., et al., Peripheral N- and C-terminal domains determine deactivation kinetics of HCN channels. Biochem Biophys Res Commun, 2007. 359(3): p. 592-8.
Ishii, T. M., M. Takano, and H. Ohmori, Determinants of activation kinetics in mammalian hyperpolarization-activated cation channels. J Physiol, 2001. 537(Pt 1): p. 93-100.
James, R. and J. B. Glen, Synthesis, biological evaluation, and preliminary structure-activity considerations of a series of alkylphenols as intravenous anesthetic agents. J Med Chem, 1980. 23(12): p. 1350-7.
Janusz, J. M., et al., New cyclooxygenase-2/5-lipoxygenase inhibitors. 3. 7-tert-butyl-2, 3-dihydro-3,3-dimethylbenzofuran derivatives as gastrointestinal safe antiinflammatory and analgesic agents: variations at the 5 position. J Med Chem, 1998A. 41(18): p. 3515-29.
Janusz, J. M., et al., New cyclooxygenase-2/5-lipoxygenase inhibitors. 2. 7-tert-butyl-2,3-dihydro-3,3-dimethylbenzofuran derivatives as gastrointestinal safe antiinflammatory and analgesic agents: variations of the dihydrobenzofuran ring. J Med Chem, 1998B. 41(7): p. 1124-37.
Janusz, J. M., et al., New cyclooxygenase-2/5-lipoxygenase inhibitors. 1. 7-tert-butyl-2,3-dihydro-3,3-dimethylbenzofuran derivatives as gastrointestinal safe antiinflammatory and analgesic agents: discovery and variation of the 5-keto substituent. J Med Chem, 1998C. 41(7): p. 1112-23.
Jarvis, M. F., et al., A-803467, a potent and selective Nav1.8 sodium channel blocker, attenuates neuropathic and inflammatory pain in the rat. Proceedings of the National Academy of Sciences, 2007. 104(20): p. 8520-8525.
Jenkins, A., et al., Evidence for a common binding cavity for three general anesthetics within the GABA_{A} receptor. J Neurosci, 2001. 21(6): p. RC136.
Jensen, T. S., C. S. Madsen, and N. B. Finnerup, Pharmacology and treatment of neuropathic pains. Curr Opin Neurol, 2009. 22(5): p. 467-74.
Jiang, Y. Q., et al., Axonal accumulation of hyperpolarization-activated cyclic nucleotide-gated cation channels contributes to mechanical allodynia after peripheral nerve injury in rat. Pain, 2008. 137(3): p. 495-506.
Jiang, Y. Q., et al., Characteristics of HCN channels and their participation in neuropathic pain. Neurochem Res, 2008. 33(10): p. 1979-89.
Johnston, D. and R. Narayanan, Active dendrites: colorful wings of the mysterious butterflies. Trends Neurosci, 2008. 31(6): p. 309-16.
Jones, P. J., et al., Hydroxyamide analogs of propofol exhibit state-dependent block of sodium channels in hippocampal neurons: implications for anticonvulsant activity. J Pharmacol Exp Ther, 2007. 320(2):
Julius, D. and A. I. Basbaum, Molecular mechanisms of nociception. Nature, 2001. 413(6852): p. 203-10.
Jurd, R., et al., General anesthetic actions in vivo strongly attenuated by a point mutation in the GABA_{A} receptor b3 subunit. FASEB J, 2003. 17(2): p. 250-2.
Kaffenberger, R. M., T. H. Eichhold, and M. J. Doyle, Determination of tebufelone (a new antiinflammatory drug) strength and stability in bulk drug, dosage formulations and feed admixtures by reversed-phase high-performance liquid chromatography. J Chromatogr, 1990. 505(2): p. 349-56.
Kajander, K. C. and G. J. Bennett, Onset of a painful peripheral neuropathy in rat: a partial and differential differentiation and spontaneous discharge in Aβ and AO primary afferent neurons. J Neurophysiol,
Kang, Y., et al., Bidirectional Interactions between H-Channels and Na+-K+ Pumps in Mesencephalic Trigeminal Neurons. J. Neurosci., 2004. 24(14): p. 3694-3702.
Kaplan-Machlis, B. and B. S. Klostermeyer, The cyclooxygenase-2 inhibitors: safety and effectiveness. Ann Pharmacother, 1999. 33(9): p. 979-88.
Karbarz, M. J., et al., Biochemical and biological properties of 4-(3-phenyl-[1,2,4] thiadiazol-5-yl)-piperazine-1-carboxylic acid phenylamide, a mechanism-based inhibitor of fatty acid amide hydrolase. An
Katayama, K., et al., In vitro effect of N-methoxy-3-(3,5-ditert-butyl-4-hydroxybenzylidene)-2-pyrrolidon e (E-5110), a novel nonsteroidal antiinflammatory agent, on generation of some inflammatory mediator
Kaupp, U. B. and R. Seifert, Molecular diversity of pacemaker ion channels. Annu Rev Physiol, 2001. 63: p. 235-57.
Khandwala, H. and C. W. Loomis, Milacemide, a glycine pro-drug, inhibits strychnine-allodynia without affecting normal nociception in the rat. Pain, 1998. 77(1): p. 87-95.
Kim, S. H. and J. M. Chung, An experimental model for peripheral neuropathy produced by segmental spinal nerve ligation in the rat. Pain, 1992. 50(3): p. 355-63.
Kim, U. and D. A. McCormick, Functional and ionic properties of a slow after hyperpolarization in ferret perigeniculate neurons in vitro [In Process Citation]. J Neurophysiol, 1998. 80(3): p. 1222-1235.
Kitagawa, J., et al., Mechanisms involved in modulation of trigeminal primary afferent activity in rats with peripheral mononeuropathy. Eur J Neurosci, 2006. 24(7): p. 1976-86.
Klar, M., R. Surges, and T. J. Feuerstein, Ih channels as modulators of presynaptic terminal function: ZD7288 increases NMDA-evoked [3H]-noradrenaline release in rat neocortex slices. Naunyn Schmiedebergs A
Klusáková, I. and P. Dubový, Experimental models of peripheral neuropathic pain based on traumatic nerve injuries—an anatomical perspective. Ann Anat, 2009. 191(3): p. 248-59.
Knaus, A., et al., Direct inhibition of cardiac hyperpolarization-activated cyclic nucleotide-gated pacemaker channels by clonidine. Circulation, 2007. 115(7): p. 872-80.
Kole, M. H., S. Hallermann, and G. J. Stuart, Single Ih channels in pyramidal neuron dendrites: properties, distribution, and impact on action potential output. J Neurosci, 2006. 26(6): p. 1677-87.
Kouranova, E. V., et al., Hyperpolarization-activated cyclic nucleotide-gated channel mRNA and protein expression in large versus small diameter dorsal root ganglion neurons: Correlation with hyperpolarization-activated current gating. Neuroscience, 2008. 153(4): p. 1008-19.
Kramer, R. H. and J. W. Karpen, Spanning binding sites on allosteric proteins with polymer-linked ligand dimers. Nature, 1998. 395(6703): p. 710-3.
Krasowski, M. D., et al., 4D-QSAR analysis of a set of propofol analogues: mapping binding sites for an anesthetic phenol on the GABA(A) receptor. J Med Chem, 2002. 45(15): p. 3210-21.
Krasowski, M. D., et al., General anesthetic potencies of a series of propofol analogs correlate with potency for potentiation of g-aminobutyric acid (GABA) current at the GABA_{A} receptor but not with lipid solubility. J Pharmacol Exp Ther, 2001. 297(1): p. 338-51.
Krasowski, M. D., et al., General anesthetic potencies of a series of propofol analogs correlate with potency for potentiation of γ-aminobutyric acid (GABA) current at the GABAA receptor but not with lipid
Krasowski, M. D., et al., Methionine 286 in transmembrane domain 3 of the GABAA receptor beta subunit controls a binding cavity for propofol and other alkylphenol general anesthetics. Neuropharmacology, 200
Krasowski, M. D., et al., Propofol and other intravenous anesthetics have sites of action on the gammaaminobutyric acid type A receptor distinct from that for isoflurane. Mol Pharmacol, 1998. 53(3): p. 530-
Laird, J. M., et al., Deficits in visceral pain and referred hyperalgesia in Nav1.8 (SNS/PN3)-null mice. J Neurosci, 2002. 22(19): p. 8352-6.
Lazer, E. S., et al., Antiinflammatory 2,6-di-tert-butyl-4-(2-arylethenyl)phenols. J Med Chem, 1989. 32(1): p. 100-4.
Lazer, E. S., et al., Effect of structure on potency and selectivity in 2,6-disubstituted 4-(2-arylethenyl)phenol lipoxygenase inhibitors. J Med Chem, 1990. 33(7): p. 1892-8.
Lee, D. H., et al., Hyperpolarization-activated, cation-nonselective, cyclic nucleotide-modulated channel blockade alleviates mechanical allodynia and suppresses ectopic discharge in spinal nerve ligated rats. J Pain, 2005. 6(7): p. 417-24.
Lee, K. H. and D. A. McCormick, Abolition of spindle oscillations by serotonin and norepinephrine in the ferret lateral geniculate and perigeniculate nuclei in vitro. Neuron, 1996. 17(2): p. 309-321.
Lee, Y. T., et al., Novel pharmacological activity of loperamide and CP-339,818 on human HCN channels characterized with an automated electrophysiology assay. Eur J Pharmacol, 2008. 581(1-2): p. 97-104.
Lesch, C. A., et al., Effects of novel anti-inflammatory compounds on healing of acetic acid-induced gastric ulcer in rats. J Pharmacol Exp Ther, 1998. 287(1): p. 301-6.
Lingamaneni, R., et al., Anesthetic properties of 4-iodopropofol: implications for mechanisms of anesthesia. Anesthesiology, 2001. 94(6): p. 1050-7.
Lipscombe, D. and J. Q. Pan, Tripping the HCN breaker. Neuron, 2009. 62(6): p. 747-50.
Liu, C. N., et al., Spinal nerve injury enhances subthreshold membrane potential oscillations in DRG neurons: relation to neuropathic pain. J Neurophysiol, 2000. 84(1): p. 205-15.
Liu, C. N., et al., Subthreshold oscillations induced by spinal nerve injury in dissociated muscle and cutaneous afferents of mouse DRG. J Neurophysiol, 2002. 87(4): p. 2009-17.
Liu, C. N., et al., Tactile allodynia in the absence of C-fiber activation: altered firing properties of DRG neurons following spinal nerve injury. Pain, 2000. 85(3): p. 503-21.
Liu, D. T., G. R. Tibbs, and S. A. Siegelbaum, Subunit stoichiometry of cyclic nucleotide-gated channels and effects of subunit order on channel function. Neuron, 1996. 16(5): p. 983-90.
Loftsson, T. and M. E. Brewster, Pharmaceutical applications of cyclodextrins. 1. Drug solubilization and stabilization. J Pharm Sci, 1996. 85(10): p. 1017-25.
London, M. and M. Hausser, Dendritic computation. Annu Rev Neurosci, 2005. 28: p. 503-32.
Loomis, C. W., et al., Coadministration of intrathecal strychnine and bicuculline effects synergistic allodynia in the rat: an isobolographic analysis. J Pharmacol Exp Ther, 2001. 296(3): p. 756-61.
Lorenc, J. F., G. Lambeth, and W. Scheffer, in Kirk-Othmer Encyclopedia of Chemical Technology. 2003, John Wiley and Sons, Inc. p. 203-233.
Lorincz, A., et al., Polarized and compartment-dependent distribution of HCN1 in pyramidal cell dendrites. Nat Neurosci, 2002. 5(11): p. 1185-93.
Ludwig, A., et al., A family of hyperpolarization-activated mammalian cation channels. Nature, 1998. 393(6685): p. 587-91.
Ludwig, A., et al., Absence epilepsy and sinus dysrhythmia in mice lacking the pacemaker channel HCN2. Embo J, 2003. 22(2): p. 216-24.
Ludwig, A., et al., Two pacemaker channels from human heart with profoundly different activation kinetics. Embo J, 1999. 18(9): p. 2323-9.
Lujan, R., et al., Preferential localization of the hyperpolarization-activated cyclic nucleotide-gated cation channel subunit HCN1 in basket cell terminals of the rat cerebellum. Eur J Neurosci, 2005. 21(
Luo, L., et al., Role of peripheral hyperpolarization-activated cyclic nucleotide-modulated channel pacemaker channels in acute and chronic pain models in the rat. Neuroscience, 2007. 144(4): p. 1477-85.
Luthi, A. and D. A. McCormick, H-current: properties of a neuronal and network pacemaker. Neuron, 1998. 21(1): p. 9-12.
Luthi, A. and D. A. McCormick, Periodicity of thalamic synchronized oscillations: the role of Ca2+-mediated upregulation of Ih. Neuron, 1998. 20(3): p. 553-63.
Lyashchenko, A. K. and G. R. Tibbs, Ion binding in the open HCN pacemaker channel pore: fast mechanisms to shape “slow” channels. J Gen Physiol, 2008. 131(3): p. 227-43.
Lyashchenko, A. K., et al., Propofol inhibits HCN1 pacemaker channels by selective association with the closed states of the membrane embedded channel core. J Physiol, 2007. 583(Pt 1): p. 37-56.
Ma, W., W. Du, and J. C. Eisenach, Role for both spinal cord COX-1 and COX-2 in maintenance of mechanical hypersensitivity following peripheral nerve injury. Brain Res, 2002. 937(1-2): p. 94-9.
Maccaferri, G. and C. J. McBain, The hyperpolarization-activated current (Ih) and its contribution to pacemaker activity in rat CA1 hippocampal stratum oriens-alveus interneurones. J Physiol, 1996. 497 (Pt 1
MacKinnon, R., Potassium channels. FEBS Lett, 2003. 555(1): p. 62-5.
Magee, J. C. and D. Johnston, Plasticity of dendritic function. Curr Opin Neurobiol, 2005. 15(3): p. 334-42.
Magee, J. C., Dendritic Ih normalizes temporal summation in hippocampal CA1 neurons. Nat Neurosci, 1999. 2(9): p. 848.
Maher, M. P., et al., HCN channels as targets for drug discovery. Comb Chem High Throughput Screen, 2009. 12(1): p. 64-72.
Malan, T. P., Jr. and F. Porreca, Lipid mediators regulating pain sensitivity. Prostaglandins Other Lipid Mediat, 2005. 77(1-4): p. 123-30.
Malmberg, A. B. and A. I. Basbaum, Partial sciatic nerve injury in the mouse as a model of neuropathic pain: behavioral and neuroanatomical correlates. Pain, 1998. 76(1-2): p. 215-22.
Malosio, M. L., et al., Widespread expression of glycine receptor subunit mRNAs in the adult and developing rat brain. EMBO J, 1991. 10(9): p. 2401-9.
Mannikko, R., et al., Hysteresis in the voltage dependence of HCN channels: conversion between two modes affects pacemaker properties. J Gen Physiol, 2005. 125(3): p. 305-26.
Mannikko, R., F. Elinder, and H. P. Larsson, Voltage-sensing mechanism is conserved among ion channels gated by opposite voltages. Nature, 2002. 419(6909): p. 837-41.
Marik, P. E. and J. Varon, The management of status epilepticus. Chest, 2004. 126(2): p. 582-91.
Markin, V. S. and F. Sachs, Thermodynamics of mechanosensitivity. Phys Biol, 2004. 1(1-2): p. 110-24.
Markman, J. D. and R. H. Dworkin, Ion channel targets and treatment efficacy in neuropathic pain. J Pain, 2006. 7(1 Suppl 1): p. S38-47.
Mayer, M. L. and G. L. Westbrook, A voltage-clamp analysis of inward (anomalous) rectification in mouse spinal sensory ganglion neurones. J Physiol, 1983. 340: p. 19-45.
McCormick, D. A. and H. C. Pape, Properties of a hyperpolarization-activated cation current and its role in rhythmic oscillation in thalamic relay neurones. J Physiol (Lond), 1990. 431: p. 291-318.
McCormick, D. A. and T. Bal, Sleep and arousal: thalamocortical mechanisms. Annu Rev Neurosci, 1997. 20: p. 185-215.
McGowan, E., et al., A peripherally acting Na(v)1.7 sodium channel blocker reverses hyperalgesia and allodynia on rat models of inflammatory and neuropathic pain. Anesth Analg, 2009. 109(3): p. 951-8.
Migliore, M. and G. M. Shepherd, Emerging rules for the distributions of active dendritic conductances. Nat Rev Neurosci, 2002. 3(5): p. 362-70.
Migliore, M., L. Messineo, and M. Ferrante, Dendritic Ih selectively blocks temporal summation of unsynchronized distal inputs in CA1 pyramidal neurons. J Comput Neurosci, 2004. 16(1): p. 5-13.
Ming-Ju, H. and Y. Manyin, Theoretical AM1 studies of inclusion complexes of heptakis(2-Ohydroxypropyl)-beta-cyclodextrins with alkylated phenols. International Journal of Quantum Chemistry, 2004. 100(5):
Mistrik, P., et al., The murine HCN3 gene encodes a hyperpolarization-activated cation channel with slow kinetics and unique response to cyclic nucleotides. J Biol Chem, 2005. 280(29): p. 27056-61.
Momin, A., et al., Role of the hyperpolarization-activated current Iₕ in somatosensory neurons. J Physiol, 2008. 586(Pt 24): p. 5911-29.
Monteggia, L. M., et al., Cloning and localization of the hyperpolarization-activated cyclic nucleotidegated channel family in rat brain. Brain Res Mol Brain Res, 2000. 81(1-2): p. 129-39.
Moosmang, S., et al., Cellular expression and functional characterization of four hyperpolarization activated pacemaker channels in cardiac and neuronal tissues. Eur J Biochem, 2001. 268(6): p. 1646-52.
Moosmang, S., et al., Differential distribution of four hyperpolarization-activated cation channels in mouse brain. Biol Chem, 1999. 380(7-8): p. 975-80.
Morin, T. J. and W. R. Kobertz, Counting membrane-embedded KCNE beta-subunits in functioning K+ channel complexes. Proc Natl Acad Sci USA, 2008. 105(5): p. 1478-82.
Morin, T. J. and W. R. Kobertz, Tethering chemistry and K+ channels. J Biol Chem, 2008. 283(37): p. 25105-9.
Mosconi, T. and L. Kruger, Fixed-diameter polyethylene cuffs applied to the rat sciatic nerve induce a painful neuropathy: ultrastructural morphometric analysis of axonal alterations. Pain, 1996. 64(1): p.
Mourot, A. and R. H. Kramer, Staples, tape measures, and bungee cords: a variety of bifunctional reagents for understanding and controlling ion channels. ACS Chem Biol, 2007. 2(7): p. 451-3.
Much, B., Wahl-Schott, C., Zong, X., Schneider, A., Baumann, L., Moosmang, S., Ludwig, A., and Biel, M. Role of subunit heteromerization and N-linked glycosylation in the formation of functional HCN channels. 2003. J. Biol. Chem. 278:43781-43786.
Muller, F., et al., HCN channels are expressed differentially in retinal bipolar cells and concentrated at synaptic terminals. Eur J Neurosci, 2003. 17(10): p. 2084-96.
Mullican, M. D., et al., Design of 5-(3,5-di-tert-butyl-4-hydroxyphenyl)-1,3,4-thiadiazoles, -1,3,4-oxadiazoles, and -1,2,4-triazoles as orally-active, nonulcerogenic antiinflammatory agents. J Med Chem, 1993. 36(8): p. 1090-9.
Necula, M., Chirita, C. N., and Kuret, J. (2003) J Biol Chem 278, 46674-46680
Nishibe, Y. and M. Hirata, Induction of cytochrome P450 3A (CYP 3A) by E 5110, a non-steroidal antiinflammatory agent (NSAID), and typical CYP 3A inducers in primary cultures of dog hepatocytes. Biol Pharm
Nolan, M. F., et al., A behavioral role for dendritic integration: HCN1 channels constrain spatial memory and plasticity at inputs to distal dendrites of CA1 pyramidal neurons. Cell, 2004. 119(5): p. 719-32.
Nolan, M. F., et al., HCN1 channels control resting and active integrative properties of stellate cells from layer II of the entorhinal cortex. J Neurosci, 2007. 27(46): p. 12440-51.
Nolan, M. F., et al., The Hyperpolarization-Activated HCN1 Channel Is Important for Motor Learning and Neuronal Integration by Cerebellar Purkinje Cells. Cell, 2003. 115(5): p. 551-64.
Obreja, O., et al., A subpopulation of capsaicin-sensitive porcine dorsal root ganglion neurons is lacking hyperpolarization-activated cyclic nucleotide-gated channels. Eur J Pain, 2008. 12(6): p. 775-89.
O'Connor, A. B., Neuropathic pain: quality-of-life impact, costs and cost effectiveness of therapy. Pharmacoeconomics, 2009. 27(2): p. 95-112.
Oda, K., et al., Development of One-Pot Synthesis of New Antiarthritic Drug Candidate S-2474 with High E-Selectivity. Organic Process Research & Development, 2008. 12(3): p. 442-446.
Ogata, N. and Y. Ohishi, Molecular diversity of structure and function of the voltage-gated Na+ channels. Jpn J Pharmacol, 2002. 88(4): p. 365-77.
Orio, P., et al., Characteristics and physiological role of hyperpolarization activated currents in mouse cold thermoreceptors. J Physiol, 2009. 587(Pt 9): p. 1961-76.
Ossipov, M. H., et al., Spinal and supraspinal mechanisms of neuropathic pain. Ann N Y Acad Sci, 2000. 909: p. 12-24.
Ouyang, W., G. Wang, and H. C. Hemmings, Jr., Isoflurane and propofol inhibit voltage-gated sodium channels in isolated rat neurohypophysial nerve terminals. Mol Pharmacol, 2003. 64(2): p. 373-81.
Pal, M., et al., Vanilloid receptor antagonists: emerging class of novel anti-inflammatory agents for pain management. Curr Pharm Des, 2009. 15(9): p. 1008-26.
Pape, H. C., Queer current and pacemaker: the hyperpolarization-activated cation current in neurons. Annu Rev Physiol, 1996. 58: p. 299-327.
Patel, S., et al., The general anesthetic propofol increases brain N-arachidonylethanolamine (anandamide) content and inhibits fatty acid amide hydrolase. Br J Pharmacol, 2003. 139(5): p. 1005-13.
Pearce, R. J. and M. R. Duchen, Differential expression of membrane currents in dissociated mouse primary sensory neurons. Neuroscience, 1994. 63(4): p. 1041-56.
Perez, M., et al., Dimerization of sumatriptan as an efficient way to design a potent, centrally and orally active 5-HT1B agonist. Bioorg Med Chem Lett, 1998A. 8(6): p. 675-80.
Perez, M., et al., Dimers of 5HT1 ligands preferentially bind to 5HT1B/1D receptor subtypes. Bioorg Med Chem Lett, 1998B. 8(11): p. 1407-12.
Pistis, M., et al., The interaction of general anaesthetics with recombinant GABAA and glycine receptors expressed in Xenopus laevis oocytes: a comparative study. Br J Pharmacol, 1997. 122(8): p. 1707-19.
Pistis, M., et al., The interaction of general anaesthetics with recombinant GABAA and glycine receptors expressed in Xenopus laevis oocytes: a comparative study. Br J Pharmacol, 1997. 122(8): p. 1707-19.
Poolos, N. P., M. Migliore, and D. Johnston, Pharmacological upregulation of h-channels reduces the excitability of pyramidal neuron dendrites. Nat Neurosci, 2002. 5(8): p. 767-74.
Rajalu, M., et al., Plasticity of synaptic inhibition in mouse spinal cord lamina II neurons during early postnatal development and after inactivation of the glycine receptor a3 subunit gene. Eur J Neurosci, 2009. 30(12): p. 2284-92.
Rajewski, R. A. and V. J. Stella, Pharmaceutical applications of cyclodextrins. 2. In vivo drug delivery. J Pharm Sci, 1996. 85(11): p. 1142-69.
Ramsey, I. S., M. Delling, and D. E. Clapham, An introduction to TRP channels. Annu Rev Physiol, 2006. 68: p. 619-47.
Rehberg, B. and D. S. Duch, Suppression of central nervous system sodium channels by propofol. Anesthesiology, 1999. 91(2): p. 512-20.
Reyes, A., Influence of dendritic conductances on the input-output properties of neurons. Annu Rev Neurosci, 2001. 24: p. 653-75.
Robinson, R. B. and S. A. Siegelbaum, Hyperpolarization-Activated Cation Currents: From Molecules to Physiological Function. Annu Rev Physiol, 2003. 65: p. 453-480.
Robinson, R. B. and S. A. Siegelbaum, Hyperpolarization-Activated Cation Currents: From Molecules to Physiological Function. Annu Rev Physiol, 2002. 19: p. 19.
Romanelli, P. and V. Esposito, The functional anatomy of neuropathic pain. Neurosurg Clin N Am, 2004. 15(3): p. 257-68.
Rossetti, A. O., et al., Propofol treatment of refractory status epilepticus: a study of 31 episodes. Epilepsia, 2004. 45(7): p. 757-63.
Rothberg, B. S., Shin, K. S., Phale, P. S., and Yellen, G. (2002) J Gen Physiol 119, 83-91
Rothberg, B. S., et al., Voltage-controlled gating at the intracellular entrance to a hyperpolarization activated cation channel. J Gen Physiol, 2002. 119(1): p. 83-91.
Rothberg, B. S., K. S. Shin, and G. Yellen, Movements near the Gate of a Hyperpolarization-activated Cation Channel. J Gen Physiol, 2003.
Rowley, T. J., et al., The antinociceptive response to nicotinic agonists in a mouse model of postoperative pain. Anesth Analg, 2008. 107(3): p. 1052-7.
Roza, C., et al., The tetrodotoxin-resistant Na+ channel Nav1.8 is essential for the expression of spontaneous activity in damaged sensory axons of mice. J Physiol, 2003. 550(Pt 3): p. 921-6.
Rozario, A. O., et al., Voltage-dependent opening of HCN channels: Facilitation or inhibition by the phytoestrogen, genistein, is determined by the activation status of the cyclic nucleotide gating ring. Bi
Rudolph, U. and B. Antkowiak, Molecular and neuronal substrates for general anaesthetics. Nat Rev Neurosci, 2004. 5(9): p. 709-20.
Rusch, D., H. Zhong, and S. A. Forman, Gating allosterism at a single class of etomidate sites on alpha1beta2gamma2L GABA A receptors accounts for both direct activation and agonist modulation. J Biol Chem,
Samad, T. A., A. Sapirstein, and C. J. Woolf, Prostanoids and pain: unraveling mechanisms and revealing therapeutic targets. Trends Mol Med, 2002. 8(8): p. 390-6.
Santoro, B. and G. R. Tibbs, The HCN gene family: molecular basis of the hyperpolarization-activated pacemaker channels. Ann N Y Acad Sci, 1999. 868: p. 741-64.
Santoro, B., and Tibbs, G. R. (1999) Ann N Y Acad Sci 868, 741-764
Santoro, B., B. J. Wainger, and S. A. Siegelbaum, Regulation of HCN channel surface expression by a novel C-terminal protein-protein interaction. J Neurosci, 2004. 24(47): p. 10750-62.
Santoro, B., et al., Identification of a gene encoding a hyperpolarization-activated pacemaker channel of brain. Cell, 1998. 93(5): p. 717-29.
Santoro, B., et al., Interactive cloning with the SH3 domain of N-src identifies a new brain specific ion channel protein, with homology to eag and cyclic nucleotide-gated channels. Proc Natl Acad Sci U S, 1997. 94(26): p. 14815-20.
Santoro, B., et al., Molecular and functional heterogeneity of hyperpolarization-activated pacemaker channels in the mouse CNS. J Neurosci, 2000. 20(14): p. 5264-75.
Scholz, J. and C. J. Woolf, Can we conquer pain? Nat Neurosci, 2002. 5 Suppl: p. 1062-7.
Scholz, J. and C. J. Woolf, The neuropathic pain triad: neurons, immune cells and glia. Nat Neurosci, 2007. 10(11): p. 1361-8.
Schulze-Bahr, E., et al., Pacemaker channel dysfunction in a patient with sinus node disease. J Clin Invest, 2003. 111(10): p. 1537-45.
Scroggs, R. S., et al., Variation in I_{H}, I_{IR}, and I_{LEAK} between acutely isolated adult rat dorsal root ganglion neurons of different size. J Neurophysiol, 1994. 71(1): p. 271-9.
Seifert, R., et al., Molecular characterization of a slowly gating human hyperpolarization-activated channel predominantly expressed in thalamus, heart, and testis. Proc Natl Acad Sci USA, 1999. 96(16): p. 9391-6.
Seltzer, Z., R. Dubner, and Y. Shir, A novel behavioral model of neuropathic pain disorders produced in rats by partial sciatic nerve injury. Pain, 1990. 43(2): p. 205-18.
Servin, F., et al., Pharmacokinetics and protein binding of propofol in patients with cirrhosis. Anesthesiology, 1988. 69(6): p. 887-91.
Sherman, S. E. and C. W. Loomis, Morphine insensitive allodynia is produced by intrathecal strychnine in the lightly anesthetized rat. Pain, 1994. 56(1): p. 17-29.
Shin, J., et al., Bradykinin-12-lipoxygenase-VR1 signaling pathway for inflammatory hyperalgesia. Proc Natl Acad Sci USA, 2002. 99(15): p. 10150-5.
Shin, K. S., B. S. Rothberg, and G. Yellen, Blocker state dependence and trapping in hyperpolarization-activated cation channels: evidence for an intracellular activation gate. J Gen Physiol, 2001. 117(2): p. 91-101.
Shin, K. S., et al., Inactivation in HCN channels results from reclosure of the activation gate: desensitization to voltage. Neuron, 2004. 41(5): p. 737-44.
Shirota, H., et al., Inhibitory effects of E-5110 on interleukin-1 generation from human monocytes. Agents Actions, 1989. 27(3-4): p. 322-4.
Shirota, H., et al., Pharmacological properties of N-methoxy-3-(3,5-ditert-butyl-4-hydroxybenzylidene)-2-pyrrolidone (E-5110), a novel nonsteroidal antiinflammatory agent. Agents Actions, 1987. 21(3-4): p.
Siegelbaum, S. A., Presynaptic facilitation by hyperpolarization-activated pacemaker channels. Nat Neurosci, 2000. 3(2): p. 101-2.
Sietsema, W. K., et al., Absorption, bioavailability, and pharmacokinetics of tebufelone in the rat. J Pharm Sci, 1993. 82(6): p. 610-2.
Smith, C., et al., The interaction of fentanyl on the Cp₅₀ of propofol for loss of consciousness and skin incision. Anesthesiology, 1994. 81(4): p. 820-8.
Solomon, J. S. and J. M. Nerbonne, Hyperpolarization-activated currents in isolated superior colliculus projecting neurons from rat visual cortex. J Physiol (Lond), 1993. 462: p. 393-420.
Soltesz, I., et al., Two inward currents and the transformation of low-frequency oscillations of rat and cat thalamocortical cells. J Physiol (Lond), 1991. 441: p. 175-197.
Song, X. J., et al., Mechanical and thermal hyperalgesia and ectopic neuronal discharge after chronic compression of dorsal root ganglia. J Neurophysiol, 1999. 82(6): p. 3347-58.
Song, Y., et al., 2,6-di-tert-butylphenols: a new class of potent and selective PGHS-2 inhibitor. Inflamm Res, 1997. 46 Suppl 2: p. S141-2.
Song, Y., et al., Synthesis, structure-activity relationships, and in vivo evaluations of substituted di-tert-butylphenols as a novel class of potent, selective, and orally active cyclooxygenase-2 inhibitors. 1. Thiazolone and oxazolone series. J Med Chem, 1999. 42(7): p. 1151-60.
Southan, A. P. and B. Robertson, Patch-clamp recordings from cerebellar basket cell bodies and their presynaptic terminals reveal an asymmetric distribution of voltage-gated potassium channels. J Neurosci,
Southan, A. P., et al., Hyperpolarization-activated currents in presynaptic terminals of mouse cerebellar basket cells. J Physiol, 2000. 526 Pt 1: p. 91-7.
Steinmeyer, J., Pharmacological basis for the therapy of pain and inflammation with nonsteroidal antiinflammatory drugs. Arthritis Res, 2000. 2(5): p. 379-85.
Steriade, M., D. A. McCormick, and T. J. Sejnowski, Thalamocortical oscillations in the sleeping and aroused brain. Science, 1993. 262(5134): p. 679-85.
Stieber, J., et al., Functional expression of the human HCN3 channel. J Biol Chem, 2005. 280(41): p. 34635-43.
Stieber, J., et al., Molecular basis for the different activation kinetics of the pacemaker channels HCN2 and HCN4. J Biol Chem, 2003. 278(36): p. 33672-80.
Stieber, J., et al., The hyperpolarization-activated channel HCN4 is required for the generation of pacemaker action potentials in the embryonic heart. Proc Natl Acad Sci USA, 2003. 100(25): p. 15235-40.
Stieber, J., F. Hofmann, and A. Ludwig, Pacemaker channels and sinus node arrhythmia. Trends Cardiovasc Med, 2004. 14(1): p. 23-8.
Stocker, R., Molecular mechanisms underlying the antiatherosclerotic and antidiabetic effects of probucol, succinobucol, and other probucol analogues. Curr Opin Lipidol, 2009. 20(3): p. 227-35.
Sun, Q., et al., Ectopic discharges from injured nerve fibers are highly correlated with tactile allodynia only in early, but not late, stage in rats with spinal nerve ligation. Exp Neurol, 2005. 191(1): p.
Sun, Q., et al., Inhibition of hyperpolarization-activated current by ZD7288 suppresses ectopic discharges of injured dorsal root ganglion neurons in a rat model of neuropathic pain. Brain Res, 2005. 1032(1-2): p. 63-9.
Surges, R., T. M. Freiman, and T. J. Feuerstein, Input resistance is voltage dependent due to activation of Ih channels in rat CA1 pyramidal cells. J Neurosci Res, 2004. 76(4): p. 475-80.
Surmeier, D. J., J. N. Mercer, and C. S. Chan, Autonomous pacemakers in the basal ganglia: who needs excitatory synapses anyway? Curr Opin Neurobiol, 2005. 15(3): p. 312-8.
Swartz, K. J., Sensing voltage across lipid membranes. Nature, 2008. 456(7224): p. 891-7.
Teasell, R. W., et al., A systematic review of pharmacologic treatments of pain after spinal cord injury. Arch Phys Med Rehabil, 2010. 91(5): p. 816-31.
Thoby-Brisson, M., P. Telgkamp, and J. M. Ramirez, The role of the hyperpolarization-activated current in modulating rhythmic activity in the isolated respiratory network of mice. J Neurosci, 2000. 20(8): p.
Tibbs, G. R., E. H. Goulding, and S. A. Siegelbaum, Allosteric activation and tuning of ligand efficacy in cyclic-nucleotide-gated channels. Nature, 1997. 386(6625): p. 612-5.
Tibbs, G. R., et al., A state-independent interaction between ligand and a conserved arginine residue in cyclic nucleotide-gated channels reveals a functional polarity of the cyclic nucleotide binding site.
Tilley, S. L., T. M. Coffman, and B. H. Koller, Mixed messages: modulation of inflammation and immune responses by prostaglandins and thromboxanes. J Clin Invest, 2001. 108(1): p. 15-23.
Trapani, G., et al., Effect of 2-hydroxypropyl-β-cyclodextrin on the aqueous solubility of the anaesthetic agent propofol (2,6-diisopropylphenol) Int J Pharm, 1996. 139: p. 215-218.
Trapani, G., et al., Inclusion complexation of propofol with 2-hydroxypropyl-beta-cyclodextrin. Physicochemical, nuclear magnetic resonance spectroscopic studies, and anesthetic properties in rat. trapani@ippo.uniba.it. J Pharm Sci, 1998. 87(4): p. 514-8.
Trapani, G., et al., Propofol analogues. Synthesis, relationships between structure and affinity at GABAA receptor in rat brain, and differential electrophysiological profile at recombinant human GABAA rec
Trapani, G., et al., Propofol in anesthesia. Mechanism of action, structure-activity relationships, and drug delivery. Curr Med Chem, 2000. 7(2): p. 249-71.
Tsay, D., J. T. Dudman, and S. A. Siegelbaum, HCN1 channels constrain synaptically evoked Ca2+ spikes in distal dendrites of CA1 pyramidal neurons. Neuron, 2007. 56(6): p. 1076-89.
Tsuboi, Y., et al., Alteration of the second branch of the trigeminal nerve activity following inferior alveolar nerve transection in rats. Pain, 2004. 111(3): p. 323-34.
Tu, H., et al., Hyperpolarization-activated, cyclic nucleotide-gated cation channels: roles in the differential electrophysiological properties of rat primary afferent neurons. J Neurosci Res, 2004. 76(5): p. 713-22.
Udesky, J. O., et al., The role of nicotinic inhibition in ketamine-induced behavior. Anesth Analg, 2005. 101(2): p. 407-11, table of contents.
Ulens, C. and S. A. Siegelbaum, Regulation of Hyperpolarization-Activated HCN Channels by cAMP through a Gating Switch in Binding Domain Symmetry. Neuron, 2003. 40(5): p. 959-70.
Ulrich, D., Dendritic resonance in rat neocortical pyramidal cells. J Neurophysiol, 2002. 87(6): p. 2753-9.
Vanegas, H. and H. G. Schaible, Descending control of persistent pain: inhibitory or facilitatory? Brain Res Brain Res Rev, 2004. 46(3): p. 295-309.
Vanlersberghe, C. and F. Camu, Propofol. Handb Exp Pharmacol, 2008 (182): p. 227-52.
Vasilyev, D. V., et al., Direct inhibition of Ih by analgesic loperamide in rat DRG neurons. J Neurophysiol, 2007. 97(5): p. 3713-21.
Vemana, S., Pandey, S., and Larsson, H. P. (2003) J Gen Physiol; Mannikko, R., Elinder, F., and Larsson, H. P. 2002. Nature 419, 837-841.
Vemana, S., S. Pandey, and H. P. Larsson, S4 Movement in a Mammalian HCN Channel. J Gen Physiol, 2003.
Venkatachalam, K. and C. Montell, TRP channels. Annu Rev Biochem, 2007. 76: p. 387-417.
Viernstein, H., et al., Preparation and central action of propofol/hydroxypropyl-beta-cyclodextrin complexes in rabbits. Arzneimittelforschung, 1993. 43(8): p. 818-21.
Viscomi, C., et al., C terminus-mediated control of voltage and cAMP gating of hyperpolarization activated cyclic nucleotide-gated channels. J Biol Chem, 2001. 276(32): p. 29930-4.
Visser, E. and S. A. Schug, The role of ketamine in pain management. Biomed Pharmacother, 2006. 60(7): p. 341-8.
Vo, T., A. S. Rice, and R. H. Dworkin, Non-steroidal anti-inflammatory drugs for neuropathic pain: how do we explain continued widespread use? Pain, 2009. 143(3): p. 169-71.
Voets, T., et al., Sensing with TRP channels. Nat Chem Biol, 2005. 1(2): p. 85-92.
Voets, T., et al., The principle of temperature-dependent gating in cold- and heat-sensitive TRP channels. Nature, 2004. 430(7001): p. 748-54.
Vuyk, J., et al., Pharmacodynamics of propofol in female patients. Anesthesiology, 1992. 77(1): p. 3-9.
Wahl-Schott, C. and M. Biel, HCN channels: structure, cellular regulation and physiological function. Cell Mol Life Sci, 2009. 66(3): p. 470-94.
Wahl-Schott, C., et al., An arginine residue in the pore region is a key determinant of chloride dependence in cardiac pacemaker channels. J Biol Chem, 2005.
Wang, J., et al., Activity-dependent regulation of HCN pacemaker channels by cyclic AMP: signaling through dynamic allosteric coupling. Neuron, 2002. 36(3): p. 451-61.
Wang, J., S. Chen, and S. A. Siegelbaum, Regulation of Hyperpolarization-activated HCN Channel Gating and cAMP Modulation due to Interactions of COOH Terminus and Core Transmembrane Regions. J Gen Physiol,
Wang, X. Y., R. J. Motekaitis, and A. E. Martell, Metallotetraphenylporphyrin-catalyzed oxidation of 2,6-di-tert-butylphenol. Inorganic Chemistry, 1984. 23(3): p. 271-275.
Wasserman, M. A., et al., Chemistry and pharmacology of vascular protectants: a novel approach to the treatment of atherosclerosis and coronary artery disease. Am J Cardiol, 2003. 91(3A): p. 34A-40A.
Waxman, S. G. and B. C. Hains, Fire and phantoms after spinal cord injury: Na+ channels and central pain. Trends Neurosci, 2006. 29(4): p. 207-15.
Waxman, S. G., Neurobiology: a channel sets the gain on pain. Nature, 2006. 444(7121): p. 831-2.
Weisman, S. M., et al., Effects of tebufelone (NE-11740), a new anti-inflammatory drug, on arachidonic acid metabolism. Agents Actions, 1994. 41(3-4): p. 156-63.
Wells, J. E., K. C. Rowland, and E. K. Proctor, Hyperpolarization-activated channels in trigeminal ganglia innervating healthy and pulp-exposed teeth. Int Endod J, 2007. 40(9): p. 715-21.
Whitaker, G. M., et al., HCN2 and HCN4 Isoforms Self-assemble and Coassemble with Equal Preference to Form Functional Pacemaker Channels. J Biol Chem, 2007. 282(31): p. 22900-9.
Wickenden, A. D., M. P. Maher, and S. R. Chaplan, HCN pacemaker channels and pain: a drug discovery perspective. Curr Pharm Des, 2009. 15(18): p. 2149-68.
Williams, S. R., et al., On the nature of anomalous rectification in thalamocortical neurones of the cat ventrobasal thalamus in vitro. J Physiol (Lond), 1997. 505 (Pt 3): p. 727-747.
Willis, W. D., Jr., The somatosensory system, with emphasis on structures important for pain. Brain Res Rev, 2007. 55(2): p. 297-313
Wollmuth, L. P. and B. Hille, Ionic selectivity of Ih channels of rod photoreceptors in tiger salamanders. J Gen Physiol, 1992. 100(5): p. 749-765.
Wood, J. N., et al., Voltage-gated sodium channels and pain pathways. J Neurobiol, 2004. 61(1): p. 55-71.
Wu, G., et al., Early onset of spontaneous activity in uninjured C-fiber nociceptors after injury to neighboring nerve fibers. J Neurosci, 2001. 21(8): p. RC140.
Xie, W., et al., Neuropathic pain: early spontaneous afferent activity is the trigger. Pain, 2005. 116(3): p. 243-56.
Yagami, T., et al., Effects of S-2474, a novel nonsteroidal anti-inflammatory drug, on amyloid beta protein-induced neuronal cell death. Br J Pharmacol, 2001. 134(3): p. 673-81.
Yagami, T., et al., S-2474, a novel nonsteroidal anti-inflammatory drug, rescues cortical neurons from human group IIA secretory phospholipase A(2)-induced apoptosis. Neuropharmacology, 2005. 49(2): p. 174
Yamamoto, A., A unique antilipidemic drug—probucol. J Atheroscler Thromb, 2008. 15(6): p. 304-5.
Yamashita, S. and Y. Matsuzawa, Where are we with probucol: a new life for an old drug? Atherosclerosis, 2009. 207(1): p. 16-23.
Yao, H., et al., Upregulation of the hyperpolarization-activated cation current after chronic compression of the dorsal root ganglion. J Neurosci, 2003. 23(6): p. 2069-74.
Ye, B. and J. M. Nerbonne, Proteolytic Processing of HCN2 and Co-assembly with HCN4 in the Generation of Cardiac Pacemaker Channels. J Biol Chem, 2009. 284(38): p. 25553-9.
Yellen, G. The voltage-gated potassium channels and their relatives, (2002) Nature 419, 35-42.
Yellen, G., The moving parts of voltage-gated ion channels. Q Rev Biophys, 1998. 31(3): p. 239-95.
Ying, S. W., et al., Dendritic HCN2 channels constrain glutamate-driven excitability in reticular thalamic neurons. J Neurosci, 2007. 27(32): p. 8719-32.
Ying, S. W., et al., Propofol block of Iₕ contributes to the suppression of neuronal excitability and rhythmic burst firing in thalamocortical neurons. Eur J Neurosci, 2006. 23(2): p. 465-80.
Yu, H. Y. and J. K. Liau, Quantitation of propofol in plasma by capillary gas chromatography. J Chromatogr, 1993. 615(1): p. 77-81.
Zhang, Q., et al., Associated changes in HCN2 and HCN4 transcripts and 1(f) pacemaker Current in myocytes. Biochim Biophys Acta, 2009.
Zhao, F. Y., et al., GW406381, a novel COX-2 inhibitor, attenuates spontaneous ectopic discharge in sural nerves of rats following chronic constriction injury. Pain, 2007. 128(1-2): p. 78-87.
Zhao, X., et al., In vitro characterization of HCN channel kinetics and frequency dependence in myocytes predicts biological pacemaker functionality. J Physiol, 2009. 587(Pt 7): p. 1513-25.
Zhou, L. and S. A. Siegelbaum, Gating of HCN channels by cyclic nucleotides: residue contacts that underlie ligand binding, selectivity, and efficacy. Structure, 2007. 15(6): p. 655-70.

1-31. (canceled)

32. A method of managing or treating chronic pain comprising administering to a patient in need thereof an effective amount of a propofol derivative structurally modified to have limited general anesthetic properties, wherein the propofol derivative is a compound selected from the group consisting of: 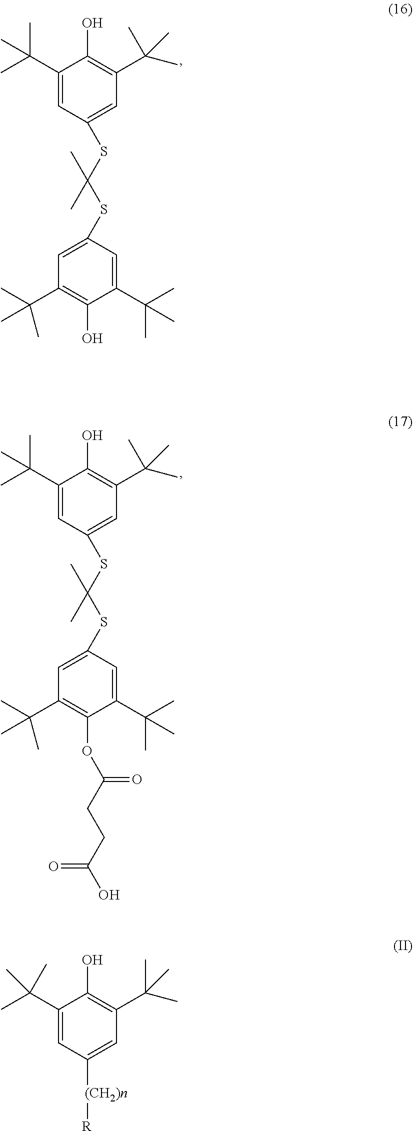
wherein
n=2-36; and
R is a positively charged group,
pharmaceutically acceptable salts of (16), (17), and (II), and combinations thereof.

33. The method according to claim 32, wherein R is selected from the group consisting of NH₂, N(CH₃)₃, a guanidine group, an aromatic amino group, and a quaternary ammonium group.

34. The method according to claim 32, wherein the propofol derivative is administered as part of a pharmaceutically acceptable composition.

35. The method according to claim 34, wherein the pharmaceutically acceptable composition is administered in a unit dosage form.

36. The method according to claim 35, wherein the propofol derivative is present in the unit dosage form at a total concentration of about 1 μM to about 20 μM.

37. The method according to claim 32, wherein the chronic pain is a neuropathic pain characterized by one or more symptoms selected from the group consisting of persistent negative sensory perception, hyperalgesia, allodynia, burning sensation, and unusual nociceptive descriptors.

38. A method of modulating HCN channel gating comprising providing to an HCN channel an effective amount of a propofol derivative having limited general anesthetic properties, wherein the propofol derivative is a compound selected from the group consisting of: 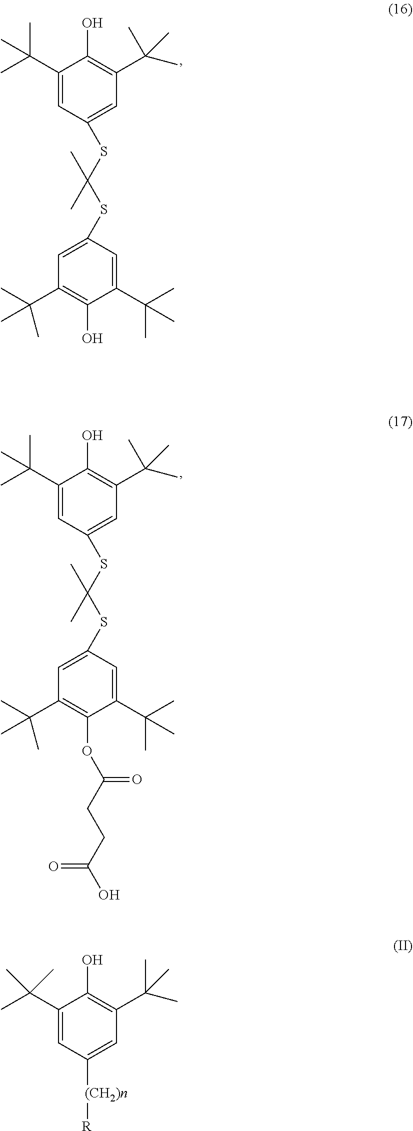
wherein
n=2-36; and
R is a positively charged group,
pharmaceutically acceptable salts of (16), (17), and (II), and combinations thereof.

39. The method according to claim 38, wherein the HCN channel is an HCN1 channel.

40. The method according to claim 38, wherein R is selected from the group consisting of NH₂, N(CH₃)₃, a guanidine group, an aromatic amino group, and a quaternary ammonium group.

41. The method according to claim 38, wherein the propofol derivative is administered as part of a pharmaceutically acceptable composition.

42. The method according to claim 41, wherein the pharmaceutically acceptable composition is administered in a unit dosage form.

43. The method according to claim 42, wherein the propofol derivative is present in the unit dosage form at a total concentration of about 1 μM to about 20 μM.

44. A method of inhibiting an HCN1 channel without enhancing a gamma-aminobutyric acid-A (GABA-A) receptor comprising providing to an HCN channel an effective amount of a propofol derivative having limited general anesthetic properties, wherein the propofol derivative is a compound selected from the group consisting of: 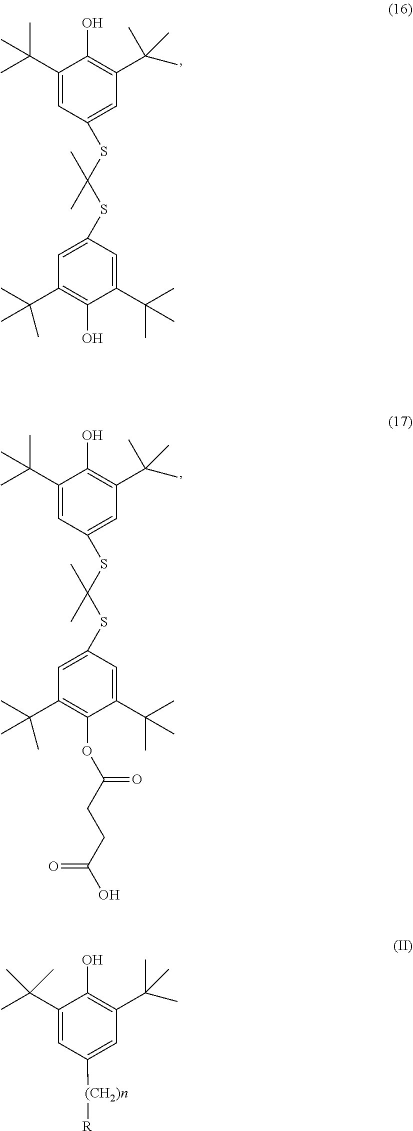
wherein
n=2-36; and
R is a positively charged group,
pharmaceutically acceptable salts of (16), (17), and (II), and combinations thereof.

45. The method according to claim 44, wherein R is selected from the group consisting of NH₂, N(CH₃)₃, a guanidine group, an aromatic amino group, and a quaternary ammonium group.

46. The method according to claim 44, wherein the propofol derivative is administered as part of a pharmaceutically acceptable composition.

47. The method according to claim 46, wherein the pharmaceutically acceptable composition is administered in a unit dosage form.

48. The method according to claim 47, wherein the propofol derivative is present in the unit dosage form at a total concentration of about 1 μM to about 20 μM.
